(19) United States
(12) Patent Application Publication
(10) Pub. No.: US 2023/0059009 A1
(43) Pub. Date: Feb. 23, 2023

(54) INHIBITORS OF LIN28 AND METHODS OF USE THEREOF

(71) Applicant: The Regents of the University of California, Oakland, CA (US)

(72) Inventors: Martina Roos, Los Angeles, CA (US); Michael E. Jung, Los Angeles, CA (US); Hyo Jin Gim, Los Angeles, CA (US)

(21) Appl. No.: 17/786,764

(22) PCT Filed: Dec. 14, 2020
(86) PCT No.: PCT/US20/64896

§ 371 Date: Jun. 17, 2022

Related U.S. Application Data
(60) Provisional application No. 62/949,873, filed on Dec. 18, 2019.

(51) Int. Cl. C07D 487/04; A61P 35/02; C07D 471/04

(52) U.S. Cl. CPC C07D 487/04; A61P 35/02; C07D 471/04

(57) ABSTRACT

The present disclosure relates to compounds of formula (I) and compositions comprising the same. The disclosure further relates to methods of treating cancers.

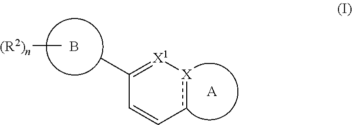

### RELATED APPLICATION

This application claims a right of priority to and the benefit of U.S. Provisional Patent Application No. 62/949,873, filed on Dec. 18, 2019, which is hereby incorporated by reference in its entirety.

### GOVERNMENT SUPPORT

This invention was made with government support under Grant Number TR001881 awarded by the National Institutes of Health. The government has certain rights in the invention.

### BACKGROUND

Acute myeloid leukemia (AML), is a hematologic malignancy characterized by clonal proliferation of myeloid blasts resulting in a fatal outcome for the majority of adults afflicted (1). Even under very aggressive multi-agent chemotherapy regimens, newer targeted therapies and myeloablative allogeneic hematopoietic cell transplants, the majority of patients succumb to AML within 5 years. Therapy resistant leukemic stem cells (LSCs) are thought to be the root cause of high relapse rates and treatment failure (2-4). Thus, the development of novel therapeutic strategies capable of eradicating LSCs represents a major area of unmet medical need.

Small molecules have proved to be successful therapeutics in clinical applications targeting proteins implicated in pathogenesis. However, currently FDA-approved drugs, directed to G-protein-coupled receptors, kinases, peptidases, nuclear receptors, proteases, ion channels, enzymes and others, modulate fewer than 700 human-genome derived proteins (63). This implies that less than 0.5% of the proteome and <0.05% of the genome has been explored as a target for therapeutic approaches. In addition, the majority of small molecule drugs in clinical use exploit structured binding pockets on protein surfaces. Allosteric and/or conformational changes on remote catalytic or drug-binding regions result in drug resistance and ultimately ineffectiveness of medications (64). Therefore, the development of novel drugs capable of targeting yet unexplored signaling pathways and overcoming resistance mutations represent a major area of unmet medical need.

### SUMMARY OF THE INVENTION

The present disclosure provides compounds of formula (I):

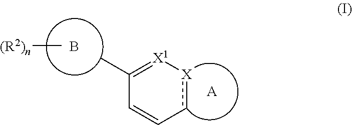

or a pharmaceutically acceptable salt thereof, wherein:

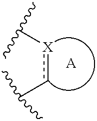

is selected from

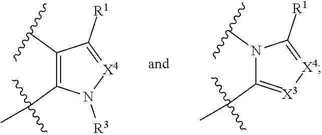

Ring B is selected from phenyl and a 5- to 6-membered heteroaryl ring having 1-3 heteroatoms independently selected from nitrogen, oxygen, and sulfur;
X is selected from N and C;
each of X¹, X³, and X⁴ is independently selected from N and C—R^{x};
R¹ is hydrogen or an optionally substituted group selected from C₁₋₆ aliphatic, phenyl, and a 5- to 6-membered heteroaryl ring having 1-3 heteroatoms independently selected from nitrogen, oxygen, and sulfur;
each R² is independently selected from hydrogen, halogen, NO₂, N(R)₂, OR, N(R)C(O)R, CO₂R, C(O)N(R)₂, and optionally substituted C₁₋₆ aliphatic;
R³ is selected from hydrogen and an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, and a 5- to 6-membered heteroaryl ring having 1-3 heteroatoms independently selected from nitrogen, oxygen, and sulfur;
each R^{x} is independently selected from hydrogen, halogen, and optionally substituted C₁₋₆ aliphatic;
each R is independently selected from hydrogen and an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, and a 5- to 6-membered heteroaryl ring having 1-3 heteroatoms independently selected from nitrogen, oxygen, and sulfur; and
n is 0-3.

In certain aspects, the present disclosure provides compounds of formula (II) and pharmaceutically acceptable salts thereof:

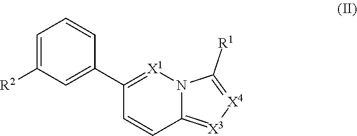

wherein:
R¹ is C₁₋₆ alkyl or C₃₋₆ cycloalkyl;
R² is H, amino, nitro, or acylamino; and
X¹, X³, and X⁴ are each independently N or CH.

In certain aspects, the present disclosure relates to pharmaceutical compositions comprising a compound disclosed herein and a pharmaceutically acceptable excipient.

In certain aspects, the present disclosure relates to methods of inhibiting Lin28 in cells, comprising contacting a cell comprising Lin28 with a compound or composition disclosed herein.

In certain aspects, the present disclosure relates to methods of treating cancer, comprising administering to a subject in need thereof a compound or composition disclosed herein.

### BRIEF DESCRIPTION OF THE DRAWINGS

FIG. 1 shows the impact of the Lin28/let-7 pathway on multiple pathways driving LSC proliferation. Upregulation of let-7 miRNAs by inhibition of LIN28 exerts a tumor suppressive function though down-regulation of genes which promote LSC proliferation (MYC, RAS, IL-6, CCND) and survival (BCL-2) and indirect inhibition of the NF-κB pathway.

FIGS. 2A-2D show Lin28b expression is increased in LSCs.

FIG. 2A shows Log 2 expression of Lin28b in healthy HSCs compared to cells from various AML karyotypes, including inv(16), t(8;21), t(11q23)/ILL, complex and normal karyotypes.

FIG. 2B shows Lin28b expression in non-DOX induced LT-HSCs compared to DOX induced MLL-AF9 WBM- and LT-HSC AML cells (→ WBM-AML, LSCs) and at relapse after Ara-C treatment (→ rLSCs). Gene expression is normalized to Lin28b of non-DOX induced LT-HSCs. n=5.

FIG. 2C shows relative let-7a and -b miRNA expression in LSCs pre- and post-relapse normalized to non-induced, LT-HSCs. n=3.

FIG. 2D shows CFC numbers of 1000 WBM- or 100 LT-HSC derived AML cells treated with control, 100 nM Ara-C or 30 μM 1632 (n=6) or transduction with shLin28b or shScramble (n=3) 7 d post plating *** P<0.001, ** P<0.01, * P<0.05.

FIGS. 3A-3B show LN1632 inhibits LIN28B protein expression.

FIG. 3A shows Westernblot of Kasumi-1 and THP-1 cells treated with 1632 (120-160 μM).

FIG. 3B shows TF1-alpha cells treated with 10 nM Bortezomib (BZ), 120 μM 1632 or a combination thereof.

FIGS. 4A-4C show Targeted Lin28/let-7 inhibition abrogates AML growth.

FIG. 4A shows treatment with 100 mg/kg 1632 significantly slowed tumor growth (picture). n=5. SubQ implanted THP-1 cells (high LIN28B).

FIG. 4B shows treatment with 100 mg/kg 1632 had minimal effect on tumor growth, n=7. SubQ implanted MOLM-13 AML cells (no LIN28B).

FIG. 4C shows treatment with 1632 at 100 mg/kg every other day prolongs survival (left) and reduces tumor burden (BLI, picture at right, taken at d+26, as indicated by black arrow) of a systemic Kasumi-1 AML cell model. n=5. Error bar represent SD.

FIGS. 5A-5C show Targeted Lin28 inhibition downregulates LSC driver genes.

FIG. 5A heatmap shows expression of direct (green) and indirect (black) let-7 target genes and pathways downregulated (MYC, NF-κB, JAK/STAT) in Kasumi-1 cells after treatment with 100 μM 1632 or control.

FIG. 5B: miRNA and let-7 target genes fold changes assessed in three primary AML patient samples after treatment with 1632 at 80-120 μM or control, n=3. Error bars are SEM. *** P<0.001, ** P<0.01, * P<0.05.

FIG. 5C show gene set enrichment analysis (GSEA), plots evaluating changes in LSC gene signatures (GAL, top) and relapse prognosis in pediatric AML (Yagi, bottom) in Kasumi-1 cells after treatment with 100 μM 1632 or control. NES, normalized enrichment score; FDR q-value, false discovery rate.

FIGS. 6A-6C show pharmacologic LIN28 inhibition selectively abrogates LSC repopulation capability in vivo.

FIG. 6A shows CFC numbers of AML pt #13 and healthy donor CD34+ cells after treatment with and without 80-120 μM 1632, Error bars represent SD, * P<0.05.

FIG. 6B shows Engraftment of primary human AML cells of pt #13 in NSGS 12 weeks post ex vivo treatment with control or 1632 at 120 μM. *** P<0.001.

FIG. 6C shows the representative flow cytometry gating scheme of engraftment of human AML cells treated with 120 μM 1632 or control for 72 h 12 weeks post transplantation into NSGS.

FIG. 7 shows that exemplary compounds of the present disclosure inhibit LIN28B binding to pre-let-7a. Compounds are screened in biological triplicates at doses of 20, 5, 1.25 μM. Signal response was corrected for compound self-fluorescence. Dotted line indicates highest FRET-signal achieve by hit compound LN1632. All compounds higher than the dotted line have increased inhibitory activity on LIN28B/pre-let-7a-2 binding.

FIGS. 8A-8C show binding of LN1632 to ZKD motif of LIN28 and upregulation of let-7.

FIG. 8A is the predicted binding mode of LN1632 to ZKDs of LIN28B. Close-contact interactions indicated in red lines, LN1632 purple.

FIG. 8B is the percent (%) inhibition of LIN28B-binding activity to pre-let-7a as measured by increased FRET signal intensity. Values were normalized to negative control treatment, n=3.

FIG. 8C shows the relative levels of functional let-7 miRNAs in HepG2 cells after treatment with LN1632 and analogs at concentrations from 3-10 μM. Values were normalized to total plasmid expression and control treatment, n=6. ** P<0.01, error bars are SEM, *** P<0.01.

FIGS. 9A-9C depict downregulation of cancer driver gene signatures by LN1632.

FIG. 9A is a heatmap showing genes of HALLMARK_MYC_TARGETS_V1 in Kasumi-1 cells after treatment with 40 μM LN1632 or control.

FIG. 9B shows gene set enrichment analysis (GSEA), plots evaluating changes in LSC gene signatures (GAL, top) and relapse prognosis in pediatric AML (Yagi, bottom) in Kasumi-1 cells after treatment with 40 μM LN1632 or control. NES, normalized enrichment score; FDR q-value, false discovery rate.

FIG. 9C shows biological functional analysis of RNAseq data of Kasumi-1 cells after treatment with 40 μM LN1632 or control. Ingenuity pathway analysis (IPA) predicts upstream inhibition of MYC and IL-6 pathway from differentially expressed genes in Kasumi-1 cells after treatment with 40 μM LN1632 or control (p-value: <0.05). The figure represents genes that are associated with a particular biological function that are altered in the uploaded dataset. Genes that are up-regulated are displayed within red nodes and those down-regulated are displayed within green nodes. The intensity of the color in a node indicates the degree of up-(red) or down-(green) regulation. The shapes of the nodes reflect the functional class of each gene product: transcriptional regulator (horizontal ellipse), transmembrane receptor (vertical ellipse), enzyme (vertical rhombus), cytokine/growth factor (square), kinase (inverted triangle) and complex/group/other (circle). An orange line indicates predicted upregulation, whereas a blue line indicates predicted downregulation. A yellow line indicates expression being contradictory to the prediction. Gray line indicates that direction of change is not predicted. Solid or broken edges indicate direct or indirect relationship, respectively

FIGS. 10A-10B show that LN1632 is well tolerated in healthy C57BL/6 female mice. FIG. 10A is a series of graphs showing CBCs (white blood cell counts (WBC), neutrophils (NEU), Lymphocytes (LYMPH), platelets (PLT) and hemoglobin (Hb)) levels in female C57Bl/6 mice treated daily TP with LN1632 at 100 mg/kg for +12 d, followed by every-other-day injections for +9 d, n=5.

FIG. 10B shows no significant changes in weight gain with treatment of LN1632 or vehicle at +21 d. n=5. Statistics: Two-ailed Student's t-test, error bars are SEM. * P<0.05.

FIGS. 11A-11C depict inhibition of cancer proliferation in vivo by LN1632.

FIG. 11A shows daily treatment with 100 mg/kg LN1632 significantly slowed tumor growth, n=5. Subcutaneously implanted THP-1 cells.

FIG. 11B shows systemic Kasumi-1 AML Xenograft. Treatment with 100 mg/kg LN1632 every other day prolongs survival and reduces tumor burden (picture, taken at d+26) of a systemic Kasumi-1 AML Xenograft. n=5.

FIG. 11C Subcutaneously implanted THP-1 cells showed inhibited proliferation when treated with LN1632 but to a lesser degree when treated with Ara-C. n=3. Statistics: Two-tailed Student's t-test, *** P<0.001, error bars are SEM.

FIGS. 12A-12C show target engagement of LN1632.

FIG. 12A shows mass spectrometry cellular thermal shift assay (MS-CETSA): incubation with LN1632 induces Tm shift of endogenous PRPF31 in Kasumi-1 cell lysate.

FIG. 12B shows mass spectrometry following immunoprecipitation (IP-MS) of biotinylated LN1632 and competitive elution with non-labelled LN1632 captures PRPF31, n=3.

FIG. 12C shows candidate targets of LN1632 identified by MS-CETSA and IP-MS, sorted by abundance, and overlapping faction.

FIG. 13 shows a correlation of PRPF31 overexpression with poor prognosis. Kaplan-Meier overall survival curves for patients with different cancer cohort's analysis. The p-values were calculated using the log-rank test. Vertical hash marks indicate censored data. The survival curve comparing the patient with high (red) and low (black) expression of PRPF31.

FIGS. 14A-14D show dependence of TNBC proliferation on PRPF31.

FIG. 14A shows cell numbers of TNBC cells treated with pCMV-PRPF31 expressing plasmid (red), control vector (pCMV-empty, black), shPRPF31 (green) or combinations of pCMV-PRPF31+100 μM LN1632, pCMV-GFP+100 μM LN1632 or shPRPF31+100 μM LN1632. n=3.

FIG. 14B shows % cell viability of MDA-MB-231 cells assessed by cell titer glow after treatment with LN1632, JGJ023, JGJ034 or Palbociclib in increasing doses for 4 days post plating, n=2.

FIG. 14C shows cell numbers of MDA-MB-231 cells incubated with control (DMSO), 16 μM JGJ023 or 16 μM Palbociclib at day +6, +9 and +12 post treatment, n=2.

FIG. 14D shows direct comparison of cell numbers of MDA-MB-231 cells at d+6 post treatment with 16 μM JGJ023 or 16 μM Palbociclib, n=2. Statistics: dose response curve for IC50 calculation plotted as four-parametric linear regression, two-tailed Student's t-test for individual comparison, error bars are SD, * P<0.05, ** P<0.01.

FIGS. 15A-15C depict inducement of apoptosis in castration resistant prostate cancer by LN1632 and novel analogs thereof.

FIG. 15A shows % cell viability of CRPC LNCaP cells expressing wild-type androgen receptor after treatment with LN1632, JGJ007, JGJ023 or standard of care Enzalutamide in increasing doses for 4 days.

FIG. 15B shows % cell viability of metastatic CRPC 22Rv1 cells expressing mutant androgen receptor (ARV7) after treatment with LN1632, JGJ007, JGJ023 or standard of care Enzalutamide in increasing doses for 4 days.

FIG. 15C shows cell numbers of 22Rv1 cells incubated with control (DMSO), 2 μM JGJ023 or 42 μM Enzalutamide at day +5, +7 and +9 post treatment. Small figure: JGJ023 induces apoptosis and reduces cell numbers of mCRPC compared to Enzalutamide. All experiments n=3. Statistics: dose response curve for IC₅₀ calculation plotted as four-parametric linear regression, two-tailed Student's t-test for individual dose comparison error bars are SD, * P<0.05, ** P<0.01.

FIGS. 16A-16C shows inducement of apoptosis and inhibition of proliferation of colorectal cancer by LN1632 and novel analogs thereof.

FIG. 16A shows % cell viability of epithelial CRC cells SW948, expressing low MYC (87), after treatment with JGJ034 or standard of care Cetuximab (EGFR monoclonal antibody) in increasing doses for 4 days.

FIG. 16B shows % cell viability of adenocarcinoma CRC cells SW480, with low MYC-amplification (88) after treatment with JGJ034 or standard of care Cetuximab in increasing doses for 4 days.

FIG. 16C shows % cell viability of Cetuximab-resistant, metastatic adenocarcinoma CRC cells SW620, with high MYC-amplification³⁹ after treatment with JGJ034 or standard of care Cetuximab in increasing doses for 5 days. All experiments n=3. Statistics: dose response curve for IC50 calculation plotted as four-parametric linear regression, error bars are SD.

### DETAILED DESCRIPTION OF THE INVENTION

Compounds

The present disclosure provides a compound of formula (I):

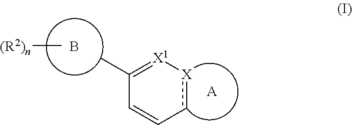

or a pharmaceutically acceptable salt thereof, wherein:

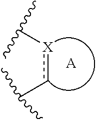

is selected from

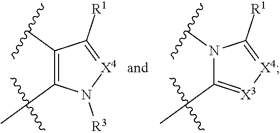

Ring B is selected from phenyl and a 5- to 6-membered heteroaryl ring having 1-3 heteroatoms independently selected from nitrogen, oxygen, and sulfur;
X is selected from N and C;
each of X¹, X³ and X⁴ is independently selected from N and C—R^{x};
R¹ is hydrogen or an optionally substituted group selected from C₁₋₆ aliphatic, phenyl, and a 5- to 6-membered heteroaryl ring having 1-3 heteroatoms independently selected from nitrogen, oxygen, and sulfur;
each R² is independently selected from halogen, NO₂, N(R)₂, OR, N(R)C(O)R, CO₂R, C(O)N(R)₂, and optionally substituted C₁₋₆ aliphatic;
R³ is selected from hydrogen and an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, and a 5- to 6-membered heteroaryl ring having 1-3 heteroatoms independently selected from nitrogen, oxygen, and sulfur;
each R^{x} is independently selected from hydrogen, halogen, and optionally substituted C₁₋₆ aliphatic;
each R is independently selected from hydrogen and an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, and a 5- to 6-membered heteroaryl ring having 1-3 heteroatoms independently selected from nitrogen, oxygen, and sulfur; and
n is 0-3.

In some embodiments of formula (I),

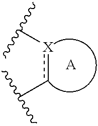

is

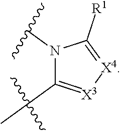

Accordingly, in some embodiments, the present disclosure provides a compound of formula (I-a):

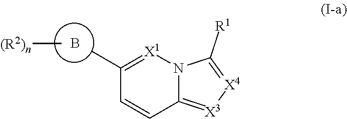

or a pharmaceutically acceptable salt thereof, wherein each of Ring B, X¹, X³, X⁴, R¹, R², and n is as defined above and described herein.

In some embodiments of formula (I),

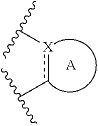

is

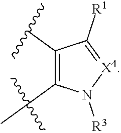

Accordingly, in some embodiments, the present disclosure provides a compound of formula (I-b):

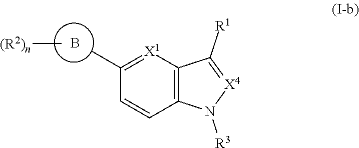

or a pharmaceutically acceptable salt thereof, wherein each of Ring B, X¹, X⁴, R¹, R², R, and n is as defined above and described herein.

As defined generally above, X¹ is selected from N and C—R^{x}. In some embodiments of any of formulae (I), (I-a), and (I-b), X¹ is N. Accordingly, in some embodiments, the present disclosure provides a compound of formulae (I-a-i) or (I-b-i):

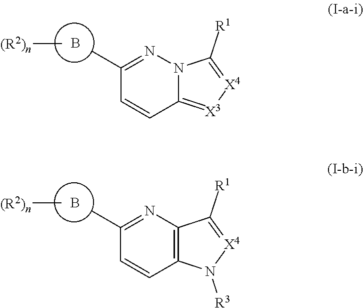

or a pharmaceutically acceptable salt thereof, wherein each of Ring B, X³, X⁴, R¹, R², R, and n is as defined above and described herein.

In some embodiments of any of formulae (I), (I-a), and (I-b), X¹ is C—R^{x}. Accordingly, in some embodiments, the present disclosure provides a compound of formulae (I-a-ii) or (I-b-ii):

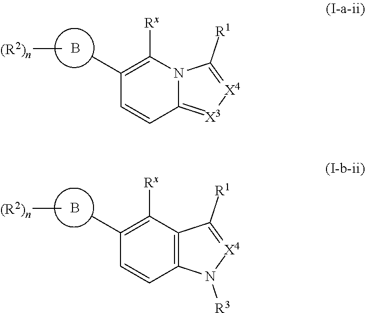

or a pharmaceutically acceptable salt thereof, wherein each of Ring B, X³, X⁴, R¹, R², R, R^{x}, and n is as defined above and described herein.

As defined generally above for formula (I), Ring B is selected from phenyl and a 5- to 6-membered heteroaryl ring having 1-3 heteroatoms independently selected from nitrogen, oxygen, and sulfur. In some embodiments of formulae (I), (I-a), (I-a-i), (I-a-ii), (I-b), (I-b-i), and (I-b-ii), Ring B is phenyl. Accordingly, in some embodiments, the present disclosure provides a compound of formulae (I-a-iii), (I-a-iv), (I-a-v), (I-b-iii), (I-b-iv), and (I-b-v):

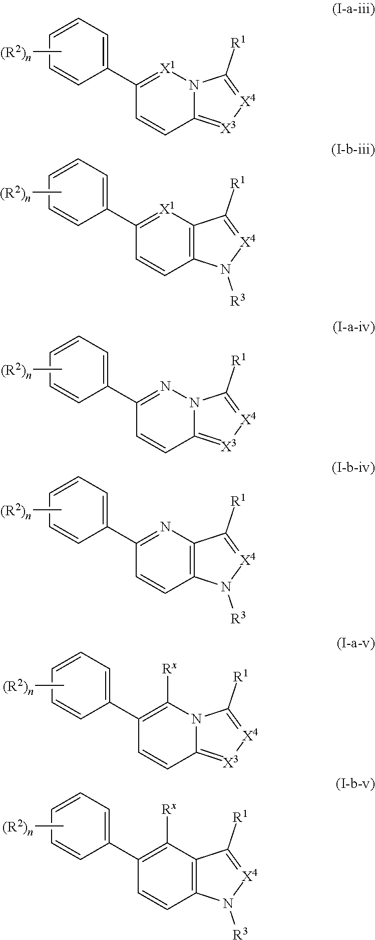

or a pharmaceutically acceptable salt thereof, wherein each of X¹, X³, X⁴, R¹, R², R, R^{x}, and n is as defined above and described herein.

In some embodiments of formulae (I), (I-a), (I-a-i), (I-a-ii), (I-b), (I-b-i), and (I-b-ii), Ring B is

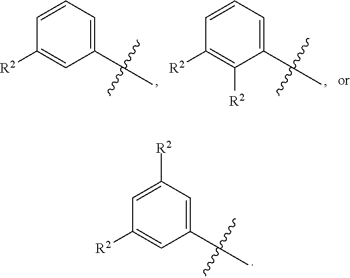

In some embodiments of formulae (I), (I-a), (I-a-i), (I-a-ii), (I-b), (I-b-i), and (I-b-ii), Ring B is a 5- to 6-membered heteroaryl ring having 1-3 heteroatoms independently selected from nitrogen, oxygen, and sulfur. In other embodiments of formulae (I), (I-a), I-a-i), I-a-ii), (I-b), (I-b-i), and (I-b-ii), Ring B is a 5-membered heteroaryl ring having 1-3 heteroatoms independently selected from nitrogen, oxygen, and sulfur. In other embodiments of formulae (I), (I-a), (I-a-i), (I-a-ii), (I-b), (I-b-i), and (I-b-ii), Ring B is a 6-membered heteroaryl ring having 1-2 nitrogen atoms, e.g., pyridyl.

As defined generally above for formula (I), X³ is selected from N and C—R^{x}. In some embodiments of any of formulae (I), (I-a), (I-a-i), (I-a-ii), (I-a-iii), (I-a-iv), and (I-a-v), X³ is N. In other embodiments of any of formulae (I), (I-a), (I-a-i), (I-a-ii), (I-a-iii), (I-a-iv), and (I-a-v), X³ is C—R^{x}.

As defined generally above for formula (I), X⁴ is selected from N and C—R^{x}. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), X⁴ is N. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), X⁴ is C—R^{x}.

As defined generally above for formula (I), R¹ is hydrogen or an optionally substituted group selected from C₁₋₆ aliphatic, phenyl, and a 5- to 6-membered heteroaryl ring having 1-3 heteroatoms independently selected from nitrogen, oxygen, and sulfur. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R¹ is hydrogen. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R¹ is an optionally substituted group selected from C₁₋₆ aliphatic, phenyl, and a 5- to 6-membered heteroaryl ring having 1-3 heteroatoms independently selected from nitrogen, oxygen, and sulfur. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R¹ is optionally substituted C₁₋₆ aliphatic. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R¹ is optionally substituted C₁₋₃ aliphatic, e.g., CH₃, CH₂CH₃, CH₂CH₂CH₃, or CH(CH₃)₂. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R¹ is optionally substituted phenyl. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R¹ is an optionally substituted 5- to 6-membered heteroaryl ring having 1-3 heteroatoms independently selected from nitrogen, oxygen, and sulfur. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R¹ is an optionally substituted 5-membered heteroaryl ring having 1-3 heteroatoms independently selected from nitrogen, oxygen, and sulfur. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R¹ is an optionally substituted 6-membered heteroaryl ring having 1-2 nitrogen atoms, e.g., pyridyl or pyrimidinyl.

As defined generally above for formula (I), R² is selected from halogen, NO₂, N(R)₂, OR, N(R)C(O)R, CO₂R, C(O)N(R)₂, and optionally substituted C₁₋₆ aliphatic. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is halogen. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is NO₂. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is OR, e.g., OMe.

In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is N(R)₂. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is NHR, e.g., NH₂.

In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is N(R)C(O)R, e.g., N(CH₃)C(O)CH₃. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is NHC(O)R, e.g., NHC(O)CH₃.

In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is CO₂R, e.g., CO₂H.

In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is C(O)N(R)₂. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is C(O)N(H)R, e.g., C(O)NHCH₃.

In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is optionally substituted C₁₋₆ aliphatic. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), at least one R² is optionally substituted C₁₋₃ aliphatic.

As defined generally above for formula (I), each R^{x} is independently selected from hydrogen, halogen, and optionally substituted C₁₋₆ aliphatic. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R^{x} is hydrogen. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R^{x} is independently selected from halogen and optionally substituted C₁₋₆ aliphatic. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R^{x} is halogen, e.g., fluoro or chloro.

In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R^{x} is optionally substituted C₁₋₆ aliphatic. In other embodiments, R^{x} is optionally substituted C₁₋₃ aliphatic, e.g., CH₃, CH₂CH₃, CH₂CH₂CH₃, or CH(CH₃)₂.

As defined generally above for formula (I), each R is independently selected from hydrogen or an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, a 5- to 6-membered heteroaryl ring having 1-3 heteroatoms independently selected from nitrogen, oxygen, and sulfur. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R is hydrogen. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R is independently selected from an optionally substituted group selected from C₁₋₆ aliphatic, 3- to 7-membered monocyclic carbocyclic ring, a 3- to 7-membered monocyclic heterocyclic ring having 1-3 heteroatoms independently selected from nitrogen, oxygen, and sulfur, phenyl, a 5- to 6-membered heteroaryl ring having 1-3 heteroatoms independently selected from nitrogen, oxygen, and sulfur. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R is optionally substituted C₁₋₆ aliphatic. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), R is optionally substituted C₁₋₃ aliphatic. In some such embodiments, R is CH₃ or CH₂CH₃.

As defined generally above for formula (I), n is 0-3. In some embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), n is 1-2 In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), n is 0. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), n is 1. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), n is 2. In other embodiments of any of formulae (I), (I-a), (I-a-i), (I-a-ii), (I-a-iii), (I-a-iv), (I-a-v), (I-b), (I-b-i), (I-b-ii), (I-b-iii), (I-b-iv), and (I-b-v), n is 3.

In some embodiments of any of the disclosed compounds, R¹ is C₁₋₆ aliphatic, e.g., methyl or propyl. In other embodiments, R¹ is phenyl. In other embodiments, R¹ is a 5- to 6-membered heteroaryl ring having 1-3 heteroatoms independently selected from oxygen, nitrogen, and sulfur. In other embodiments, wherein R¹ is a 5-membered heteroaryl ring having 1-3 heteroatoms independently selected from nitrogen, oxygen, and sulfur. In other embodiments, wherein R¹ is a 6-membered heteroaryl ring having 1-3 nitrogen atoms. In other embodiments, R¹ is a 6-membered heteroaryl ring having 1-2 nitrogen atoms, e.g.,

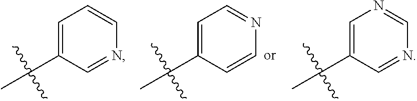 [SMILES: *c1cccnc1 ; *c1ccncc1 ; *c1cncnc1]

In some embodiments of any of the disclosed compounds, R^{x} is hydrogen. In other embodiments, R^{x} is halogen or optionally substituted C₁₋₆ aliphatic. In other embodiments, R^{x} is optionally substituted C₁₋₆ aliphatic. In other embodiments, R^{x} is unsubstituted C₁₋₆ aliphatic, e.g., methyl.

In some embodiments of any of the disclosed compounds, R² is selected from halogen, NO₂, N(R)₂, OR, N(R)C(O)R, CO₂R, C(O)N(R)₂, and optionally substituted C₁₋₆ aliphatic. In other embodiments, R² is halogen, e.g., fluoro. In other embodiments, R² is NO₂. In other embodiments, R² is OR, e.g., OCH₃. In other embodiments, wherein R² is N(R)₂, e.g., NH₂. In other embodiments, R² is N(R)C(O)R, e.g., NHC(O)CH₃ or N(CH₃)C(O)CH₃. In other embodiments, R² is CO₂R, e.g., CO₂H. In other embodiments, R² is C(O)N(R)₂, e.g., C(O)NHCH₃. In other embodiments, R² is optionally substituted C₁₋₆ aliphatic, e.g., CF₃.

In some embodiments of any of the disclosed compounds, R is hydrogen. In other embodiments, R is an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, a 5- to 6-membered heteroaryl ring having 1-3 heteroatoms independently selected from nitrogen, oxygen, and sulfur. In other embodiments, R is optionally substituted C₁₋₆ aliphatic. In other embodiments, R is unsubstituted C₁₋₆ aliphatic, e.g., methyl.

In some embodiments of any of the disclosed compounds, R³ is hydrogen. In other embodiments, R³ is an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, a 5- to 6-membered heteroaryl ring having 1-3 heteroatoms independently selected from nitrogen, oxygen, and sulfur. In other embodiments, R³ is optionally substituted C₁₋₆ aliphatic. In other embodiments, R³ is unsubstituted C₁₋₆ aliphatic, e.g., methyl.

In some embodiments of any of the disclosed compounds, R³ is hydrogen. In other embodiments, R³ is an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, a 5- to 6-membered heteroaryl ring having 1-3 heteroatoms independently selected from nitrogen, oxygen, and sulfur. In other embodiments, R³ is optionally substituted C₁₋₆ aliphatic. In some embodiments, R³ is unsubstituted C₁₋₆ aliphatic, e.g., methyl.

In some embodiments of any of the disclosed compounds, n is 0. In other embodiments, n is 1. In other embodiments, n is 2. In some embodiments, the present disclosure provides a compound selected from:

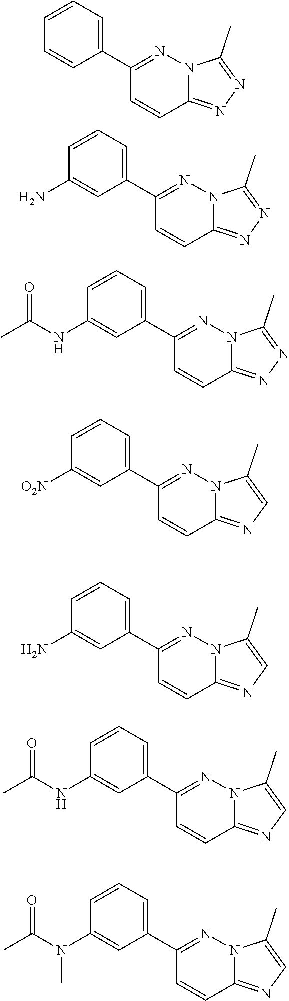

or a pharmaceutically acceptable salt thereof.

In some embodiments, the present disclosure provides a compound selected from:

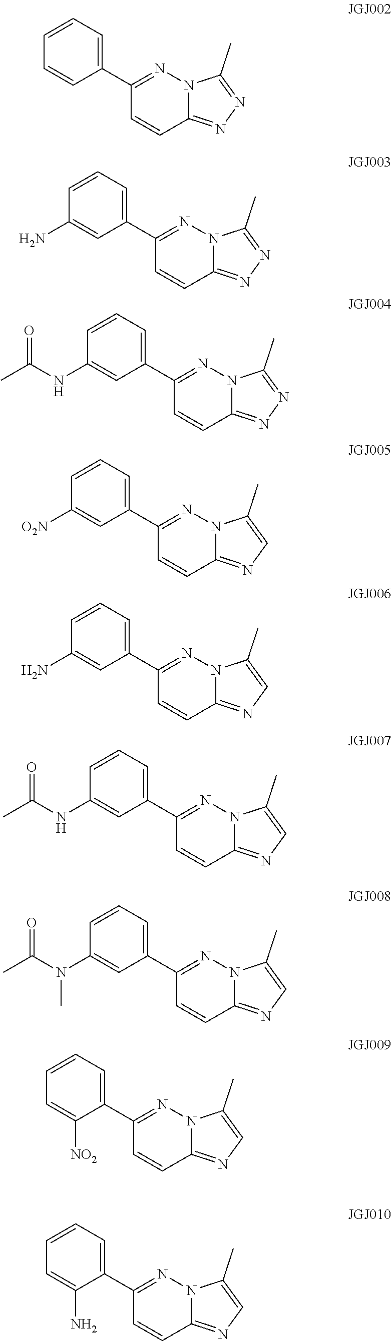

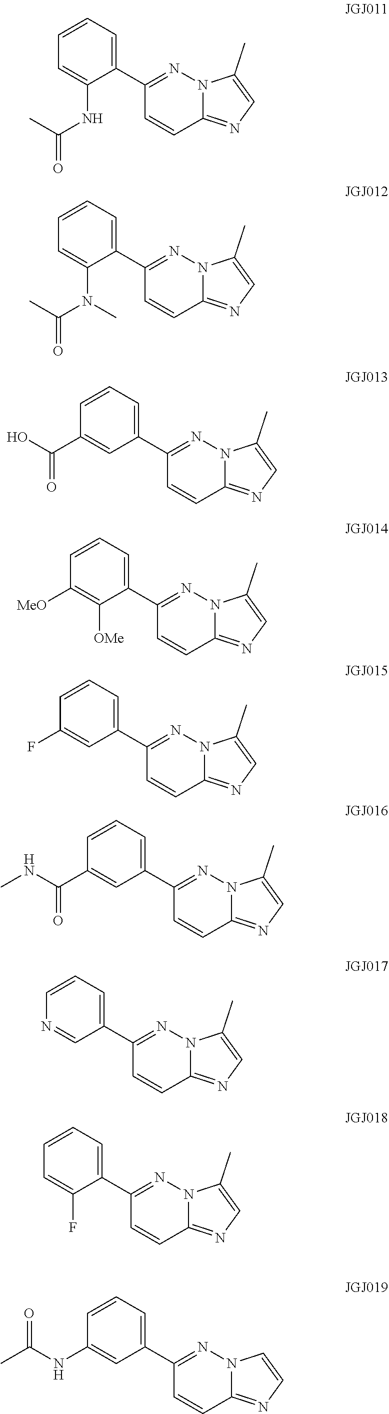

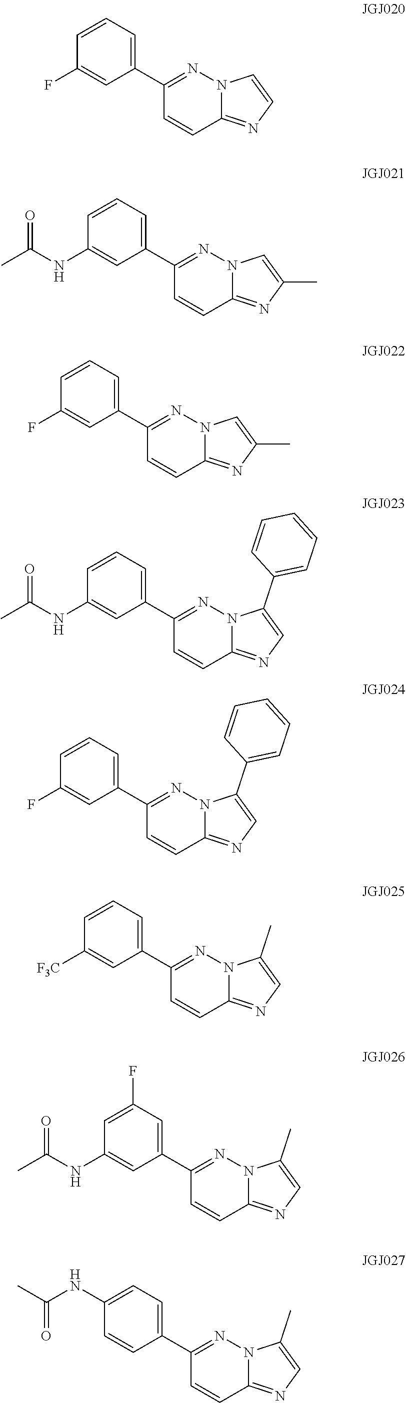

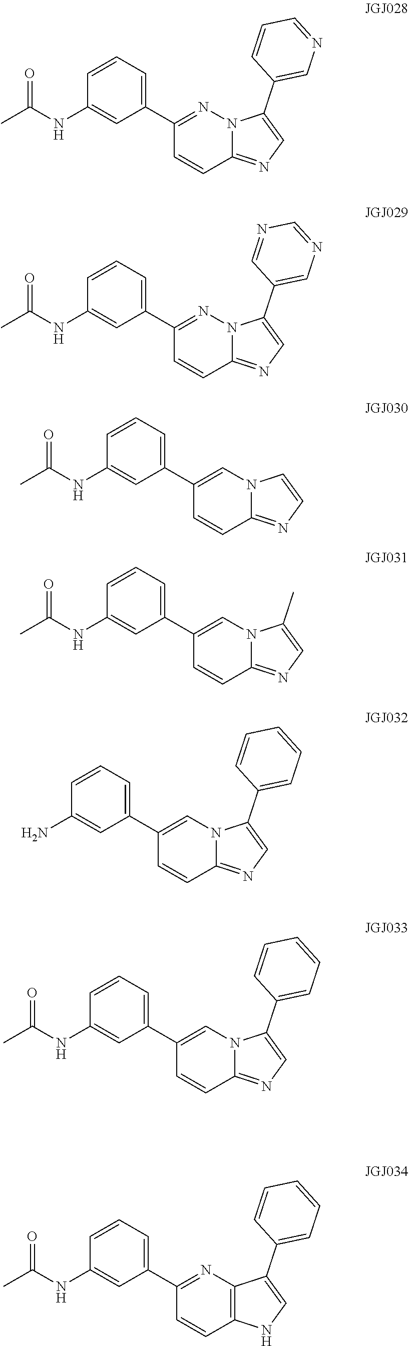

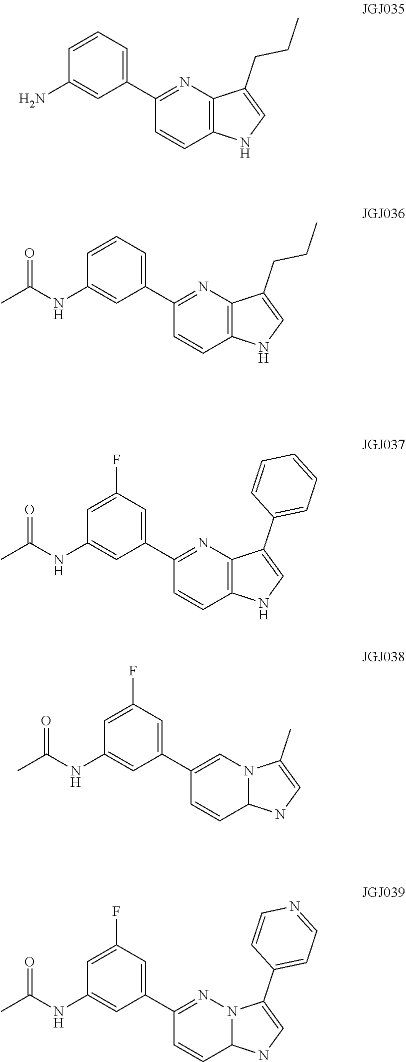

or a pharmaceutically acceptable salt thereof.

In some embodiments, the present disclosure provides a compound of formula (II):

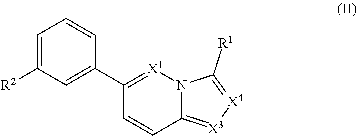

or a pharmaceutically acceptable salt thereof, wherein:
R¹ is C₁₋₆ alkyl or C₃₋₆ cycloalkyl;
R² is H, amino, nitro, or acylamino; and
X¹, X³, and X⁴ are each independently N or CH.

In some embodiments, at least one of X¹, X³, and X⁴ is N. In other embodiments, at least two of X¹, X³, and X⁴ are N. In other embodiments, each of X¹, X³, and X⁴ is N. In other embodiments, X¹ and X³ are each N, and X⁴ is CH.

In some embodiments, R¹ is unsubstituted C₁₋₆ alkyl, e.g., methyl. In other embodiments, R¹ is methyl optionally substituted with halogen. In other embodiments, R¹ is C₂₋₆ alkyl or C₃₋₆ cycloalkyl.

In some embodiments, R² is H, amino, nitro, or —N(R⁵)C(O)R⁶; R⁵ is H or C₁₋₅ alkyl; and

R⁶ is C₁₋₆ alkyl. In other embodiments, R² is —N(R⁵)C(O)R⁶, R⁵ is H, and R⁶ is C₁₋₆ alkyl. In other embodiments, R² is —N(R⁵)C(O)R⁶, R⁵ is H, and R⁶ is CH₃. In other embodiments, R² is H, amino, or nitro. In other embodiments, R² is NO₂ or —N(R⁵)C(O)R⁶.

In some embodiments, wherein the compound is:

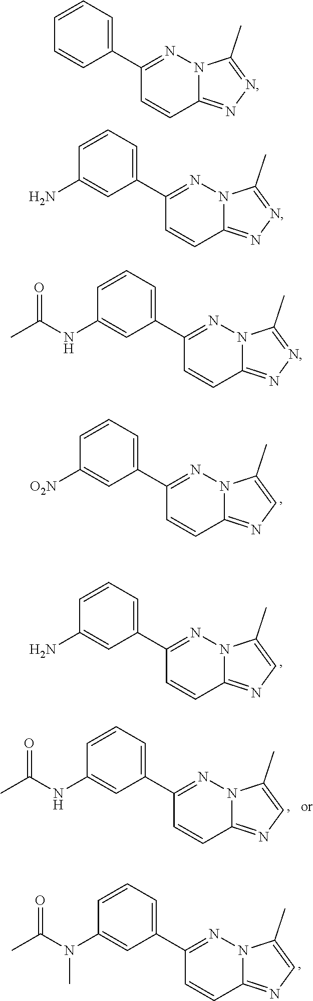

or a pharmaceutically acceptable salt thereof.

In some embodiments, wherein the compound is JGJ002, JGJ003, JGJ004, JGJ005, JGJ007, or JGJ008, or a pharmaceutically acceptable salt thereof.

In some embodiments, wherein the compound is JGJ007 or JGJ088, or a pharmaceutically acceptable salt thereof.

In some embodiments of formula (II), X¹ is N. Accordingly, in some embodiments, the present disclosure provides a compound of formula (II-a):

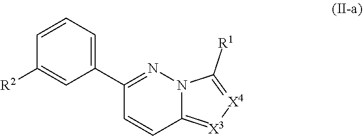

or a pharmaceutically acceptable salt thereof, wherein each of R¹, R², X³, and X⁴ is as defined above sand described herein.

In some embodiments of formula (II), X³ is N. Accordingly, in some embodiments, the present disclosure provides a compound of formula (I-b):

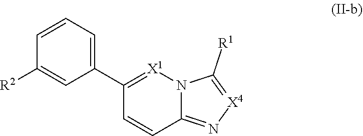

or a pharmaceutically acceptable salt thereof, wherein each of R¹, R², X¹, and X⁴ is as defined above and described herein.

In some embodiments of formula (II-a), X³ is N. Accordingly, in some embodiments, the present disclosure provides a compound of formula (I-a-i):

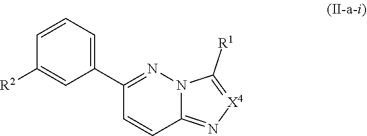

or a pharmaceutically acceptable salt thereof, wherein each of R¹, R², and X⁴ is as defined above and described herein.

As defined generally above for formula (II), R¹ is C₁₋₆ alkyl or C₃₋₆ cycloalkyl. In other embodiments of any of formulae (II), (II-a), (II-b), and (II-a-i), R¹ is C₁₋₆ alkyl. In other embodiments of any of formulae (II), (II-a), (II-b), and (II-a-i), R¹ is C₁₋₃ alkyl, e.g., R¹ is CH₃, CH₂CH₃, CH₂CH₂CH₃, or CH(CH₃)₂.

In other embodiments of any of formulae (II), (II-a), (II-b), and (II-a-i), R¹ is C₃₋₆ cycloalkyl, e.g., cyclopropyl, cyclobutyl, cyclopentyl, or cyclohexyl. In other embodiments of any of formulae (II), (II-a), (II-b), and (II-a-i), R¹ is cyclopropyl or cyclobutyl. In other embodiments of any of formulae (II), (II-a), (II-b), and (II-a-i), R¹ is cyclopentyl or cyclohexyl.

As defined generally above for formula (II), R² is H, amino, nitro, or acylamino. In some embodiments of any of formulae (II), (II-a), (II-b), and (II-a-i), R² is H. In other embodiments of any of formulae (II), (II-a), (II-b), and (II-a-i), R² is amino, nitro, or acylamino. In other embodiments of any of formulae (II), (II-a), (II-b), and (II-a-i), R² is amino. In other embodiments of any of formulae (II), (II-a), (II-b), and (II-a-i), R² is nitro. In other embodiments of any of formulae (II), (II-a), (II-b), and (II-a-i), R² is acylamino (e.g.,

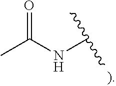 [SMILES: *NC(C)=O]

In some embodiment, the amino is N(R)₂.

In some embodiment, the acylamino is N(R)C(O)R.

In some embodiments of any of the disclosed compounds, the compound is not

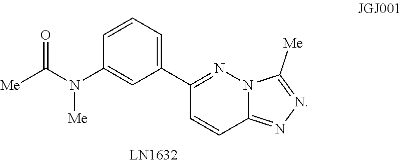 [SMILES: CC(=O)N(C)c1cccc(-c2ccc3nnc(C)n3n2)c1]

Pharmaceutical Compositions and Uses Thereof

In some embodiments, the present disclosure provides the recognition that methods for targeting ribonucleic acid (RNA)—RNA-binding protein (RBP) interactions constitute an emerging alternative approach to significantly expand on the druggable proteome and genome and to overcome intrinsic and acquired resistance.

In certain aspects, the present disclosure further provides the recognition that RBPs play a crucial role in cellular physiology by regulating RNA processing, translation, and turnover. In neoplasms, dysregulated expression of RBPs support expression of alternatively spliced, modified and stabilized RNA transcripts that are relevant to cancer self-renewal, proliferation and its adaption to stress. In some embodiments, the present disclosure provides compounds that modulate distinct RBP-protein interactions and therefore represent a novel therapeutic approach to treat cancer and other diseases with dysfunctional RNA regulation.

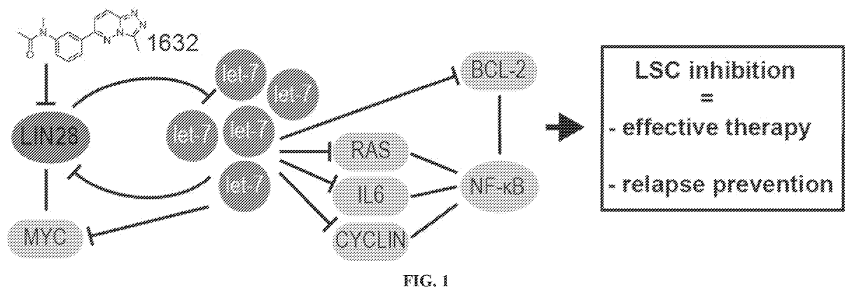

MicroRNAs (miRNAs) are 19-22 nucleotide (nt) short non-coding RNAs that hybridize to complementary mRNA targets and lead either to their decay, cleavage or transcriptional inhibition (5-7). Aberrant miRNA expression has been shown to play an active role in malignant transformation, including leukemia (8-10). Specifically, in AML let-7b and let-7c miRNAs were found to be significantly downregulated in core binding factor (CBF) leukemias with inv(16), t(8;21) and MLL/t(11q23) (11) (12). Systematic evaluation of the prognostic value of miRNA expression many human cancers, including several AML subtypes, found that decreased expression of let-7 miRNAs is frequently associated with poor prognosis (10, 13, 14). The let-7 tumor suppressor miRNA family comprises 12 members that are differentially transcribed from eight chromosomal loci and repress several cancer stem cell oncogenes including KRAS, MYC, IL6 and HMGA1/2 as well as cell cycle regulators such as CCND1/2 and E2F (FIG. 1) (15, 16). In 2008, a rush of papers described LIN28A and its homologue LIN28B (hereafter referred to LIN28) as key regulators of let-7 biogenesis (17-21) through direct binding to either pre-let-7 and/or pri-let-7, thereby impairing their processing into mature, functional miRNAs. In fact, LIN28 is upregulated in more 15% of human cancers (22) and cancer stem cells (CSCs) (23-27).

Structural studies revealed that Lin28's C-terminal zinc-knuckle domains (ZKDs) binds a highly conserved GGAG motif within the 3′ terminal loop of the pri-/pre-let-7 (28-30). This binding results in recruitment of TUTases to polyuridinylate pre/pri-let-7 thereby blocking maturation of let-7 miRNAs (19, 31). Thus, decreased let-7 miRNA leads to overexpression of their directly regulated oncogenic target genes.

The RNA-binding proteins LIN28A and LIN28B are overexpressed in many cancers, with high LIN28 protein correlating with reduced patient survival (54). LIN28A/B (hereinafter referred as Lin28) impairs the processing of functional mature let-7 microRNAs (miRNAs) by binding with its C-terminal zinc-knuckle domains (ZKDs) to a highly conserved GGAG motif within the 3′ terminal loop of pri-/pre-let-7 (17-21, 28-30). As a result, in some embodiments, decreased let-7 miRNAs lead to overexpression of their direct oncogenic target genes, such as MYC, KRAS, and CCND1. Besides its ability to suppress let-7 miRNA biogenesis, Lin28 has been shown to bind mRNA transcripts of the insulin like growth factor 2 proteins (Igf2), thereby affecting their abundance and/or translation (69, 70).

In a wide variety of cancers, increasing evidence shows that LIN28 overexpression (32-34) and let-7 loss (35-37) are associated with resistance CSCs to radiation treatment and chemotherapy, ultimately leading to reduced overall survival. Specifically, in AML, dysregulated LIN28/let-7 has been shown to promote leukemogenesis via an LSC-like transcriptional program and is associated with poor clinical outcome (38). In bone marrow aspirates from patients with refractory AML, let-7a has been found to confer Ara-C chemotherapy resistance through BCL-XL, a BCL-2 family member (39). Importantly, several studies have emphasized that overexpression of BCL-2 and BCL-XL in AML and in LSCs specifically, are associated with chemotherapy resistance and poor overall/disease-free survival (40-43). In addition, let-7 miRNAs target IL6 and RAS, two well-known genetic drivers of the NF-κB pathway, another important regulator of LSC homeostasis (44) (FIG. 1).

Emerging evidence shows that NF-κB and BCL-2 are activated in LSCs, but not hematopoietic stem cells (HSCs), as a central component of the pro-inflammatory cellular stress response (45, 46). Hence, therapeutic inhibition of LIN28 and consequently upregulation of let-7 may selectively kill LSCs. Given the fundamental role of Lin28/let-7 in leukemic and other CSCs and its relevance to therapy resistance, it is conceivable that targeted inhibition of LIN28 may be a novel approach for precision AML therapy. Of note, studies in conditional Lin28a and Lin28b knockout mice revealed that fetal but not neonatal or adult Lin28 deficiency resulted in growth defects (47), suggesting that Lin28 has heterochronic effects. Moreover, in mice, expression of the Lin28b was found to decrease in hematopoietic stem cells (48, 49) and coincided with accumulation of mature let-7 in common myeloid progenitors during hematopoietic maturation (50). Thus, therapeutic inhibition of LIN28 and the resulting upregulation of let-7 miRNAs could selectively kill LSCs but will likely have high tolerability to healthy tissues.

To date, five high-throughput screens (HTS) have been reported with the objective of identifying of pharmacologically active compounds that disrupt LIN28 from binding to pre-let-7 miRNA. We screened 16,000 drug-like organic compounds using a FRET-HTS and identified the first hit compound 501632 (51) (hereafter referred as LN1632) to bind LIN28B and selectively upregulate let-7 miRNA levels and inducing differentiation in mouse embryonic stem cells (51). Lim et al. (52) screened an in-house library and found a benzopyaranylpyrazole-based compound as a primary hit molecule while Lightfoot et al. used a biophysical assay to identify 6-hydroxy-DL-DOPA and benzo[a]phenoxazine which inhibited the Lin28/let-7 interaction in vitro. The Sliz group developed a fluorescence polarization HTS and identified LI71 and TPEN, the latter one as a potent ZKD domain inhibitor (53). Despite the growing body of reported small molecule inhibitors of the Lin28/let-7 interaction, pharmacologic inhibition of LIN28 in vivo for targeted AML and LSC therapy has not been established. In addition, small molecule inhibitors with high specificity towards LIN28, inhibiting its activity, remain unexplored.

The present disclosure reports in vitro and in vivo inhibition of Lin28 and Lin28/let-7 by compounds of formula (I) or (II):

As described herein, compounds of formula (I) and (II) show Lin28/let-7 inhibitory activity in FRET assays in vitro and in LSCs and LSC-like Kasumi-1 cells. FRET assay was performed as previously described (51).

Similarly, compounds of formulae (I) and (II) demonstrate in vitro and in vivo inhibition of protein-RNA interactions, in particular Lin28/let-7 and PRPF31/U4.

The present disclosure provides a method of treating a cancer, comprising administering to a subject suffering from a cancer or displaying a symptom of a cancer, a compound or composition described herein. In some embodiments, the method comprises treating or ameliorating one or more symptoms of the cancer. In some embodiments, the cancer is a hematological cancer, e.g., acute myelogenous leukemia. In some embodiments, the methods comprise administering the compound or composition in an amount or according to a dosing regimen that has been determined to achieve inhibition of and/or reduced proliferation of a cancer cell. In some embodiments, the cancer cell comprises a cancer stem cell. In some embodiments, the cancer stem cell comprises a leukemic stem cell (LSC). In some embodiments, the methods comprise administering the compound or composition in an amount or according to a dosing regimen that has been determined to achieve inhibition of and/or reduced proliferation of a cancer cell, wherein the inhibition of and/or reduced proliferation of the cancer cell is evaluated using an assay shown in Examples 3 or 5, or a similar assay.

In some embodiments, the present disclosure provides a method of modulating splicing, the method comprising contacting a splicing-competent system with a compound as described herein.

In some embodiments, the present disclosure provides a method comprising:

contacting a splicing-competent system with a compound as described herein; and assessing in the system:
(i) presence or level of a splicing product (e.g., a spliced transcript);
(ii) expression or localization of an RNA; and/or
(iii) expression or folding of a polypeptide

In some embodiments, the present disclosure provides a method of modulating splicing in a splicing-competent system by contacting the system with a compound as described herein so that one or more of the following is observed:
(i) reduced splicing of an RNA;
(ii) altered expression or localization of an RNA; and/or
(iii) altered expression or folding of a polypeptide.

In some embodiments, the present disclosure provides a method comprising, contacting a splicing-competent system with a compound as described herein, wherein the compound is characterized in that when contacted with a cancer cell it reduces proliferation of the cancer cell relative to that observed in its absence. In some embodiments, splicing is reduced when the compound is present as compared with when it is absent. In some embodiments, the method further comprises assessing splicing in the system as compared with a reference condition. In some embodiments, the reference condition is absence of the compound. In some embodiments, the reference condition is presence of a control compound. In some embodiments, the reference condition is a historical condition. In some embodiments, the compound inhibits one or more attributes of a splicing machinery component and/or wherein the compound inhibits interaction between or among splicing machinery components. In some embodiments, the compound binds directly to one or more splicing machinery components, or complexes thereof. In some embodiments, the splicing machinery component is an RNA component. In some embodiments, the splicing machinery component is a polypeptide component. In some embodiments, the splicing machinery component is selected from RNA components, polypeptide components, and complexes thereof or therebetween. In some embodiments, the RNA component is or comprises a small nuclear RNA (snRNA). In some embodiments, the snRNA is selected from U1, U2, U4, U5, and U6. In some embodiments, the polypeptide component is or comprises an Sm polypeptide or an Lsm polypeptide. In some embodiments, the polypeptide component is selected from Prp3, Prp31, Prp4, CypH, 15.5K, Prp8, Brr2, Snu114, Prp6, Prp28, 40K, Dib1, Snu66, Sad1, and 27K. In some embodiments, the splicing machinery component comprises a Prp31 polypeptide. In some embodiments, the splicing machinery component comprises a U4 snRNA, a U6 snRNA and a Prp31 polypeptide. In some embodiments, the compound inhibits an interaction between: a U6 snRNA and a Prp31 polypeptide; or a U4 snRNA and a Prp31 polypeptide. In some embodiments, the compound inhibits an activity of a Prp31 polypeptide.

In some embodiments, the contacting occurs in vitro, ex vivo or in vivo. In some embodiments, the splicing-competent system is a cancer cell. In some embodiment, the splicing-competent cancer cell comprises a cancer stem cell. In some embodiments, the splicing-competent cancer stem cell comprises a leukemic stem cell (LSC).

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, l-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Comparable: As used herein, the term “comparable” refers to two or more agents, entities, situations, sets of conditions, that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Inhibitor: As used herein, the term “inhibitor” (or “inhibitory agent”) refers to an entity, condition, or event whose presence, level, or degree correlates with decreased level or activity of a target). In some embodiments, an inhibitory agent may be act directly (in which case it exerts its influence directly upon its target, for example by binding to the target); in some embodiments, an inhibitory agent may act indirectly (in which case it exerts its influence by interacting with and/or otherwise altering a regulator of the target, so that level and/or activity of the target is reduced). In some embodiments, an inhibitory agent is one whose presence or level correlates with a target level or activity that is reduced relative to a particular reference level or activity (e.g., that observed under appropriate reference conditions, such as presence of a known inhibitory agent, or absence of the inhibitory agent in question, etc).

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid. In some embodiments, a small molecule is a modulating agent (e.g., is an inhibiting/inhibitory agent or an activating agent). In some embodiments, a small molecule is biologically active. In some embodiments, a small molecule is detectable (e.g., comprises at least one detectable moiety). In some embodiments, a small molecule is a therapeutic agent. Those of ordinary skill in the art, reading the present disclosure, will appreciate that certain small molecule compounds described herein may be provided and/or utilized in any of a variety of forms such as, for example, crystal forms, salt forms, protected forms, pro-drug forms, ester forms, isomeric forms (e.g., optical and/or structural isomers), isotopic forms, etc. Those of skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form. Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms. Those of skill in the art will appreciate that certain small molecule compounds have structures that permit isotopic substitution (e.g., ²H or ³H for H; ¹¹C, ¹³C or ¹⁴C for 12C; ¹³N or ¹⁵N for 14N; ¹⁷O or ¹⁸O for 16O; ³⁶Cl for XXC; ¹⁸F for XXF; 131I for XXXI; etc). In some embodiments, such a small molecule may be utilized in accordance with the present disclosure in one or more isotopically modified forms, or mixtures thereof. In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Splicing component: Those skilled in the art, reading the present disclosure will appreciate that a “splicing component” is an agent or entity that participates in a splicing reaction. In some embodiments, a splicing component is or comprises a component of the spliceosome. In some embodiments, a splicing component is or comprises a splicing regulator. In some embodiments, a splicing component is or comprises an RNA, a polypeptide, and/or a complex thereof or therebetween. In some embodiments, one or more of a U1 snRNA, a U2 snRNA, a U4 snRNA, a U5 snRNA, a U6 snRNA, an Sm polypeptide, an Lsm polypeptide, a Prp3 polypeptide, a Prp31 polypeptide, a Prp4 polypeptide, a CypH polypeptide, a 15.5K polypeptide, a Prp8 polypeptide, a Brr2 polypeptide, a Snu114 polypeptide, a Prp6 polypeptide, a Prp28 polypeptide, a 40K polypeptide, a Dib1 polypeptide, a Snu66 polypeptide, a Sad1 polypeptide, or a 27K polypeptide, may be, or may be part of, a splicing component.

Splicing-competent system: Those skilled in the art, reading the present disclosure will appreciate that a “splicing-competent system” is a system that includes all components necessarily to accomplish one or more splicing events (e.g., of one or more particular RNAs). In some embodiments, a splicing-competent system may be an in vitro or ex vivo system. In some embodiments, a splicing-competent system may be or comprise one or more cells (e.g., in culture, in a tissue, or in an organism).

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “aliphatic” as used herein for compounds of formula (I) refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”)

Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₈ hydrocarbon or a bicyclic C₇-C₁₀ hydrocarbon that is completely saturated or that contains one or more units of unsaturation, but which is not aromatic. Suitable aliphatic groups include, but are not limited to, linear or branched, substituted or unsubstituted alkyl, alkenyl, alkynyl, alkylene, alkenylene, alkynylene groups and hybrids thereof.

As described herein, compounds of formula (I) may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

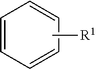

refers to at least

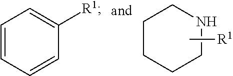

refers to at least

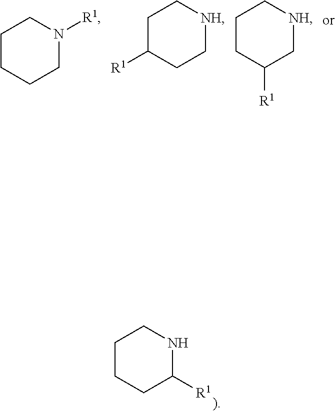

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)(NH)R^{∘}; —(CH₂)₀₋₄S(O)₂₀R^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), a 5- to 6-membered saturated, partially unsaturated, or aryl ring having 0-4 heteroatoms independently selected from nitrogen, oxygen, or sulfur, or an 8- to 10-membered bicyclic aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, -NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

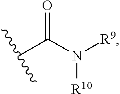

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

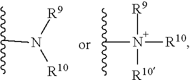

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

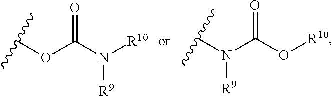

wherein R⁹ and R¹⁰ independently represent hydrogen or a hydrocarbyl group.

The term “carbocyclylalkyl”, as used herein, refers to an alkyl group substituted with a carbocycle group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

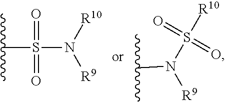

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

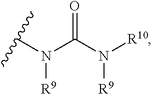

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers).

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples, which are included merely for purposes of illustration of certain aspects and embodiments of the present invention, and are not intended to limit the invention.

### Example 1: Synthesis

General Experimental Methods. All reactions were carried out under an argon atmosphere unless otherwise specified. Tetrahydrofuran (THF) was distilled from benzoquinone ketyl radical under an argon atmosphere. Dichloromethane and triethylamine were distilled from calcium hydride under an argon atmosphere. All other solvents and reagents were purified according to literature procedures or purchased from Sigma-Aldrich, Acros, Oakwood and Fisher Scientific Co. ¹H NMR spectra were recorded at 400 or 500 MHz and are reported relative to deuterated solvent signals. Data for ¹H NMR spectra are reported as follows: chemical shift (δ ppm), multiplicity, coupling constant (Hz), and integration. Splitting patterns are designated as follows: s, singlet; d, doublet; t, triplet; q, quartet; m, multiplet; and br, broad. ¹³C NMR spectra were recorded at 100 or 125 MHz. Data for ¹³C NMR spectra are reported in terms of chemical shift. The chemical shifts are reported in parts per million (ppm, δ). Thin-layer chromatography (TLC) was carried out using precoated silica gel sheets. Visual detection was performed using potassium permanganate or ceric ammonium nitrate stains. Flash chromatography was performed using SilicaFlash P60 (60 A, 40-63 μm) silica gel with compressed air.

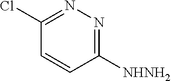

### 3-Chloro-6-hydrazineylpyridazine

To a solution of 3,6-dichloropyridazine (400 mg, 2.686 mmol) in EtOH (8 mL) was added hydrazine monohydrate (148 mg, 2.954 mmol) and the mixture was stirred at 100° C. for 3 h. After the mixture was cooled to 23° C., the resulting solid was collected and washed with Et₂O. The mother liquor was concentrated and the precipitate was washed with Et₂O. The combined solid was washed with dichloromethane to obtain the desired product (pale yellow, 320.2 mg, 2.216 mmol, 82%) and used for the next step without further purification. ¹H NMR (400 MHz, DMSO-d₆) δ 8.24 (br s, 1H), 7.41 (d, J=9.6 Hz, 1H), 7.09 (d, J=9.2 Hz, 1H), 4.37 (br s, 2H); ¹³C NMR (100 MHz, DMSO-d₆) δ 161.8, 145.4, 128.7, 116.1. Spectroscopic data match the literature data. [Ref: Heterocycles, 2009, 78 (4) 961-975]

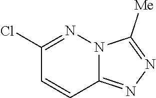 [SMILES: Cc1nnc2ccc(Cl)nn12]

### 6-Chloro-3-methyl-[1,2,4]triazolo[4,3-b]pyridazine

A mixture of 3-chloro-6-hydrazineylpyridazine (300 mg, 2.075 mmol) in AcOH (1.5 mL) was heated at 100° C. for 2 h. After the reaction mixture was cooled to 23° C., it was diluted with water and extracted with EtOAc. The combined organic layer was washed with sat. NaHCO₃ solution and brine, dried over anhydrous MgSO₄, filtered and concentrated under reduced pressure. The resulting crude off-white solid (238.5 mg, 68%) was used for the next step without further purification. ¹H NMR (400 MHz, CDCl₃) δ 8.04 (d, J=9.6 Hz, 1H), 7.09 (d, J=9.6 Hz, 1H), 2.81 (s, 3H).

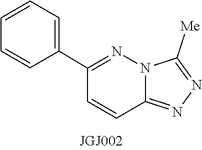 [SMILES: Cc1nnc2ccc(-c3ccccc3)nn12]

3-Methyl-6-phenyl-[1,2,4]triazolo[4,3-b]pyridazine, JGJ002. A mixture of 6-chloro-3-methyl-[1,2,4]triazolo[4,3-b]pyridazine (20 mg, 0.119 mmol), phenylboronic acid (14.5 mg, 0.119 mmol), K₂CO₃ (24.6 mg, 0.178 mmol) and Pd(PPh₃)₄ (13.6 mg, 0.012 mmol) in 1,4-dioxane (0.3 mL) and water (30 uL) was heated at 120° C. for 18 h. After the reaction mixture was cooled to 23° C., it was diluted with water and EtOAc. The organic layer was isolated and the aqueous layer was extracted with EtOAc. The combined organic layer was washed with brine, dried over anhydrous MgSO₄, filtered and concentrated under reduced pressure. The resulting crude residue was purified by flash column chromatography (dichloromethane:MeOH=10:1) to obtain the desired product JGJ002 (20.4 mg, 0.098 mmol, 82%) as an ivory solid. ¹H NMR (400 MHz, CDCl₃) δ. 8.13 (d, J=9.2 Hz, 1H), 7.98-8.01 (m, 2H), 7.54-7.56 (4H, m), 2.88 (s, 3H)¹³C NMR (100 MHz, CDCl₃) δ 153.4, 147.5, 143.4, 134.4, 130.9, 129.2, 127.2, 124.9, 118.8, 9.8.

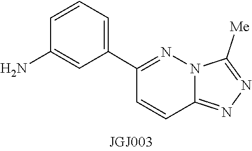 [SMILES: Cc1nnc2ccc(-c3cccc(N)c3)nn12]

3-(3-Methyl-[1,2,4]triazolo[4,3-b]pyridazin-6-yl)aniline, JGJ003. The reaction of 6-chloro-3-methyl-[1,2,4]triazolo[4,3-b]pyridazine (30 mg, 0.178 mmol), 3-nitrophenylboronic acid (35.6 mg, 0.214 mmol), K₂CO₃ (36.9 mg, 0.267 mmol) and Pd(PPh₃)₄ (20.6 mg, 0.018 mmol) in 1,4-dioxane (0.3 mL) and water (30 uL) afforded 3-methyl-6-(3-nitrophenyl)-[1,2,4]triazolo[4,3-b]pyridazine (19.7 mg, 0.077 mmol, 43%) using same procedure as described above. ¹H NMR (400 MHz, CDCl₃) δ. 8.86 (t, J=2.0 Hz, 1H), 8.39 (m, 2H), 8.24 (d, J=9.6 Hz, 1H), 7.71 (t, J=8.0 Hz, 1H), 7.62 (d, J=9.6 Hz, 1H), 2.91 (s, 3H)¹³C NMR (100 MHz, CDCl₃) δ 151.1, 148.8, 147.7, 143.2, 136.1, 132.8, 130.4, 125.8, 125.4, 122.2, 118.0, 9.9. Then a mixture of the nitro compound (19.4 mg, 0.076 mmol) and SnCl₂ (72.1 mg, 0.380 mmol) in EtOH (0.2 mL) was heated at reflux for 1 h. After the mixture was cooled to 23° C., it was filtered through Celite pad and washed with EtOAc. To the mixture was added sat. NaHCO₃ solution and it was extracted with EtOAc. The combined organic layer was washed with brine, dried over anhydrous MgSO₄, filtered and concentrated under reduced pressure. The resulting crude residue was purified by flash column chromatography (dichloromethane:MeOH=10:1) to obtain the desired product JGJ003 (10 mg, 0.044 mmol, 63%) as a pale yellow solid. ¹H NMR (400 MHz, CDCl₃) δ 8.10 (d, J=10.0 Hz, 1H), 7.51 (d, J=10.0 Hz, 1H), 7.26-7.32 (m, 3H), 6.83-6.86 (m, 1H), 2.86 (s, 3H); ¹³C NMR (100 MHz, CDCl₃) δ 153.5, 147.3 (two peaks overlapped), 143.4, 135.2, 130.0, 124.4, 119.1, 117.4, 117.2, 113.1, 9.7.

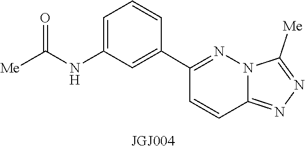 [SMILES: CC(=O)Nc1cccc(-c2ccc3nnc(C)n3n2)c1]

N-(3-(3-Methyl-[1,2,4]triazolo[4,3-b]pyridazin-6-yl)phenyl)acetamide, JGJ004. To a solution of 3-(3-methyl-[1,2,4]triazolo[4,3-b]pyridazin-6-yl)aniline (JGJ003, 20 mg, 0.088 mmol) in dichloro-methane (0.5 mL) was added trimethylamine (10.8 mg, 0.106 mmol) and acetyl chloride (7.6 mg, 0.099 mmol). The mixture was stirred at 23° C. for 6 h. To this mixture was added water and it was extracted with dichloromethane. The combined organic layer was washed with brine, dried over anhydrous MgSO₄, filtered and concentrated under reduced pressure. The crude residue was purified by flash column chromatography (dichloromethane:MeOH=6:1) to obtain the desired product JGJ004 (21.1 mg, 0.079 mmol, 89%) as an ivory solid. ¹H NMR (400 MHz, CDCl₃) δ 8.32 (s, 1H), 8.09 (d, J=9.6 Hz, 1H), 7.88 (br s, 1H), 7.70 (d, J=7.6 Hz, 1H), 7.65 (d, J=8.0 Hz, 1H), 7.54 (d, J=10.0 Hz, 1H), 7.48 (t, J=8.0 Hz, 1H), 2.86 (s, 3H), 2.25 (s, 3H). ¹³C NMR (125 MHz, CD₃OD) δ 172.8, 156.1, 149.9, 145.8, 141.8, 137.0, 131.5, 126.3, 124.8, 124.2, 122.6, 120.5, 24.8, 10.4.

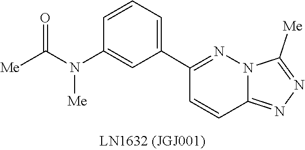 [SMILES: CC(=O)N(C)c1cccc(-c2ccc3nnc(C)n3n2)c1]

N-Methyl-N-(3-(3-methyl-[1,2,4]triazolo[4,3-b]pyridazin-6-yl)phenyl)acetamide, JGJ001. To a solution of N-(3-(3-methyl-[1,2,4]triazolo[4,3-b]pyridazin-6-yl)phenyl)acetamide (JGJ004, 16.5 mg, 0.062 mmol) was added NaH, 60% dispersion in mineral oil (5 mg, 0.124 mmol) at 0° C. and it was stirred for 30 min. Then iodomethane (17.5 mg, 0.124 mmol) was added and the reaction mixture was stirred at 23° C. for 2 h. After the reaction was completed, water was added and it was extracted with EtOAc. The combined organic layer was washed with brine, dried over anhydrous MgSO₄, filtered and concentrated under reduced pressure. The crude residue was purified by flash column chromatography (dichloromethane:MeOH=10:1) to obtain the desired product JGJ001 (9.8 mg, 0.035 mmol, 56%) as an ivory solid. ¹H NMR (500 MHz, CDCl₃) δ 8.17 (d, J=9.5 Hz, 1H), 7.95 (d, J=7.5 Hz, 1H), 7.88 (s, 1H), 7.62 (dd, J=8.0, 7.5 Hz, 1H), 7.54 (d, J=10.0 Hz, 1H), J=8.0 Hz, 1H), 3.35 (s, 3H), 2.89 (s, 3H), 1.94 (s, 3H); ¹³C NMR (125 MHz, CDCl₃) δ 170.3, 152.1, 147.6, 145.6, 143.3, 136.2, 130.7, 129.5, 126.4, 125.9, 125.4, 118.4, 37.3, 22.6, 9.9.

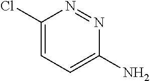

6-Chloropyridazin-3-amine. A mixture of 3,6-dichloropyridazine (200 mg, 2.342 mmol) and ammonium hydroxide (1.5 mL) in a sealed tube was heated 100° C. for 16 h. After the mixture was cooled to 23° C., dichloromethane was added and the precipitate was isolated and washed with dichloromethane to obtain the desired product (quant.) as a light yellow solid. ¹H NMR (400 MHz, DMSO-d₆) δ 7.32 (d, J=8.0 Hz, 1H), 6.81 (d, J=8.0 Hz, 1H), 6.59 (s, 2H).

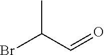

2-Bromopropionaldehyde. To a solution of propionaldehyde (2.91 mL, 40 mol) in dichloromethane (40 mL) was added dropwise bromine (2.05 mL, 40 mol) in dichloromethane (10 mL) at 0° C. over 1.5 h. The mixture was warmed to 23° C. and stirred for 30 min. After water was added to the reaction, the resulting organic layer was separated and washed with saturated sodium bicarbonate solution. The aqueous layer was extracted with dichloromethane (30 mL) and then the combined organic layer was washed with brine, dried over anhydrous MgSO₄, filtered and concentrated under reduced pressure. The crude product (dark yellow oil, quant.) was used for the next step without any purification. ¹H NMR (400 MHz, CDCl₃) δ 9.35 (br s, 1H), 4.34 (qd, J=6.8, 2.0 Hz, 1H), 1.75 (d, J=6.8 Hz, 3H). The spectroscopic data match the literature data. [Ref: Bull. Korean Chem. Soc. 2013, 34(1), 271-274.

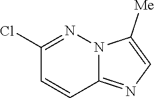 [SMILES: Cc1cnc2ccc(Cl)nn12]

6-Chloro-3-methylimidazo[1,2-b]pyridazine. A mixture of 6-chloropyridazin-3-amine (238.3 mg, 1.839 mmol) and 2-bromopropionaldehyde (crude, 503.9 mg, 3.679 mmol) in EtOH was heated at reflux for 4 h. After the mixture was cooled to 23° C., it was concentrated and extracted with EtOAc. The combined organic layer was washed with brine, dried over anhydrous MgSO₄, filtered and concentrated under reduced pressure. The crude residue was purified by flash column chromatography (n-Hex:EtOAc:MeOH=1:1:0.1) to obtain the desired product (55.2 mg, 0.329 mmol, 18%) as a light brown solid. ¹H NMR (400 MHz, CDCl₃) δ 7.87 (d, J=9.6 Hz, 1H), 7.56 (s, 1H), 6.99 (1H, J=9.6 Hz, 1H), 2.55 (s, 3H).

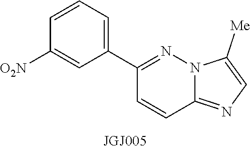 [SMILES: Cc1cnc2ccc(-c3cccc([N+](=O)[O-])c3)nn12]

3-Methyl-6-(3-nitrophenyl)imidazo[1,2-b]pyridazine, JGJ005. The reaction of 6-chloro-3-methylimidazolo[1,2-b]pyridazine (55.2 mg, 0.329 mmol), 3-nitrophenylboronic acid (60.5 mg, 0.362 mmol), K₂CO₃ (68.3 mg, 0.494 mmol) and Pd(PPh₃)₄ (38.1 mg, 0.033 mmol) in 1,4-dioxane (0.5 mL) and water (150 μL) afforded the desired product JGJ005 (61.9 mg, 0.244 mmol, 74%) as a yellow solid using the same procedure as described for JGJ002. ¹H NMR (500 MHz, CDCl₃) δ 8.88 (dd, J=2.0, 1.5 Hz, 1H), 8.38 (ddd, J=7.5, 1.5, 1.0 Hz, 1H), 8.35 (ddd, J=8.0, 2.0, 1.0 Hz, 1H), 8.07 (d, J=9.5 Hz, 1H), 7.73 (t, J=8.0 Hz, 1H), 7.67 (s, 1H), 7.50 (d, J=9.5 Hz, 1H), 2.67 (s, 3H); ¹³C NMR (125 MHz, CDCl₃) δ 148.8 (two peaks are overlapped), 138.1, 137.7, 133.3, 132.7, 130.0, 126.0, 125.8, 124.4, 122.0, 113.7, 8.8.

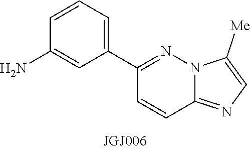 [SMILES: Cc1cnc2ccc(-c3cccc(N)c3)nn12]

3-(3-Methylimidazo[1,2-b]pyridazin-6-yl)aniline, JGJ006. A reaction of 3-methyl-6-(3-nitro-phenyl)imidazo[1,2-b]pyridazine (54.4 mg, 0.214 mmol) and SnCl₂ (202.8 mg, 1.070 mmol) in EtOH (0.5 mL) afforded the desired product JGJ006 (27.2 mg, 0.107 mmol, 50%) as a light yellow solid using the same procedure as described for JGJ003. ¹H NMR (400 MHz, CDCl₃) δ 7.92 (d, J=9.2 Hz, 1H), 7.56 (d, J=0.8 Hz, 1H), 7.38 (d, J=9.6 Hz, 1H), 7.28-7.34 (m, 3H), 6.79 (ddd, J=7.7, 2.0, 1.2 Hz, 1H), 3.87 (br s, 2H), 2.61 (d, J=0.8 Hz, 3H); ¹³C NMR (100 MHz, CDCl₃) δ 151.3, 147.0, 138.1, 137.0, 132.0, 129.8, 125.3, 125.1, 117.3, 116.5, 114.8, 113.3, 8.7.

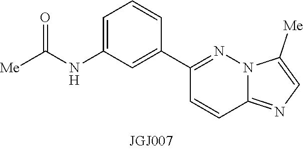 [SMILES: CC(=O)Nc1cccc(-c2ccc3ncc(C)n3n2)c1]

N-(3-(3-Methylimidazo[1,2-b]pyridazin-6-yl)phenyl)acetamide, JGJ007. The reaction of 3-(3-methylimidazo[1,2-b]pyridazin-6-yl)aniline (JGJ006, 23.3 mg, 0.104 mmol), triethylamine (12.6 mg, 0.125 mmol) and acetyl chloride (9 mg, 0.114 mmol) in dichloromethane (0.5 mL) afforded the desired product JGJ007 (16.5 mg, 0.067 mmol, 60%) as an ivory solid using the same procedure as described for JGJ004. ¹H NMR (400 MHz, CDCl₃) δ 8.44 (s, 1H), 8.21 (s, 1H), 7.89 (br s, 1H), 7.61-7.69 (m, 3H), 7.41 (t, J=8.0 Hz, 1H), 7.37 (br d, J=8.4 Hz, 1H), 2.57 (s, 3H), 2.22 (s, 3H); ¹³C NMR (100 MHz, CDCl₃) δ 168.8, 150.8, 138.9, 136.6, 132.2, 129.5, 125.2, 122.6, 121.2, 118.3, 114.6, 24.5, 8.7 (two low-field carbons not observed).

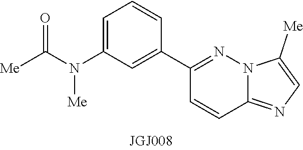 [SMILES: CC(=O)N(C)c1cccc(-c2ccc3ncc(C)n3n2)c1]

N-Methyl-N-(3-(3-methylimidazo[1,2-b]pyridazin-6-yl)phenyl)acetamide, JGJ008. The reaction of N-(3-(3-methylimidazo[1,2-b]pyridazin-6-yl)phenyl)acetamide (JGJ007, 26.4 mg, 0.099 mmol), NaH, 60% dispersion in mineral oil (8 mg, 0.199 mmol) and iodomethane (28.2 mg, 0.199 mmol) in dimethylformamide (DMF, 0.3 mL) afforded the desired product JGJ008 (17.5 mg, 0.062 mmol, 63%) as an ivory solid using the same procedure as described for JGJ001. ¹H NMR (400 MHz, CDCl₃) δ 8.00 (d, J=9.6 Hz, 1H), 7.96 (d, J=8.0 Hz, 1H), 7.89 (dd, J=2.0, 1.6 Hz, 1H), 7.62 (s, 1H), 7.58 (dd, J=8.0, 7.6 Hz, 1H), 7.43 (d, J=9.2 Hz, 1H), 7.32 (dd, J=7.6, 1.2 Hz, 1H), 3.34 (s, 3H), 2.64 (s, 3H), 1.95 (s, 3H); ¹³C NMR (125 MHz, CDCl₃) δ 170.5, 149.9, 145.4, 138.1, 137.8, 132.8, 130.4, 128.3, 126.2, 125.7, 125.6, 114.0, 37.2, 22.6, 8.8 (one low-field carbon not observed).

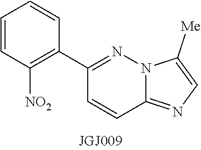 [SMILES: Cc1cnc2ccc(-c3ccccc3[N+](=O)[O-])nn12]

3-Methyl-6-(2-nitrophenyl)imidazo[1,2-b]pyridazine, JGJ009. The reaction of 6-chloro-3-methylimidazolo[1,2-b]pyridazine (67.1 mg, 0.400 mmol), 2-nitrophenylboronic acid (73.5 mg, 0.440 mmol), NaOH (48 mg, 1.201 mmol) and Pd(PPh₃)₄ (46.3 mg, 0.040 mmol) in THE (0.4 mL) and water (0.2 mL) at 80° C. afforded the desired product JGJ009 (16.3 mg, 0.064 mmol, 16%) as a yellow solid using the same procedure as described for JGJ002. ¹H NMR (400 MHz, CDCl₃) 8.02 (dd, J=8.0, 0.8 Hz, 1H), 7.99 (d, J=9.6 Hz, 1H), 7.75 (m, 1H), 7.64-7.70 (m, 2H), 7.63 (d, J=1.2 Hz, 1H), 7.10 (d, J=9.2 Hz, 1H), 2.54 (s, 3H); ¹³C NMR (100 MHz, CDCl₃) δ 149.6, 149.0, 137.7, 132.9, 132.8, 131.7, 131.4, 130.2, 125.6, 125.5, 124.7, 115.8, 8.6.

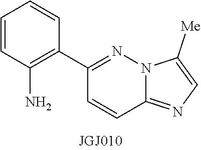 [SMILES: Cc1cnc2ccc(-c3ccccc3N)nn12]

2-(3-Methylimidazo[1,2-b]pyridazin-6-yl)aniline, JGJ010. The reaction of 6-chloro-3-methylimidazolo[1,2-b]pyridazine (25.4 mg, 0.152 mmol), 2-aminophenylboronic acid (22.8 mg, 0.167 mmol), K₂CO₃ (31.4 mg, 0.227 mmol) and Pd(PPh₃)₄ (17.5 mg, 0.015 mmol) in 1,4-dioxane (0.4 mL) and water (80 □L) at 110° C. afforded the desired product JGJ010 (26.2 mg, 0.117 mmol, 70%) as a pale yellow solid using the same procedure as described for JGJ002. ¹H NMR (400 MHz, CDCl₃) 7.97 (d, J=9.6 Hz, 1H), 7.57 (s, 1H), 7.67 (m, 1H), 7.42 (d, J=9.6 Hz, 1H), 7.24 (m, 1H), 6.82-6.87 (m, 2H), 2.59 (s, 3H); ¹³C NMR (100 MHz, CDCl₃) δ 152.8, 145.9, 137.3, 131.8, 130.7, 129.7, 125.6, 124.9, 118.6, 118.0, 117.4, 116.5, 8.8.

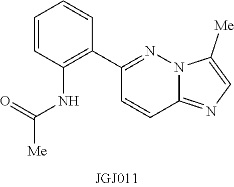 [SMILES: CC(=O)Nc1ccccc1-c1ccc2ncc(C)n2n1]

N-(2-(3-Methylimidazo[1,2-b]pyridazin-6-yl)phenyl)acetamide, JGJ011. The reaction of 2-(3-methylimidazo[1,2-b]pyridazin-6-yl)aniline (JGJ010, 39.4 mg, 0.176 mmol), triethylamine (21.3 mg, 0.211 mmol) and acetyl chloride (16.5 mg, 0.211 mmol) in dichloromethane (0.8 mL) afforded the desired product JGJ011 (35 mg, 0.131 mmol, 75%) as an ivory solid using the same procedure as described for JGJ004. ¹H NMR (400 MHz, CDCl₃) δ 10.57 (br s, NH), 8.47 (d, J=8.4 Hz, 1H), 7.99 (d, J=9.6 Hz, 1H), 7.61 (s, 1H), 7.60 (dd, J=8.0, 0.8 Hz, 1H), 7.44 (ddd, J=8.8, 7.2, 0.8 Hz, 1H), 7.34 (d, J=9.2 Hz, 1H), 7.20 (ddd, J=8.0, 7.2, 0.8 Hz, 1H), 2.60 (s, 3H), 2.17 (s, 3H); ¹³C NMR (100 MHz, CDCl₃) δ 168.1, 152.0, 137.3, 136.4, 132.8, 130.6, 129.5, 126.3, 124.6, 124.0, 123.5, 122.4, 116.7, 25.1, 8.9.

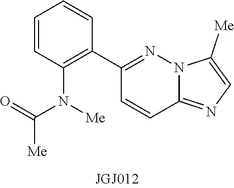 [SMILES: CC(=O)N(C)c1ccccc1-c1ccc2ncc(C)n2n1]

N-Methyl-N-(2-(3-methylimidazo[1,2-b]pyridazin-6-yl)phenyl)acetamide, JGJ012. The reaction of N-(2-(3-methylimidazo[1,2-b]pyridazin-6-yl)phenyl)acetamide (JGJ011, 19.1 mg, 0.072 mmol), sodium hydride (NaH, 60% dispersion in mineral oil, 5.7 mg, 0.143 mmol) and iodomethane (20.4 mg, 0.143 mmol) in dimethylformamide (DMF, 0.3 mL) afforded the desired product JGJ012 (12.8 mg, 0.046 mmol, 64%) as an ivory solid using the same procedure as described for JGJ001. ¹H NMR (400 MHz, CDCl₃) δ 7.98 (d, J=9.2 Hz, 1H), 7.66 (m, 1H), 7.60 (s, 1H) 7.52 (m, 2H), 7.34 (m, 1H), 7.10 (d, J=9.6 Hz, 1H), 3.01 (s, 3H), 2.54 (s, 3H), 1.90 (s, 3H); ¹³C NMR (100 MHz, CDCl₃) δ 170.9, 150.1, 142.5, 137.4, 134.5, 132.8, 131.0, 130.9, 130.7, 129.5, 128.7, 125.7, 116.0, 36.7, 22.7, 8.7.

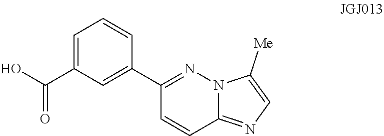 [SMILES: Cc1cnc2ccc(-c3cccc(C(=O)O)c3)nn12]

3-(3-Methylimidazo[1,2-b]pyridazin-6-yl)benzoic acid, JGJ013. The reaction of 6-chloro-3-methylimidazolo[1,2-b]pyridazine (50 mg, 0.299 mmol), 3-carboxyphenylboronic acid (54.5 mg, 0.328 mmol), K₂CO₃ (82.5 mg, 0.597 mmol) and Pd(PPh₃)₄ (34.5 mg, 0.030 mmol) in 1,4-dioxane (0.5 mL) and water (100 DL) afforded the desired product JGJ013 (32.4 mg, 0.128 mmol, 43%) as a white solid using the same procedure as described for JGJ002. ¹H NMR (400 MHz, CD₃OD) 8.73 (dd, J=1.6, 1.2 Hz, 1H), 8.25 (d, J=8.0 Hz, 1H), 8.16 (ddd, J=7.6, 1.6, 1.2 Hz, 1H), 8.03 (d, J=9.6 Hz, 1H), 7.75 (d, J=9.6 Hz, 1H), 7.62 (dd, J=8.0, 7.6 Hz, 1H), 7.58 (d, J=0.4 Hz, 1H), 2.63 (d, J=0.4 Hz, 3H).

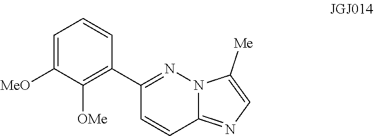 [SMILES: COc1cccc(-c2ccc3ncc(C)n3n2)c1OC]

6-(2,3-Dimethoxyphenyl)-3-methylimidazo[1,2-b]pyridazine, JGJ014. The reaction of 6-chloro-3-methylimidazolo[1,2-b]pyridazine (42 mg, 0.251 mmol), 2,3-dimethoxyphenylboronic acid (50.2 mg, 0.276 mmol), K₂CO₃ (52 mg, 0.376 mmol) and Pd(PPh₃)₄ (29 mg, 0.025 mmol) in 1,4-dioxane (0.5 mL) and water (100 DL) afforded the desired product JGJ014 (39.6 mg, 0.147 mmol, 59%) as an ivory solid using the same procedure as described for JGJ002. ¹H NMR (400 MHz, CDCl₃) 7.92 (d, J=9.6 Hz, 1H), 7.58 (s, 1H), 7.46 (d, J=9.2 Hz, 1H), 7.29 (dd, J=7.6, 0.8 Hz, 1H), 7.19 (t, J=8.0 Hz, 1H), 7.05 (ddd, J=8.0, 7.6, 0.8 Hz, 1H), 3.93 (s, 3H), 3.76 (s, 3H), 2.60 (s, 3H); ¹³C NMR (100 MHz, CDCl₃) δ 153.2, 150.7, 147.5, 138.0, 131.7, 131.1, 125.2, 124.4, 124.2, 122.2, 118.4, 113.6, 61.4, 56.0, 8.8.

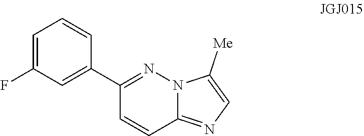 [SMILES: Cc1cnc2ccc(-c3cccc(F)c3)nn12]

6-(3-Fluorophenyl)-3-methylimidazo[1,2-b]pyridazine, JGJ015. The reaction of 6-chloro-3-methylimidazolo[1,2-b]pyridazine (51.5 mg, 0.307 mmol), 3-fluorophenylboronic acid (47.3 mg, 0.338 mmol), K₂CO₃ (63.7 mg, 0.461 mmol) and Pd(PPh₃)₄ (35.5 mg, 0.031 mmol) in 1,4-dioxane (0.5 mL) and water (100 DL) afforded the desired product JGJ015 (38.2 mg, 0.168 mmol, 55%) as an ivory solid using the same procedure as described for JGJ002. ¹H NMR (400 MHz, CDCl₃) 7.98 (d, J=9.2 Hz, 1H), 7.75 (m, 2H), 7.61 (s, 1H), 7.48 (m, 1H), 7.41 (d, J=9.2 Hz, 1H), 7.18 (m, 1H), 2.63 (s, 3H); ¹³C NMR (100 MHz, CDCl₃) δ 163.2 (d, J=244.9 Hz), 149.8 (d, J=2.6 Hz), 138.2, 138.1, 132.6, 130.5 (d, J=8.1 Hz), 125.5, 122.6 (d, J=2.9 Hz), 116.7 (d, J=21.2 Hz), 114.2, 113.9 (d, J=23.1 Hz), 8.7. (one low-field carbon not observed).

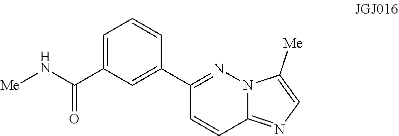 [SMILES: CNC(=O)c1cccc(-c2ccc3ncc(C)n3n2)c1]

N-Methyl-3-(3-methylimidazo[1,2-b]pyridazin-6-yl)benzamide, JGJ016. To a solution of JGJ013 (20.1 mg, 0.079 mmol) and methylamine hydrochloride (10.7 mg, 0.159 mmol) in dichloromethane (0.3 mL) and DMF (0.5 mL) was added hydroxybenzotriazole (HOBT, 16.1 mg, 0.159 mmol), (3-dimethylaminopropyl)-N′-ethylcarbodiimide hydrochloride, (EDC.HCl, 30.4 mg, 0.159 mmol) and N,N-diisopropylethylamine (DIPEA, 102.6 mg, 0.794 mmol). The mixture was stirred at 23° C. for 12 h. After water was added to the reaction, it was extracted with ethyl acetate (10 mL×3). The combined organic layer was washed with brine, dried over anhydrous MgSO₄, filtered and concentrated under reduced pressure. The crude residue was purified by flash column chromatography (dichloromethane:MeOH=6:1) to obtain the desired product JGJ016 (8.6 mg, 0.032 mmol, 41%) as a pale yellow solid. ¹H NMR (400 MHz, CDCl₃) δ 8.39 (t, J=1.6 Hz, 1H), 8.10 (dddd, J=8.0, 1.6, 1.2, 0.8 Hz, 1H), 7.91 (d, J=9.6 Hz, 1H), 7.86 (ddd, J=7.6, 1.6, 1.2 Hz, 1H), 7.58 (s, 1H), 7.55 (dd, J=8.0, 7.6 Hz, 1H), 7.41 (d, J=9.6 Hz, 1H), 6.75 (m, NH), 3.06 (d, J=4.8 Hz, 3H), 2.59 (d, J=0.4 Hz, 3H); ¹³C NMR (100 MHz, CDCl₃) δ 167.7, 153.3, 138.0, 136.3, 135.5, 132.3, 129.7, 129.2, 128.0, 125.7, 125.5, 125.4, 114.4, 26.9, 8.7.

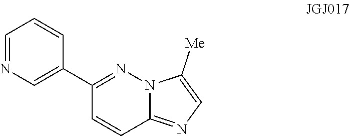 [SMILES: Cc1cnc2ccc(-c3cccnc3)nn12]

3-Methyl-6-(pyridin-3-yl)imidazo[1,2-b]pyridazine, JGJ017. The reaction of 6-chloro-3-methylimidazolo[1,2-b]pyridazine (58.8 mg, 0.351 mmol), 3-pyridineboronic acid (47.4 mg, 0.386 mmol), K₂CO₃ (72.7 mg, 0.526 mmol) and Pd(PPh₃)₄ (40.6 mg, 0.035 mmol) in 1,4-dioxane/water (5:1 v/v, 0.6 mL) afforded the desired product JGJ017 (37.2 mg, 0.177 mmol, 50%) as a pale yellow solid using the same procedure as described for JGJ002. ¹H NMR (400 MHz, CDCl₃) 9.20 (d, J=1.6 Hz, 1H), 8.69 (dd, J=4.8, 1.6 Hz, 1H), 8.29 (ddd, J=8.0, 2.0, 1.6 Hz, 1H), 7.98 (d, J=9.2 Hz, 1H), 7.60 (d, J=0.4 Hz, 1H), 7.42 (ddd, J=8.0, 4.8, 0.8 Hz, 1H), 7.41 (d, J=9.6 Hz, 1H), 2.60 (d, J=0.8 Hz, 3H); ¹³C NMR (100 MHz, CDCl₃) δ 150.6, 148.6, 148.2, 137.9, 134.2, 132.7, 131.6, 125.7, 125.5, 123.6, 113.7, 8.6.

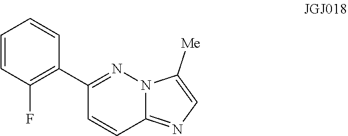 [SMILES: Cc1cnc2ccc(-c3ccccc3F)nn12]

6-(2-Fluorophenyl)-3-methylimidazo[1,2-b]pyridazine, JGJ018. The reaction of 6-chloro-3-methylimidazolo[1,2-b]pyridazine (27.5 mg, 0.164 mmol), 2-fluorophenylboronic acid (25.3 mg, 0.181 mmol), K₂CO₃ (34.0 mg, 0.246 mmol) and Pd(PPh₃)₄ (19.0 mg, 0.016 mmol) in 1,4-dioxane/water (5:1 v/v, 0.5 mL) afforded the desired product JGJ018 (18.1 mg, 0.080 mmol, 49%) as an ivory solid using the same procedure as described for JGJ002. ¹H NMR (400 MHz, CDCl₃) 7.96 (d, J=9.6 Hz, 1H), 7.91 (ddd, J=8.0, 7.6, 2.0 Hz, 1H), 7.60 (s, 1H), 7.43-7.49 (m, 2H), 7.30 (ddd, J=8.0, 7.6, 1.2 Hz, 1H), 7.21 (ddd, J=11.2, 8.4, 0.8 Hz, 1H), 2.61 (d, J=0.8 Hz, 3H); ¹³C NMR (100 MHz, CDCl₃) δ 160.4 (d, J=249.3 Hz), 148.2, 137.9, 132.2, 131.4 (d, J=8.5 Hz), 130.7 (d, J=2.6 Hz), 125.3, 124.7, 124.6 (d, J=3.6 Hz), 124.3 (d, J=11.7 Hz), 117.5 (d, J=7.9 Hz), 116.4 (d, J=22.2 Hz), 8.7.

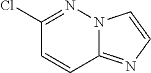

6-Chloroimidazo[1,2-b]pyridazine. To a solution of 6-chloropyridazin-3-amine (400 mg, 3.088 mmol) in EtOH (6 mL) and water (4 mL) was added bromoacetaldehyde diethyl acetal (930 μL, 6.175 mmol) and HBr (280 μL). The resulting mixture was heated at 103° C. overnight. After it was cooled to 23° C., the mixture was diluted water and extracted with EtOAc. The combined organic layer was washed with saturated NaHCO₃ solution, dried over anhydrous MgSO₄, filtered and concentrated under reduced pressure. The resulting crude residue was used for the next step without further purification. (Brown solid; 394.5 mg, 2.569 mmol, 83%)¹H NMR (400 MHz, CDCl₃) δ 7.92 (s, 1H), 7.90 (d, J=9.6 Hz, 1H), 7.76 (s, 1H), 7.04 (d, J=9.6 Hz, 1H); ¹³C NMR (100 MHz, CDCl₃) δ 146.9, 137.5, 134.4, 127.0, 118.9, 117.2.

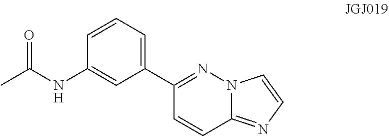 [SMILES: CC(=O)Nc1cccc(-c2ccc3nccn3n2)c1]

N-(3-(Imidazo[1,2-b]pyridazin-6-yl)phenyl)acetamide, JGJ019. The reaction of 6-chloro-imidazo[1,2-b]pyridazine (71.6 mg, 0.427 mmol), 3-aminophenylboronic acid (69.5 mg, 0.449 mmol), K₂CO₃ (88.6 mg, 0.641 mmol) and Pd(PPh₃)₄ (49.3 mg, 0.043 mmol) in 1,4-dioxane/water (5:1 v/v, 1.0 mL) afforded 3-(imidazo[1,2-b]pyridazin-6-yl)aniline (87.9 mg, 0.392 mmol, 92%) as a light yellow solid using the same procedure as described for JGJ002. Then the acetylation using the same procedure as described for JGJ004 gave the desired product JGJ019 (49.6 mg, 0.221 mmol, 69%) as an ivory solid. ¹H NMR (400 MHz, CDCl₃) δ 8.19 (s, 1H), 8.13 (br s, 1H), 7.96 (m, 2H), 7.76 (s, 1H), 7.61-7.65 (m, 2H), 7.43 (d, J=9.6 Hz, 1H), 7.39-7.43 (m, 1H), 2.22 (s, 3H); ¹³C NMR (100 MHz, CDCl₃) δ 168.9, 151.8, 138.9, 138.2, 136.1, 133.6, 129.7, 125.4, 122.7, 121.4, 118.4, 117.1, 116.7, 24.6.

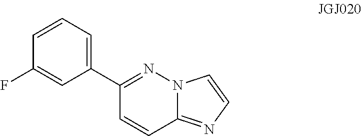 [SMILES: Fc1cccc(-c2ccc3nccn3n2)c1]

6-(3-Fluorophenyl)imidazo[1,2-b]pyridazine, JGJ020. The reaction of 6-chloroimidazo[1,2-b]pyridazine (50 mg, 0.326 mmol), 3-fluorophenylboronic acid (50.1 mg, 0.358 mmol), K₂CO₃ (67.5 mg, 0.488 mmol) and Pd(PPh₃)₄ (18.8 mg, 0.016 mmol) in 1,4-dioxane/water (5:1 v/v, 0.5 mL) afforded the desired product JGJ020 (36.9 mg, 0.173 mmol, 53%) as an ivory solid using the same procedure as described for JGJ002. ¹H NMR (400 MHz, CDCl₃) δ 7.96-7.99 (m, 2H), 7.77 (s, 1H), 7.62-7.68 (m, 2H), 7.42-7.46 (m, 1H), 7.39 (d, J=9.6 Hz, 1H), 7.14 (m, 1H); ¹³C NMR (100 MHz, CDCl₃) δ 163.1 (d, J=245.1 Hz), 150.4 (d, J=2.6 Hz), 137.5 (d, J=7.8 Hz), 134.2, 131.9 (d, J=9.8 Hz), 130.5 (d, J=8.1 Hz), 128.4 (d, J=12.1 Hz), 125.7, 122.5 (d, J=2.9 Hz), 116.8 (d, J=21.1 Hz), 115.7, 113.8 (d, J=23.2 Hz).

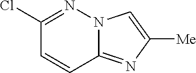 [SMILES: Cc1cn2nc(Cl)ccc2n1]

6-Chloro-2-methylimidazo[1,2-b]pyridazine. To a solution of 6-chloropyridazin-3-amine (100 mg, 0.772 mmol) in EtOH (2 mL) was added trimethylamine (78 mg, 0.772 mmol) and chloro-acetone (142.8 mg, 1.544 mmol) and the mixture was stirred at 120° C. overnight. After the mixture was cooled to 23° C., it was diluted with water and extracted with EtOAc. The combined organic layer was washed with brine, dried over anhydrous MgSO₄, filtered and concentrated under reduced pressure. The crude residue was purified by flash column chromatography (n-Hexane:EtOAc=1:1) to obtain the desired product (87.2 mg, 0.520 mmol, 67%) as off-white solid. ¹H NMR (400 MHz, CDCl₃) δ 7.72 (dd, J=9.2, 0.4 Hz, 1H), 7.65 (s, 1H), 6.93 (d, J=9.2 Hz, 1H), 2.44 (d, J=0.8 Hz, 3H); ¹³C NMR (100 MHz, CDCl₃) δ 145.8, 144.8, 137.0, 125.6, 117.9, 114.5, 14.7.

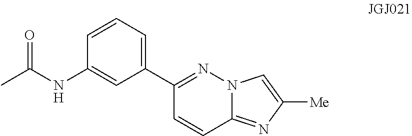 [SMILES: CC(=O)Nc1cccc(-c2ccc3nc(C)cn3n2)c1]

N-(3-(2-Methylimidazo[1,2-b]pyridazin-6-yl)phenyl)acetamide, JGJ021. The reaction of 6-chloro-2-methylimidazo[1,2-b]pyridazine (35.3 mg, 0.211 mmol), 3-aminophenylboronic acid (35.9 mg, 0.232 mmol), K₂CO₃ (43.7 mg, 0.316 mmol) and Pd(PPh₃)₄ (24.4 mg, 0.021 mmol) in 1,4-dioxane/water (5:1 v/v, 0.5 mL) afforded 3-(2-methylimidazo[1,2-b]pyridazin-6-yl)aniline (49.6 mg, quant.) as an pale yellow solid using the same procedure as described for JGJ002. Then the acetylation using the same procedure as described for JGJ004 gave the desired product JGJ021 (27.2 mg, 0.102 mmol, 46%) as an ivory solid. ¹H NMR (400 MHz, CDCl₃) δ 8.92 (s, 1H), 8.16 (s, 1H), 7.73 (d, J=9.6 Hz, 1H), 7.63 (m, 2H), 7.54 (d, J=7.6 Hz, 1H), 7.33 (t, J=8.0 Hz, 1H), 7.27 (d, J=10.0 Hz, 1H), 2.44 (s, 3H), 2.19 (s, 3H); ¹³C NMR (100 MHz, CDCl₃) δ 169.2, 150.7, 143.8, 139.0, 137.7, 136.1, 129.4, 123.9, 122.3, 121.1, 118.2, 115.7, 114.3, 24.4, 14.5.

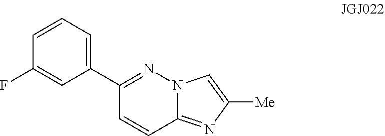 [SMILES: Cc1cn2nc(-c3cccc(F)c3)ccc2n1]

6-(3-Fluorophenyl)-2-methylimidazo[1,2-b]pyridazine, JGJ022. The reaction of 6-chloro-2-methylimidazo[1,2-b]pyridazine (21.4 mg, 0.128 mmol), 3-fluorophenylboronic acid (17.9 mg, 0.128 mmol), K₂CO₃ (26.5 mg, 0.192 mmol) and Pd(PPh₃)₄ (7.4 mg, 0.006 mmol) in 1,4-dioxane/water (5:1 v/v, 0.3 mL) afforded the desired product JGJ022 (13.7 mg, 0.060 mmol, 47%) as an ivory solid using the same procedure as described for JGJ002. ¹H NMR (400 MHz, CDCl₃) δ 7.89 (d, J=9.2 Hz, 1H), 7.78 (s, 1H), 7.65-7.70 (m, 2H), 7.43-7.49 (m, 1H), 7.38 (d, J=9.2 Hz, 1H), 7.16 (m, 1H), 2.52 (d, J=0.4 Hz, 3H); ¹³C NMR (100 MHz, CDCl₃) δ 163.2 (d, J=245.0 Hz), 149.8 (d, J=2.7 Hz), 144.5, 137.9 (d, J=8.0 Hz), 130.5 (d, J=8.2 Hz), 124.5, 122.5 (d, J=3.0 Hz), 116.7 (d, J=21.1 Hz), 115.3, 114.4, 113.9 (d, J=23.2 Hz), 14.8. (one low-field carbon not observed)

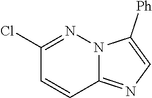 [SMILES: Clc1ccc2ncc(-c3ccccc3)n2n1]

6-Chloro-3-phenylimidazo[1,2-b]pyridazine. To a solution of 6-chloroimidazo[1,2-b]pyridazine (394.5 mg, 2.569 mmol) in DMF (6 mL) was added N-iodosuccinimide (635.8 mg, 2.826 mmol) and the mixture was stirred at 23° C. for 48 h. After the reaction was completed, it was vacuumed to remove the solvent. The residue was diluted with dichloromethane and washed with saturated Na₂S₂CO₃ solution. The organic layer was separated and washed with brine, dried over anhydrous MgSO₄, filtered and concentrated under reduced pressure to give 6-chloro-3-iodoimidazo[1,2-b]pyridazine in quantitative yield. Then a mixture of 6-chloro-3-iodoimidazo[1,2-b]pyridazine (107.2 mg, 0.326 mmol), phenylboronic acid (43.7 mg, 0.358 mmol), K₂CO₃ (54.0 mg, 0.391 mmol) and Pd(PPh₃)₄ (18.8 mg, 0.016 mmol) in 1,4-dioxane/water (5:1 v/v, 2 mL) was heated at 90° C. overnight. After the reactant was cooled to 23° C., it was diluted in water and extracted with EtOAc. The combined organic layer was washed with brine, dried over anhydrous MgSO₄, filtered and concentrated under reduced pressure. The crude residue was purified by flash column chromatography (n-Hexane:EtOAc=2:1) to obtain the desired product (28.4 mg, 0.124 mmol, 38%) as a pale yellow solid. ¹H NMR (400 MHz, CDCl₃) δ 8.06 (s, 1H), 8.03 (m, 2H), 7.98 (d, J=9.6 Hz, 1H), 7.52 (m, 2H), 7.39 (m, 1H), 7.08 (d, J=9.2 Hz, 1H); ¹³C NMR (100 MHz, CDCl₃) δ 146.8, 138.5, 133.1, 129.1, 128.7, 128.4, 127.6, 127.1, 126.8, 118.3.

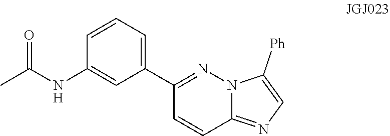 [SMILES: CC(=O)Nc1cccc(-c2ccc3ncc(-c4ccccc4)n3n2)c1]

N-(3-(3-Phenylimidazo[1,2-b]pyridazin-6-yl)phenyl)acetamide, JGJ023. The reaction of 6-chloro-3-phenylimidazo[1,2-b]pyridazine (15.5 mg, 0.068 mmol), 3-aminophenylboronic acid (11.5 mg, 0.074 mmol), K₂CO₃ (14.0 mg, 0.101 mmol) and Pd(PPh₃)₄ (3.9 mg, 0.003 mmol) in 1,4-dioxane/water (5:1 v/v, 0.2 mL) afforded 3-(3-phenylimidazo[1,2-b]pyridazin-6-yl)aniline (17.5 mg, 0.061 mmol, 91%) as an pale yellow solid using the same procedure as described for JGJ002. Then the acetylation using the same procedure as described for JGJ004 gave the desired product JGJ023 (10.9 mg, 0.033 mmol, 54%) as an ivory solid. ¹H NMR (400 MHz, CDCl₃) δ 8.18 (s, 1H), 8.12 (m, 2H), 8.04 (s, 1H), 7.99 (d, J=9.6 Hz, 1H), 7.93 (br s, 1H), 7.64-7.70 (m, 2H), 7.50 (m, 2H), 7.46 (d, J=9.6 Hz, 1H), 7.35-7.44 (m, 2H), 2.22 (s, 3H); ¹³C NMR (100 MHz, CDCl₃) δ 168.7, 151.1, 138.8, 136.4, 133.0, 129.6, 128.8, 128.7, 128.6, 127.9, 126.8, 125.8, 122.7, 121.3, 118.3, 115.6, 24.6. (one low-field carbon not observed)

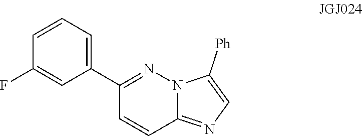 [SMILES: Fc1cccc(-c2ccc3ncc(-c4ccccc4)n3n2)c1]

6-(3-Fluorophenyl)-3-phenylimidazo[1,2-b]pyridazine, JGJ024. The reaction of 6-chloro-3-phenylimidazo[1,2-b]pyridazine (12.9 mg, 0.056 mmol), 3-fluorophenylboronic acid (8.6 mg, 0.062 mmol), K₂CO₃ (11.7 mg, 0.084 mmol) and Pd(PPh₃)₄ (3.2 mg, 0.003 mmol) in 1,4-dioxane/water (5:1 v/v, 0.2 mL) afforded the desired product JGJ024 (9.5 mg, 0.033 mmol, 58%) as an ivory solid using the same procedure as described for JGJ002. ¹H NMR (400 MHz, CDCl₃) δ 8.10-8.14 (m, 4H), 7.72-7.79 (m, 2H), 7.48-7.56 (m, 4H), 7.42 (m, 1H), 7.20 (m, 1H); ¹³C NMR (100 MHz, CDCl₃) δ 163.2 (d, J=245.0 Hz), 150.5 (d, J=2.7 Hz), 137.8 (d, J=7.8 Hz), 133.0, 130.6 (d, J=8.2 Hz), 129.1, 128.8, 128.4, 128.1, 127.1, 126.9, 126.1, 122.7 (d, J=2.9 Hz), 117.0 (d, J=21.2 Hz), 115.3, 114.0 (d, J=23.2 Hz).

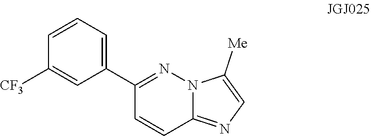 [SMILES: Cc1cnc2ccc(-c3cccc(C(F)(F)F)c3)nn12]

3-Methyl-6-(3-(trifluoromethyl)phenyl)imidazo[1,2-b]pyridazine, JGJ025. The reaction of 6-chloro-3-methylimidazolo[1,2-b]pyridazine (35.9 mg, 0.214 mmol), 3-trifluoromethylphenylboronic acid (42.7 mg, 0.225 mmol), K₂CO₃ (44.4 mg, 0.321 mmol) and Pd(PPh₃)₄ (12.4 mg, 0.011 mmol) in 1,4-dioxane/water (5:1 v/v, 0.4 mL) afforded the desired product JGJ018 (29.2 mg, 0.105 mmol, 49%) as a white solid using the same procedure as described for JGJ002. ¹H NMR (400 MHz, CDCl₃) δ 8.27 (s, 1H), 8.20 (d, J=8.0 Hz, 1H), 8.09 (d, J=9.2 Hz, 1H), 7.76 (d, J=8.0 Hz, 1H), 7.65-7.69 (m, 2H), 7.51 (d, J=9.2 Hz, 1H), 2.66 (s, 3H); ¹³C NMR (100 MHz, CDCl₃) δ 149.8, 136.7, 132.6, 132.1 (d, J=9.8 Hz), 131.5 (q, J=32.4 Hz), 130.2, 129.5, 128.4 (d, J=12.0 Hz), 126.4 (q, J=3.5 Hz), 125.7, 123.9 (q, J=270.8 Hz), 123.8 (q, J=3.8 Hz), 114.1, 8.7. (¹³C NMR will be taken again due to existence of some impurity)

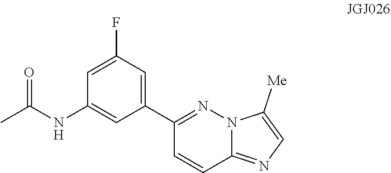 [SMILES: CC(=O)Nc1cc(F)cc(-c2ccc3ncc(C)n3n2)c1]

N-(3-Fluoro-5-(3-methylimidazo[1,2-b]pyridazin-6-yl)phenyl)acetamide, JGJ026. The reaction of 6-chloro-3-methylimidazo[1,2-b]pyridazine (35.3 mg, 0.211 mmol), 3-fluoro-5-aminophenylboronic acid (34.3 mg, 0.221 mmol), K₂CO₃ (43.7 mg, 0.316 mmol) and Pd(PPh₃)₄ (12.2 mg, 0.011 mmol) in 1,4-dioxane/water (5:1 v/v, 0.4 mL) afforded 3-fluoro-5-(3-methylimidazo[1,2-b]pyridazin-6-yl)aniline (25 mg, 0.103 mmol, 49%) as a pale yellow solid using the same procedure as described for JGJ002. Then the acetylation using the same procedure as described for JGJ004 gave the desired product JGJ026 (8 mg, 0.028 mmol, 28%) as a pale yellow solid. ¹H NMR (400 MHz, CDCl₃) δ 8.38 (br s, 1H), 8.00 (d, J=9.2 Hz, 1H), 7.89 (s, 1H), 7.65 (d, J=9.2 Hz, 1H), 7.60 (s, 1H), 7.43 (s, 1H), 7.41 (s, 1H), 2.60 (s, 3H), 2.24 (s, 3H);

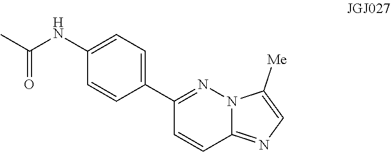 [SMILES: CC(=O)Nc1ccc(-c2ccc3ncc(C)n3n2)cc1]

N-(4-(3-Methylimidazo[1,2-b]pyridazin-6-yl)phenyl)acetamide, JGJ027. The reaction of 6-chloro-3-methyllimidazo[1,2-b]pyridazine (35.3 mg, 0.211 mmol), 4-aminophenylboronic acid (38.4 mg, 0.221 mmol), K₂CO₃ (43.7 mg, 0.316 mmol) and Pd(PPh₃)₄ (12.2 mg, 0.011 mmol) in 1,4-dioxane/water (5:1 v/v, 0.4 mL) afforded 4-(3-methylimidazo[1,2-b]pyridazin-6-yl)aniline (31.4 mg, 0.140 mmol, 66%) as a light yellow solid using the same procedure as described for JGJ002. Then the acetylation using the same procedure as described for JGJ004 gave the desired product JGJ026 (7.2 mg, 0.027 mmol, 19%) as an ivory solid. ¹H NMR (400 MHz, CDCl₃) δ 7.99 (d, J=8.8 Hz, 2H), 7.95 (d, J=9.2 Hz, 1H), 7.68 (d, J=8.4 Hz, 2H), 7.58 (s, 1H), 7.47 (br s, 1H), 7.43 (d, J=9.6 Hz, 1H), 2.62 (s, 3H), 2.23 (s, 3H);

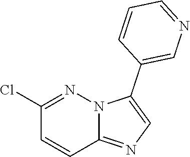 [SMILES: Clc1ccc2ncc(-c3cccnc3)n2n1]

6-Chloro-3-(pyridin-3-yl)imidazo[1,2-b]pyridazine. The reaction of 6-chloro-3-iodoimidazo[1,2-b]pyridazine (82.6 mg, 0.297 mmol), pyridine-3-boronic acid (40 mg, 0.325 mmol), K₂CO₃ (61.3 mg, 0.443 mmol) and Pd(PPh₃)₄ (17.1 mg, 0.015 mmol) in 1,4-dioxane/water (5:1 v/v, 1 mL) at 100° C. afforded the desired product (41.5 mg, 0.180, 61%) as a pale yellow solid using the same procedure as described for 6-chloro-3-phenylimidazo[1,2-b]pyridazine. ¹H NMR (400 MHz, CDCl₃) δ 9.21 (s, 1H), 8.62 (s, 1H), 8.40 (m, 1H), 8.11 (s, 1H), 7.98 (d, J=9.6 Hz, 1H), 7.43 (dd, J=7.6, 0.8 Hz, 1H), 7.12 (d, J=9.2 Hz, 1H); ¹³C NMR (100 MHz, CDCl₃) δ 149.0, 147.6, 147.2, 139.1, 133.6, 133.5, 127.4, 126.0, 124.3, 123.6, 118.9.

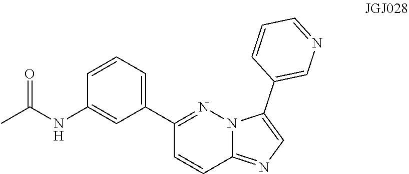 [SMILES: CC(=O)Nc1cccc(-c2ccc3ncc(-c4cccnc4)n3n2)c1]

N-(3-(3-(Pyridin-3-yl)imidazo[1,2-b]pyridazin-6-yl)phenyl)acetamide, JGJ028. The reaction of 6-chloro-3-(pyridin-3-yl)imidazo[1,2-b]pyridazine (41.5 mg, 0.180 mmol), 3-aminophenyl-boronic acid (30.7 mg, 0.198 mmol), K₂CO₃ (37.3 mg, 0.270 mmol) and Pd(PPh₃)₄ (10.4 mg, 0.009 mmol) in 1,4-dioxane/water (5:1 v/v, 0.4 mL) afforded 3-(3-(pyridin-3-yl)imidazo[1,2-b]pyridazin-6-yl)aniline (50.0 mg, 0.174 mmol, 96%) as an ivory solid using the same procedure as described for JGJ002. Then the acetylation using the same procedure as described for JGJ004 gave the desired product JGJ028 (18.2 mg, 0.055 mmol, 32%) as a pale yellow solid. ¹H NMR (400 MHz, CD₃OD) δ 9.29 (d, J=1.2 Hz, 1H), 8.60 (ddd, J=8.0, 2.0, 1.6 Hz, 1H), 8.49 (d, J=4.0 Hz, 1H), 8.25 (dd, J=2.0, 1.6 Hz, 1H), 8.18 (s, 1H), 8.02 (d, J=9.6 Hz, 1H), 7.68 (d, J=9.6 Hz, 1H), 7.60-7.65 (m, 2H), 7.55 (dd, J=8.0, 4.8 Hz, 1H), 7.37 (t, J=8.0 Hz, 1H), 2.16 (s, 3H); ¹³C NMR (100 MHz, CD₃OD) δ 172.1, 153.6, 149.2, 148.0, 141.5, 141.2, 137.1, 135.9, 134.1, 130.8, 127.1, 127.0, 126.9, 125.7, 123.8, 123.0, 119.5, 118.6, 24.3.

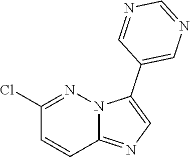 [SMILES: Clc1ccc2ncc(-c3cncnc3)n2n1]

6-Chloro-3-(pyrimidin-5-yl)imidazo[1,2-b]pyridazine. The reaction of 6-chloro-3-iodoimidazo[1,2-b]pyridazine (83.6 mg, 0.299 mmol), pyrimidine-5-boronic acid (40.8 mg, 0.329 mmol), K₂CO₃ (62 mg, 0.449 mmol) and Pd(PPh₃)₄ (17.3 mg, 0.015 mmol) in 1,4-dioxane/water (5:1 v/v, 1 mL) at 100° C. afforded the desired product (9.8 mg, 0.042 mmol, 14%) as a pale yellow solid using the same procedure as described for 6-chloro-3-phenylimidazo[1,2-b]pyridazine. ¹H NMR (400 MHz, CDCl₃) δ 9.42 (s, 2H), 9.23 (s, 1H), 8.18 (s, 1H), 8.04 (d, J=9.6 Hz, 1H), 7.20 (d, J=9.6 Hz, 1H); ¹³C NMR (100 MHz, CDCl₃) δ 157.7, 154.0, 147.7, 133.7, 132.1, 128.5, 127.7, 123.0, 119.8.

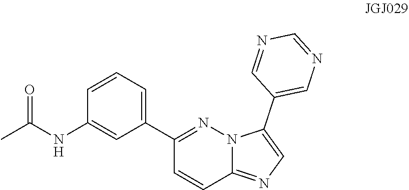 [SMILES: CC(=O)Nc1cccc(-c2ccc3ncc(-c4cncnc4)n3n2)c1]

N-(3-(3-(Pyrimidin-5-yl)imidazo[1,2-b]pyridazin-6-yl)phenyl)acetamide, JGJ029. The reaction of 6-chloro-3-(pyrimidin-5-yl)imidazo[1,2-b]pyridazine (9.8 mg, 0.042 mmol), 3-aminophenylboronic acid (7.2 mg, 0.047 mmol), K₂CO₃ (8.8 mg, 0.064 mmol) and Pd(PPh₃)₄ (4.9 mg, 0.004 mmol) in 1,4-dioxane/water (5:1 v/v, 0.2 mL) afforded 3-(3-(pyridin-3-yl)imidazo[1,2-b]pyridazin-6-yl)aniline (6.7 mg, 0.023 mmol, 55%) as a light yellow solid using the same procedure as described for JGJ002. Then the acetylation using the same procedure as described for JGJ004 gave the desired product JGJ029 (5.1 mg, 0.015 mmol, 67%) as an ivory solid. ¹H NMR (400 MHz, CDCl₃+5% v/v CD₃OD) δ 9.58 (s, 2H), 9.18 (s, 1H), 8.23 (s, 1H), 8.19 (d, J=9.6 Hz, 1H), 8.10 (s, 1H), 7.99 (d, J=8.0 Hz, 1H) 7.65 (d, J=9.2 Hz, 1H), 7.63 (d, J=8.0 Hz, 1H), 7.46 (dd, J=8.4, 7.6 Hz, 1H), 2.19 (s, 3H); ¹³C NMR (125 MHz, CDCl₃+5% v/v CD₃OD) δ 169.7, 156.8, 153.9, 152.5, 139.6, 134.7, 131.9, 129.9, 125.9, 123.7, 122.4, 122.2, 122.1, 118.0, 117.7, 117.6, 24.0.

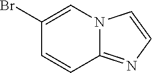

6-Bromoimidazo[1,2-a]pyridine. To a solution of 2-amino-5-bromopyridine (500 mg, 2.89 mmol) in EtOH (6 mL) and water (4 mL) was added bromoacetaldehyde diethyl acetal (870 μL, 5.78 mmol) and HBr (260 μL) at 23° C. The resulting mixture was heated at 103° C. overnight. After it was cooled to 23° C., the mixture was diluted in water and extracted with EtOAc. The combined organic layer was washed with saturated NaHCO₃ solution, dried over anhydrous MgSO₄, filtered and concentrated under reduced pressure. The resulting crude residue was used for the next step without further purification. (Brown solid; 331.7 mg, 1.68 mmol, 58%)¹H NMR (400 MHz, CDCl₃) δ 8.09 (dd, J=2.0, 0.8 Hz, 1H), 7.46 (d, J=0.8 Hz, 1H), 7.39 (s, 1H), 7.32 (d, J=9.6 Hz, 1H), 7.00 (dd, J=9.6, 2.0 Hz, 1H); ¹³C NMR (100 MHz, CDCl₃) δ 143.2, 133.8, 127.3, 125.4, 117.8, 112.3, 106.5.

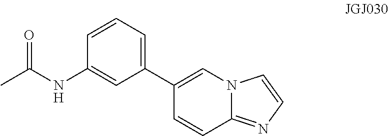 [SMILES: CC(=O)Nc1cccc(-c2ccc3nccn3c2)c1]

N-(3-(Imidazo[1,2-a]pyridin-6-yl)phenyl)acetamide, JGJ030. The reaction of 6-bromoimidazo[1,2-a]pyridine (50 mg, 0.254 mmol), 3-aminophenylboronic acid (43.3 mg, 0.279 mmol), K₂CO₃ (52.6 mg, 0.381 mmol) and Pd(PPh₃)₄ (29.3 mg, 0.025 mmol) in 1,4-dioxane/water (5:1 v/v, 1 mL) afforded 3-(imidazo[1,2-a]pyridin-6-yl)aniline (22.3 mg, 0.107 mmol, 42%) as an ivory solid using the same procedure as described for JGJ002. Then the acetylation using the same procedure as described for JGJ004 gave the desired product JGJ030 (13.6 mg, 0.054 mmol, 51%) as a white solid. ¹H NMR (400 MHz, CD₃OD) δ 8.68 (s, 1H), 7.89-7.94 (m, 2H), 7.56-7.62 (m, 3H), 7.51 (ddd, J=7.6, 2.0, 1.2 Hz, 1H), 7.35-7.43 (m, 2H), 2.16 (s, 3H); ¹³C NMR (100 MHz, CD₃OD) δ 170.3, 139.2, 137.4, 132.1, 129.1, 126.7, 125.7, 123.8, 122.1, 119.1, 118.0, 115.8, 113.5, 22.4. (one low-field carbon not observed)

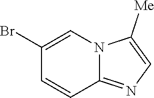 [SMILES: Cc1cnc2ccc(Br)cn12]

6-Bromo-3-methylimidazo[1,2-a]pyridine. A mixture of 2-amino-5-bromopyridine (200 mg, 1.156 mmol) and 2-bromopropionaldehyde (purity >95%, 318 mg, 2.312 mmol) in EtOH (5 mL) was heated at reflux overnight. After the mixture was cooled to 23° C., it was concentrated and extracted with EtOAc. The combined organic layer was washed with brine, dried over anhydrous MgSO₄, filtered and concentrated under reduced pressure. The crude residue was purified by flash column chromatography (n-Hexane:EtOAc=3:2) to obtain the desired product (86.9 mg, 0.412 mmol, 36%) as a white solid. ¹H NMR (400 MHz, CDCl₃) δ 8.00 (d, J=1.2 Hz, 1H), 7.49 (d, J=9.2 Hz, 1H), 7.40 (s, 1H), 7.20 (dd, J=9.6, 2.0 Hz, 1H), 2.46 (s, 3H); ¹³C NMR (100 MHz, CDCl₃) δ 143.5, 132.1, 126.5, 123.0, 120.3, 118.3, 106.9. 9.0.

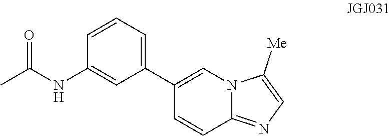 [SMILES: CC(=O)Nc1cccc(-c2ccc3ncc(C)n3c2)c1]

N-(3-(3-Methylimidazo[1,2-a]pyridin-6-yl)phenyl)acetamide, JGJ031. The reaction of 6-bromo-3-methylimidazo[1,2-a]pyridine (35 mg, 0.166 mmol), 3-aminophenylboronic acid (28.3 mg, 0.182 mmol), K₂CO₃ (34.4 mg, 0.249 mmol) and Pd(PPh₃)₄ (9.6 mg, 0.008 mmol) in 1,4-dioxane/water (5:1 v/v, 0.3 mL) afforded 3-(3-methylimidazo[1,2-a]pyridin-6-yl)aniline (28.1 mg, 0.106 mmol, 64%) as an ivory solid using the same procedure as described for JGJ002. Then the acetylation using the same procedure as described for JGJ004 gave the desired product JGJ031 (15.8 mg, 0.060 mmol, 56%) as an ivory solid. ¹H NMR (400 MHz, CDCl₃) δ 8.30 (br s, 1H), 8.12 (s, 1H), 7.87 (s, 1H), 7.65 (d, J=8.0 Hz, 1H), 7.54 (d, J=8.0 Hz, 1H), 7.36-7.43 (m, 3H), 7.27 (m, 1H), 2.49 (s, 3H), 2.23 (s, 3H);

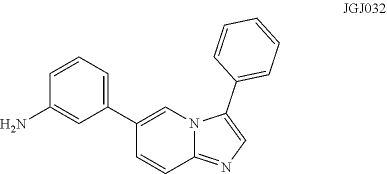 [SMILES: Nc1cccc(-c2ccc3ncc(-c4ccccc4)n3c2)c1]

3-(3-Phenylimidazo[1,2-a]pyridin-6-yl)aniline, JGJ032. To a mixture of 2-amino-5-bromo-pyridine (100 mg, 0.508 mmol), 3-aminophenylboronic acid (76.5 mg, 0.558 mmol), triphenylphosphine (26.6 mg, 0.102 mmol) and K₂CO₃ (140.3 mg, 1.015 mmol) in toluene: EtOH mixture (2:1 v/v, 1.7 mL) in a microwave tube was added Pd(OAc)₂ (11.4 mg, 0.059 mmol) and charged with argon. The mixture was sealed with a silicon septum and irradiated in microwave at 140° C. with stirring for 30 min. After the mixture had been allowed to cool to 23° C., bromobenzene (119.5 mg, 0.761 mmol) was injected into the tube by syringe and the mixture was again subjected to microwave irradiation at 140° C. with stirring for 2.5 h. The reaction vessel was cooled to 23° C. and the mixture was diluted with water and extracted with dichloromethane. The combined organic layer was dried over anhydrous MgSO₄, filtered and concentrated under reduced pressure. The crude residue was purified by flash column chromatography (n-Hexane:EtOAc: MeOH=1:1:0.1) to obtain the desired product (28.8 mg, 0.101 mmol, 20%) as a pale yellow solid. ¹H NMR (400 MHz, CDCl₃) δ 8.46 (s, 1H), 7.83 (d, J=9.2 Hz, 1H), 7.73 (s, 1H), 7.45-7.61 (m, 6H), 7.23 (d, J=8.0 Hz, 1H), 6.90 (d, J=8.0 Hz, 1H), 6.81 (t, J=2.0 Hz, 1H), 6.71 (m, 1H);

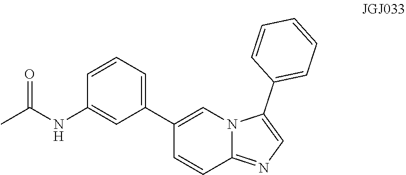 [SMILES: CC(=O)Nc1cccc(-c2ccc3ncc(-c4ccccc4)n3c2)c1]

N-(3-(3-Phenylimidazo[1,2-a]pyridin-6-yl)phenyl)acetamide, JGJ033. The reaction of 3-(3-phenylimidazo[1,2-a]pyridin-6-yl)aniline (JGJ032, 22.8 mg, 0.080 mmol), triethylamine (12.1 mg, 0.120 mmol) and acetyl chloride (9.4 mg, 0.120 mmol) in dichloromethane (2 mL) afforded the desired product JGJ033 (12.2 mg, 0.037 mmol, 47%) as an ivory solid using the same procedure as described for JGJ004. ¹H NMR (400 MHz, CD₃OD) δ 8.48 (s, 1H), 7.80 (dd, J=2.0, 1.6 Hz, 1H), 7.73 (s, 1H), 7.51-7.65 (m, 7H), 7.43 (m, 1H), 7.35 (dd, J=8.0 Hz, 1H), 7.27 (m, 1H), 2.12 (s, 3H); ¹³C NMR (100 MHz, CD₃OD) δ 170.3, 139.2, 137.4, 131.2, 129.2, 129.0, 128.4, 128.2, 127.7, 127.1, 126.5, 125.6, 122.0, 120.5, 119.0, 117.8, 116.5, 22.4. (one low-field carbon not observed)

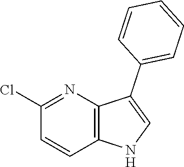 [SMILES: Clc1ccc2[nH]cc(-c3ccccc3)c2n1]

5-Chloro-3-phenyl-1H-pyrrolo[3,2-b]pyridine. To a solution of 2-chloro-5-hydrazinopyridine (71.3 mg, 0.5 mmol) in 4% w/w aqueous H₂SO₄ (5 mL) in a microwave tube was added (2,2-dimethoxyethyl)benzene (87.3 mg, 0.525 mmol). The reaction vessel was sealed with a silicon septum and stirred at 23° C. for 1 min then irradiated in microwave at 160° C. for 5 min. After the mixture was cooled to 23° C., it was slowly poured into 40% w/w KOH solution (5 mL). The mixture was extracted with EtOAc and the combined organic layer was dried over anhydrous MgSO₄, filtered and concentrated under reduced pressure. The resulting crude residue was purified by flash column chromatography (n-Hexane:EtOAc=3:2) to obtain the desired product (71.3 mg, 0.312 mmol, 62%) as a light yellow solid. ¹H NMR (400 MHz, CDCl₃) δ 8.96 (br s, 1H), 7.99 (d, J=7.2 Hz, 2H), 7.59 (s, 1H), 7.57 (d, J=8.8 Hz, 1H), 7.39 (t, J=7.6 Hz, 2H), 7.23 (dd, J=7.6, 7.2 Hz, 1H), 7.12 (d, J=8.9 Hz, 1H). The spectroscopic data match the literature data. [Ref: Eur. J. Org. Chem. 2013, 3328-3336.

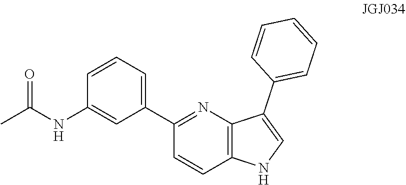 [SMILES: CC(=O)Nc1cccc(-c2ccc3[nH]cc(-c4ccccc4)c3n2)c1]

N-(3-(3-Phenyl-1H-pyrrolo[3,2-b]pyridin-5-yl)phenyl)acetamide, JGJ034. The reaction of 5-chloro-3-phenyl-1H-pyrrolo[3,2-b]pyridine (40 mg, 0.175 mmol), 3-aminophenylboronic acid (29.8 mg, 0.192 mmol), K₂CO₃ (36.3 mg, 0.262 mmol) and Pd(PPh₃)₄ (20.2 mg, 0.018 mmol) in 1,4-dioxane/water (5:1 v/v, 0.5 mL) afforded 3-(3-phenyl-1H-pyrrolo[3,2-b]pyridin-5-yl)aniline (18.8 mg, 0.066 mmol, 38%) as a white solid using the same procedure as described for JGJ002. Then the acetylation using the same procedure as described for JGJ004 gave the desired product JGJ034 (13.5 mg, 0.041 mmol, 63%) as an ivory solid. ¹H NMR (400 MHz, CD₃OD) δ 8.29 (s, 1H), 8.24 (d, J=7.2 Hz, 2H), 7.88 (s, 1H), 7.83 (d, J=7.6 Hz, 1H), 7.82 (d, J=8.8 Hz, 1H), 7.64 (d, J=8.4 Hz, 1H), 7.62 (d, J=7.6 Hz, 1H), 7.39-7.44 (m, 3H), 7.21 (dd, J=7.6, 7.2 Hz, 1H), 2.17 (s, 3H); ¹³C NMR (100 MHz, CD₃OD) δ 170.3, 150.1, 143.3, 141.1, 138.7, 134.5, 129.3, 128.5, 127.9, 126.3, 126.2, 125.1, 122.4, 119.3 (two peaks), 118.3, 115.7, 114.0, 22.4.

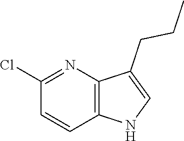

5-Chloro-3-propyl-1H-pyrrolo[3,2-b]pyridine. The reaction of 2-chloro-5-hydrazinopyridine (71.8 mg, 0.5 mmol) and valeraldehyde (45.1 mg, 0.524 mmol) in 4% w/w aq. H₂SO₄ (5 mL) afforded the desired product (56.7 mg, 0.291 mmol, 58%) as a pale yellow solid using the same procedure as described for 5-chloro-3-phenyl-1H-pyrrolo[3,2-b]pyridine. ¹H NMR (400 MHz, CDCl₃) δ 8.01 (br s, 1H), 7.61 (d, J=8.0 Hz, 1H), 7.26 (s, 1H), 7.08 (d, J=8.0 Hz, 1H), 2.77 (t, J=7.6 Hz, 2H), 1.73 (m, 2H), 0.94 (t, J=7.2 Hz, 3H); ¹³C NMR (100 MHz, CDCl₃) δ 145.0, 143.4, 127.8, 126.3, 120.9, 117.2, 116.6, 26.8, 23.0, 14.0.

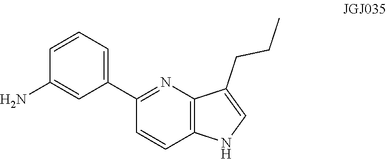 [SMILES: CCCc1c[nH]c2ccc(-c3cccc(N)c3)nc12]

3-(3-Propyl-1H-pyrrolo[3,2-b]pyridin-5-yl)aniline, JGJ035. The reaction of 5-chloro-3-propyl-1H-pyrrolo[3,2-b]pyridine (40 mg, 0.206 mmol), 3-aminophenylboronic acid (31 mg, 0.226 mmol), K₂CO₃ (42.6 mg, 0.308 mmol) and Pd(PPh₃)₄ (23.8 mg, 0.021 mmol) in 1,4-dioxane/water (5:1 v/v, 0.5 mL) afforded the desired product JGJ035 (42.5 mg, 0.169 mmol, 82%) as a white solid using the same procedure as described for JGJ002. ¹H NMR (400 MHz, CD₃OD) δ 7.72 (d, J=8.4 Hz, 1H), 7.44 (d, J=8.8 Hz, 1H), 7.34 (dd, J=2.0, 1.6 Hz, 1H), 7.30 (s, 1H), 7.24 (ddd, J=7.6, 1.6, 1.2 Hz, 1H), 7.19 (t, J=7.6 Hz, 1H), 6.76 (ddd, J=7.6, 2.0, 1.2 Hz, 1H), 2.85 (t, J=7.6 Hz, 2H), 1.79 (m, 2H), 1.02 (t, J=7.2 Hz, 3H); ¹³C NMR (100 MHz, CD₃OD) δ 152.4, 128.8, 146.2, 143.4, 130.2, 130.1, 127.6, 120.3, 118.8, 117.4, 116.3, 115.8, 27.1, 24.6, 14.5. (one low-field carbon not observed)

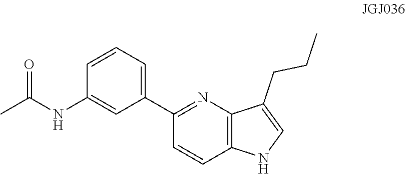 [SMILES: CCCc1c[nH]c2ccc(-c3cccc(NC(C)=O)c3)nc12]

N-(3-(3-Propyl-1H-pyrrolo[3,2-b]pyridin-5-yl)phenyl)acetamide, JGJ036. The reaction of 3-(3-propyl-1H-pyrrolo[3,2-b]pyridin-5-yl)aniline (JGJ035, 34.5 mg, 0.137 mmol), triethylamine (20.8 mg, 0.206 mmol) and acetyl chloride (16.2 mg, 0.206 mmol) in dichloromethane (3 mL) afforded the desired product JGJ036 (28.8 mg, 0.098 mmol, 72%) as an ivory solid using the same procedure as described for JGJ004. ¹H NMR (400 MHz, CD₃OD) δ 8.11 (dd, J=2.0, 1.6 Hz, 1H), 7.75 (d, J=8.4 Hz, 1H), 7.64-7.67 (m, 2H), 7.49 (d, J=8.8 Hz, 1H), 7.39 (t, J=8.0 Hz, 1H), 7.32 (s, 1H), 2.85 (t, J=7.2 Hz, 2H), 2.15 (s, 3H), 1.80 (m, 2H), 1.01 (t, J=7.2 Hz, 3H); ¹³C NMR (100 MHz, CD₃OD) δ 171.8, 151.4, 146.4, 143.1, 140.1, 130.3, 129.9, 127.9, 124.2, 120.6, 120.4, 120.2, 117.4, 115.7, 27.1, 24.5, 23.9, 14.5.

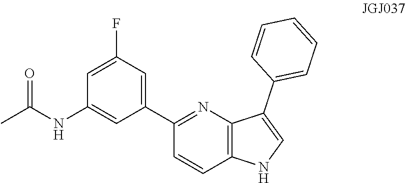 [SMILES: CC(=O)Nc1cc(F)cc(-c2ccc3[nH]cc(-c4ccccc4)c3n2)c1]

N-(3-Fluoro-5-(3-phenyl-1H-pyrrolo[3,2-b]pyridin-5-yl)phenyl)acetamide, JGJ037. The reaction of 5-chloro-3-phenyl-1H-pyrrolo[3,2-b]pyridine (19.4 mg, 0.085 mmol), 3-fluoro-5-aminophenylboronic acid (14.5 mg, 0.093 mmol), K₂CO₃ (17.6 mg, 0.127 mmol) and Pd(PPh₃)₄ (9.8 mg, 0.009 mmol) in 1,4-dioxane/water (5:1 v/v, 0.3 mL) afforded 3-fluoro-5-(3-phenyl-1H-pyrrolo[3,2-b]pyridin-5-yl)aniline (18.1 mg, 0.060 mmol, 70%) as an ivory solid using the same procedure as described for JGJ002. Then the acetylation using the same procedure as described for JGJ004 gave the desired product JGJ037 (13.8 mg, 0.040 mmol, 67%) as an ivory solid. ¹H NMR (400 MHz, CD₃OD) δ 8.25 (m, 2H), 7.98 (t, J=1.6 Hz, 1H), 7.88 (s, 1H), 7.79 (d, J=8.4 Hz, 1H), 7.61 (d, J=8.8 Hz, 1H), 7.58 (m, 2H), 7.43 (t, J=7.6 Hz, 2H), 7.21 (td, J=7.6, 1.2 Hz, 1H), 2.15 (s, 3H); ¹³C NMR (100 MHz, CD₃OD) δ 171.9, 164.6 (d, J=239.6 Hz), 150.2 (d, J=2.9 Hz), 145.1, 144.7 (d, J=8.9 Hz), 141.7 (d, J=11.5 Hz), 136.0, 130.9, 129.4, 127.8, 127.6, 126.6, 120.5, 117.3, 115.3, 114.7 (d, J=3.2 Hz), 109.8 (d, J=23.1 Hz), 107.3 (d, J=27.0 Hz), 24.0.

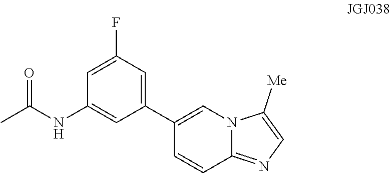 [SMILES: CC(=O)Nc1cc(F)cc(-c2ccc3ncc(C)n3c2)c1]

N-(3-Fluoro-5-(3-methylimidazo[1,2-a]pyridin-6-yl)phenyl)acetamide, JGJ038. The reaction of 6-bromo-3-methylimidazo[1,2-a]pyridine (23.4 mg, 0.111 mmol), 3-fluoro-5-aminophenyl-boronic acid (18.9 mg, 0.122 mmol), K₂CO₃ (23.0 mg, 0.166 mmol) and Pd(PPh₃)₄ (12.8 mg, 0.011 mmol) in 1,4-dioxane/water (5:1 v/v, 0.3 mL) afforded 3-fluoro-5-(3-methylimidazo[1,2-a]pyridin-6-yl)aniline (13.2 mg, 0.055 mmol, 49%) as a pale yellow solid using the same procedure as described for JGJ002. Then the acetylation using the same procedure as described for JGJ004 gave the desired product JGJ038 (8.3 mg, 0.029 mmol, 64%) as an ivory solid. ¹H NMR (400 MHz, CD₃OD) δ 8.41 (s, 1H), 7.56-7.63 (m, 3H), 7.52 (dt, J=10.8, 2.0 Hz, 1H), 7.40 (s, 1H), 7.23 (dt, J=9.6, 2.0 Hz, 1H), 2.57 (s, 3H), 2.17 (s, 3H);

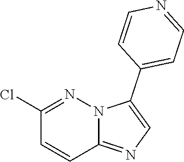 [SMILES: Clc1ccc2ncc(-c3ccncc3)n2n1]

6-Chloro-3-(pyridin-4-yl)imidazo[1,2-b]pyridazine. The reaction of 6-chloro-3-iodoimidazo[1,2-b]pyridazine (90.5 mg, 0.324 mmol), 4-pyridineboronic acid (43.8 mg, 0.356 mmol), K₂CO₃ (67.1 mg, 0.486 mmol) and Pd(PPh₃)₄ (37.4 mg, 0.032 mmol) in 1,4-dioxane/water (5:1 v/v, 0.7 mL) at 100° C. afforded the desired product (15.3 mg, 0.066 mmol, 20%) as a pale yellow solid using the same procedure as described for 6-chloro-3-phenylimidazo[1,2-b]pyridazine. ¹H NMR (400 MHz, CDCl₃) δ 8.72 (d, J=5.2 Hz, 2H), 8.23 (s, 1H), 7.98-8.02 (m, 3H), 7.18 (d, J=9.2 Hz, 1H); ¹³C NMR (100 MHz, CDCl₃) δ 150.2, 147.3, 139.8, 135.2, 134.9, 127.5, 126.1, 119.9, 119.4.

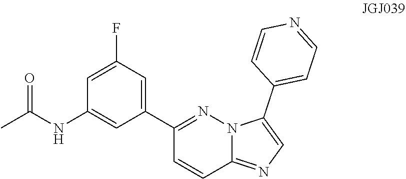 [SMILES: CC(=O)Nc1cc(F)cc(-c2ccc3ncc(-c4ccncc4)n3n2)c1]

N-(3-Fluoro-5-(3-(pyridin-4-yl)imidazo[1,2-b]pyridazin-6-yl)phenyl)acetamide, JGJ039. The reaction of 6-chloro-3-(pyridin-4-yl)imidazo[1,2-b]pyridazine (15.3 mg, 0.066 mmol), 3-fluoro-5-aminophenylboronic acid (11.3 mg, 0.073 mmol), K₂CO₃ (13.7 mg, 0.100 mmol) and Pd(PPh₃)₄ (7.7 mg, 0.007 mmol) in 1,4-dioxane/water (5:1 v/v, 0.3 mL) afforded 3-fluoro-5-(3-(pyridin-4-yl)imidazo[1,2-b]pyridazin-6-yl)aniline (10.7 mg, 0.035 mmol, 53%) as a light yellow solid using the same procedure as described for JGJ002. Then the acetylation using the same procedure as described for JGJ004 gave the desired product JGJ039 (3.8 mg, 0.011 mmol, 31%) as a pale yellow solid. ¹H NMR (400 MHz, CD₃OD) δ 8.69 (s, 2H), 8.46 (s, 1H), 8.36 (d, J=5.2 Hz, 2H), 8.20-8.23 (m, 2H), 7.88 (d, J=9.2 Hz, 1H), 7.64 (dt, J=10.8, 1.6 Hz, 1H), 7.55 (dt, 9.6, 1.6 Hz, 1H), 2.20 (s, 3H).

### Example 2: LIN28 is Significantly Overexpressed in Human and Murine AML and Drives MLL Leukemogenesis

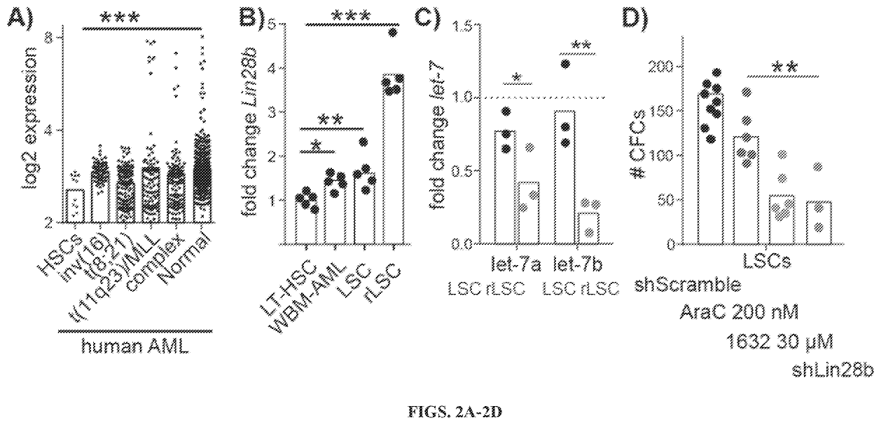

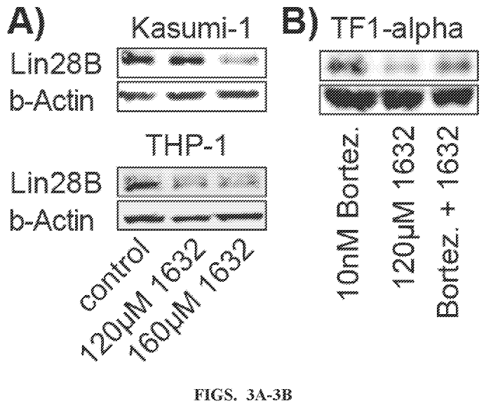

Analysis of databases of AML and healthy hematopoietic cells (HSCs, Blood Spot(55)) showed that Lin28b expression is significantly enriched in a variety of AML karyotypes, when compared to healthy HSCs (FIG. 2A). In addition, Lin28 was independently found to be a key driver in MLL-associated leukemias (56). In order to further characterize the role of Lin28/let-7 in regulating AML, LSC proliferation and therapy resistance in vivo, we used a doxycycline (DOX) inducible, transgenic mouse model for MLL-AF9-driven AML (iMLL-AF9 (57)). In this model, long-term HSC (LT-HSC, Lin⁻CD34⁻Sca-1⁻ c-Kit⁺ CD150⁺ CD48⁻) derived AML blasts closely mirror an LSC-like phenotype resulting in a particularly aggressive, Cytarabine (Ara-C) resistant AML (57). We transplanted congenic mice (B6.SJL, CD 45.1) with whole bone marrow (WBM) cells or fluorescent activated cell sorted (FACS) LT-HSCs from non-induced iMLL-AF9 mice and maintained the recipients on DOX. mRNA analysis of AML cells harvested at day (d)+35 showed that Lin28b expression was significantly increased in WBM- and LT-HSC derived AML cells (LSCs) when compared to healthy, non-DOX induced LT-HSCs (FIG. 2B). Moreover, relapsed AML cells arising from rLSCs after treatment with Ara-C (100 mg/kg) at d+60 were even further enriched in Lin28b expression (FIG. 2B). Additionally, levels of both, let-7a and let-7b miRNAs, negatively correlated with increased Lin28b in rLSCs (FIG. 2C) (10, 39). Our findings are consistent with papers reporting that increased Lin28 correlates with disease recurrence post chemotherapy in colon and liver cancer stem cells (22, 58).

### Example 3: Lin28 Inhibition Overcomes Therapy Resistance of Relapsed AML

As Lin28 is overexpressed human AML, LSCs and rLSCs, we sought to determine if genetic Lin28b or pharmacologic Lin28/let-7 inhibition via LN1632 can abrogate proliferation of LSCs and thus overcome their therapy resistance. We FACS isolated LT-HSCs and incubated 500 cells with DOX and concomitantly transduced them with either shLin28b or its corresponding control shScramble, or treated the cells with 200 nM Ara-C, 30 μM 1632 or control for 48 h. While Ara-C did not alter colony forming capacity cell numbers (CFCs), genetic silencing by (shLin28b) or pharmacologic inhibition of Lin28 by LN1632 treatment significantly abrogated CFCs of LSCs (FIG. 2D).

### Example 4: Targeted LIN28/Let-7 Inhibition Decreases Tumor Burden in AML In Vivo

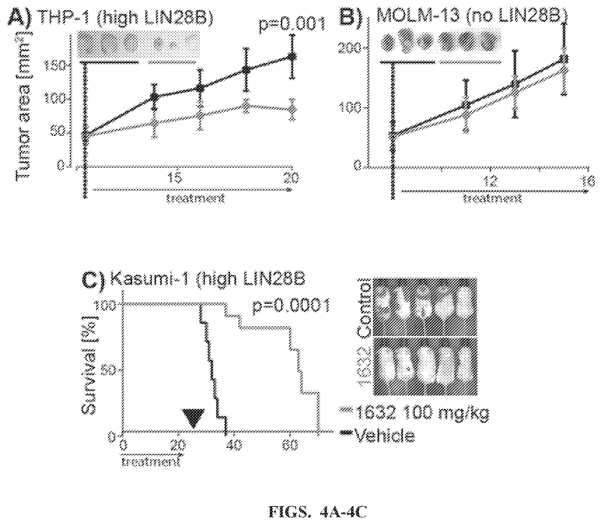

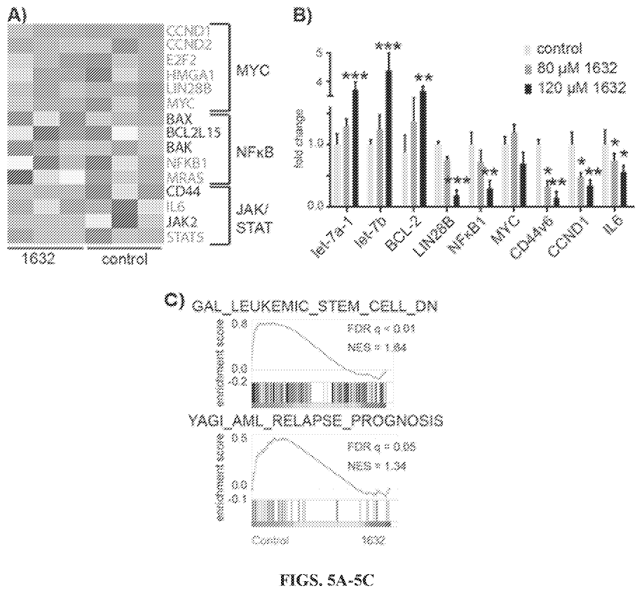

Given that genetic Lin28b inhibition and treatment with LN1632 abrogates CFCs of LSCs, we sought to explore the effect of LN1632 in human AML. By Western blotting, we confirmed that the Lin28 inhibitor LN1632 dose-dependently decreases LIN28B protein levels in AML with t(8;21) (Kasumi-1) and MLL-rearrangement (THP-1) (FIG. 3A). Of note, Bortezomib, a proteasome inhibitor, was able to inhibit decrease of LIN28B protein levels in TF1-alpha cells post treatment with 1632 (FIG. 3B) suggesting that 1632 may directly target LIN28B leading to its proteasomal degradation. As such, we investigated the effect of targeted Lin28/let-7 inhibition in AML in vivo. We established an intermitted dosing of 100 mg/kg every other day for 21 d as non-toxic, well tolerated in healthy C57BL/6 mice as they showed normal weight gain, complete blood counts (CBCs) and behavior. Thus, we subcutaneously (subQ) implanted THP-1 (high LIN28B) cells or MOLM-13 (no LIN28B) into NSG mice and 12 d later (tumor size=40 mm²) started administration of 1632 IP at 100 mg/kg every other day. Our results showed significantly reduced tumor growth in THP-1 but not MOLM-13 Xenografts (FIG. 4A-B). We further assessed the effect of 1632 in a systemic Kasumi-1 cell line Xenograft (LSC like CD34+CD38, high LIN28B, AML t(8;21)). IP injection with 1632 at 100 mg/kg every other day for 21 d significantly prolonged animal survival (FIG. 4B). Bioluminescence imaging (BLI) confirmed decreased tumor burden in 1632 treated mice compared to vehicle (FIG. 4C, picture).

### Example 5: Targeted Inhibition of LIN28 Downregulates NF-κB and BCL-2 in Primary AML

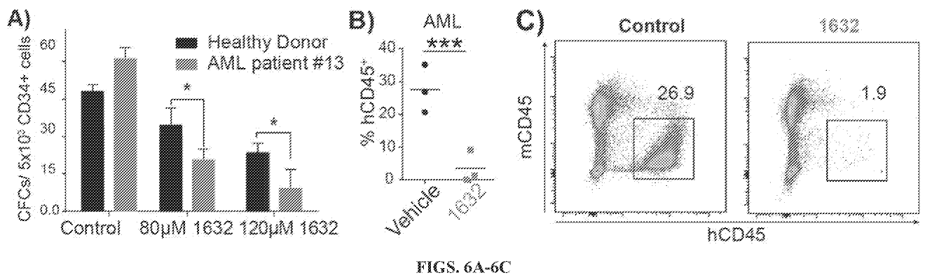

To measure the complete degree to which LN1632 regulates gene expression, we carried out RNA sequencing (RNAseq) in LSC-like Kasumi-1 cells. As depicted in the heatmap in FIG. 5A, we found significant downregulation of a panoply of direct let-7 target genes (44) including CCND1/2, E2F2, HMGA1, LIN28B, MYC, NFKB1, MRAS, IL6 and STAT5 (in green, FIG. 5A). Importantly, we confirmed this gene expression pattern in primary AML cells from three relapsed patients (validated for LIN28B overexpression compared to healthy WBM). 72 h post treatment, we found dose-dependent, significant upregulation of mature let-7a/b and downregulation of multiple let-7 target genes (FIG. 5B), including NFκB1. This is important because NFκB1, regulated by let-7 through IL6 (23) is, together with other BCL-2 family members (BAX, BCL2L15 and BMF, FIG. 5A), a well characterized gene associated with unique properties of LSC survival and AML relapse (45, 59, 60). In line with this, gene set enrichment analysis revealed a global change of gene expression signatures previously shown to discriminate LSCs from non-self-renewing leukemia cell populations (61) and poor pediatric AML relapse prognosis (62) (FIG. 5C). We next explored the effect of LN1632 on primary AML cells. CFC assays in FIG. 6A show that treatment with LN1632 affects colony formation of CD34⁺ AML pt. #13 cells significantly more than healthy CD34⁺BM cells. Moreover, ex-vivo treatment of AML pt #13 cells with 1632 or control inhibited AML repopulation capability in vivo (FIG. 6B-C). Thus, our results suggest that effects of LN1632 are greater on LSCs than HSCs.

### Example 6: Lin28/Let-7 Inhibitory Activity of Exemplary Compounds

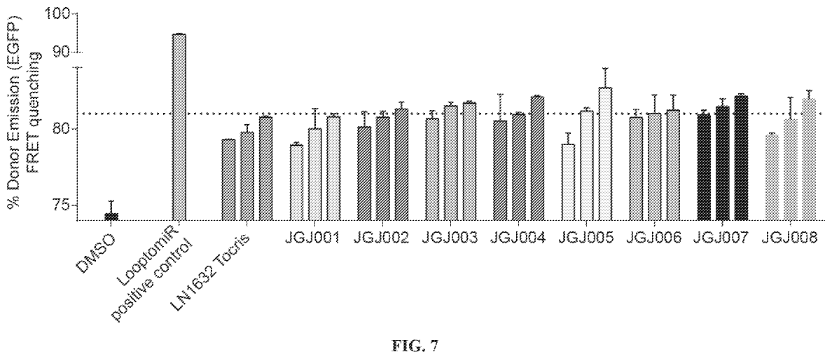

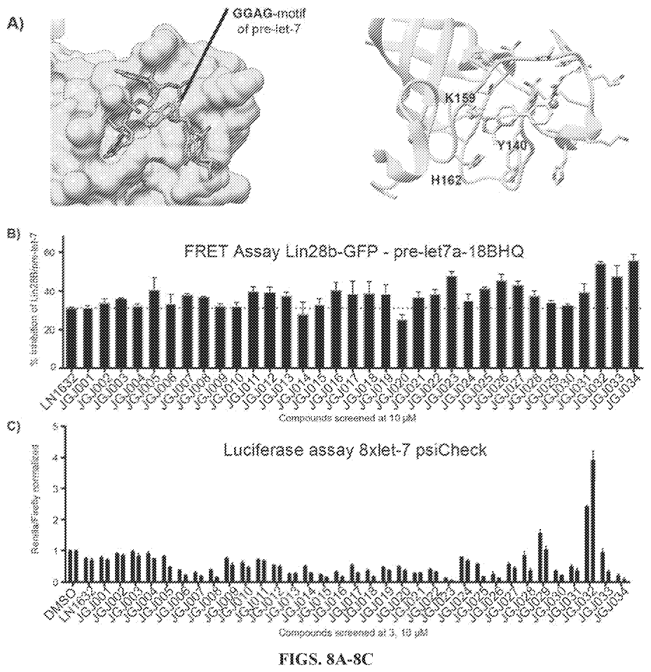

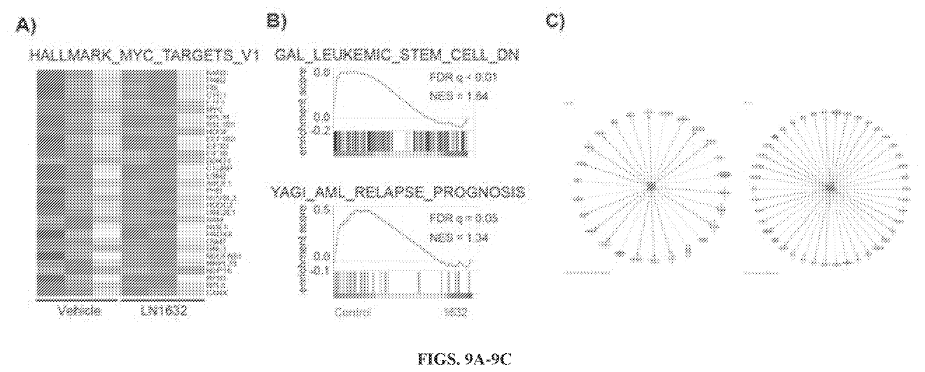

In order to improve binding and inhibitory capacity of a compound to LIN28b, we predicted the binding mode of LN1632 to LIN28B. Close-ups of crystal structure of LIN28 protein revealed possible binding mode of LN1632 to the GGAG-RNA sequence binding pocket of the CCHC domains of LIN28 (not shown). With this model in hand, we synthesized novel compounds with improved binding capacity to Lin28b (JGJ002-JGJ008, FIG. 8). Compounds were screened using a previously described FRET-assay with an EGFP-tagged LIN28B as donor and a BHQ-1 quencher labelled pre-let-7a-2 (pre-let-7a-2-BHQ1) as acceptor (51). Briefly, recombinant LIN28B-EGFP was harvested from stably transduced HEK cells and diluted with binding buffer (300 mM NaCl, 25 mM HEPES pH 7.2, 10 μM ZnCl2, 1% Odyssey Blocking Buffer, 0.05% Tween 20, 0.5 mM TCEP) in order to adjust ideal FRET-quenching signal intensity. Protein lysate and compounds (JGJ001-JGJ008) were pre-incubated in doses ranging from 1.25 uM-20 uM for 20 min in 100 uL diluted protein lysate. Subsequently pre-let-7a-2-BHQ1 was added to the mixture (at 6.25 nM) and EGFP-LIN28B donor emission was measured using a Tecan Spark Plate Reader (20 nM band with, excitation at 488 nM, emission read-out at 545 nM, 30 flashes/s). Results show that particularly compounds JGJ005, JGJ007 and JGJ008 inhibit FRET-signal intensity to a greater degree than the original hit compound LN1632. From these results, we conclude that JGJ005, JGJ007 and JGJ008 show greater inhibition of the LIN28B/pre-let-7a2 binding than the original compound LN1632 (FIG. 7), and thus are expected to inhibit LIN28 from binding to pre-let-7 microRNAs, thereby preventing their degradation. Increased endogenous let-7 miRNA levels can target a panoply of LCS and cancer stem cell hallmark genes thus inhibiting tumor growth.

### Example 7: Evaluation of LN1632 Activity In Vitro and In Vivo

Using a targeted high-throughput fluorescence resonance electron transfer (FRET) screen, triazolopyridazines was identified as a class of small molecules which block the interaction of RBP LIN28 and pre-let-7 miRNA (51). To study how LN1632 interacts with LIN28 protein, in silico molecular docking studies were performed using a crystal structure of the LIN28B pre-let-7a complex (PDB ID: 5UDZ)(28). Based on LN1632's ability to compete for the LIN28B-pre-let-7 complex in the FRET assay, it was hypothesized that the binding site is likely to be shared with the ZKD RNA binding motif of LIN28. The docking results showed that LN1632 binds to the pocket that is originally occupied by the GGAG motif of pre-let-7a. The results also demonstrated that the amide group of LN1632's phenyl ring is positioned at a pocket near the binding site of the zinc ion through H-bonding interaction with LIN28B. (FIG. 8A).

To test the structure-activity relationship, 39 LN1632-related analogs (JGJ001-39) were synthesized and their potency and specificity to inhibit LIN28B-RNA binding activity and upregulate mature let-7 miRNA levels was measured. By performing the previously published FRET assay, it was observed that JGJ023, JGJ026, JGJ032 and JGJ034 inhibit LIN28's RNA-binding capacity significantly more than compound LN1632 (FIG. 8B). Additionally, in HepG2 cells, JGJ023, JGJ026 and JGJ034 upregulated mature let-7 miRNAs at significantly lower doses than LN1632, as measured by a dual luciferase reporter assay (FIG. 8C). The dual luciferase reporter assay was performed as previously described (89).

To determine the degree to which LN1632 regulates gene expression, RNA sequencing was performed in human Kasumi-1 AML cells. The data in FIGS. 9A-9B showed that treatment of cells with LN1632 significantly downregulated genes of the HALLMARK_MYC-TARGETS_V1 gene signature (70), leukemic stem cell and relapse prognosis signatures (61, 62). In addition, ingenuity pathway analysis predicted suppression of upstream signaling molecules IL6 and MYC (FIG. 9C).

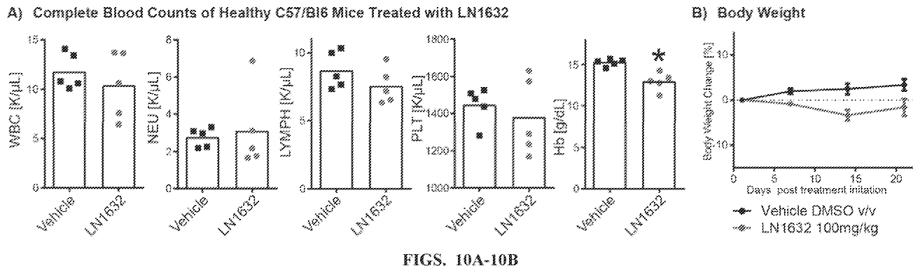

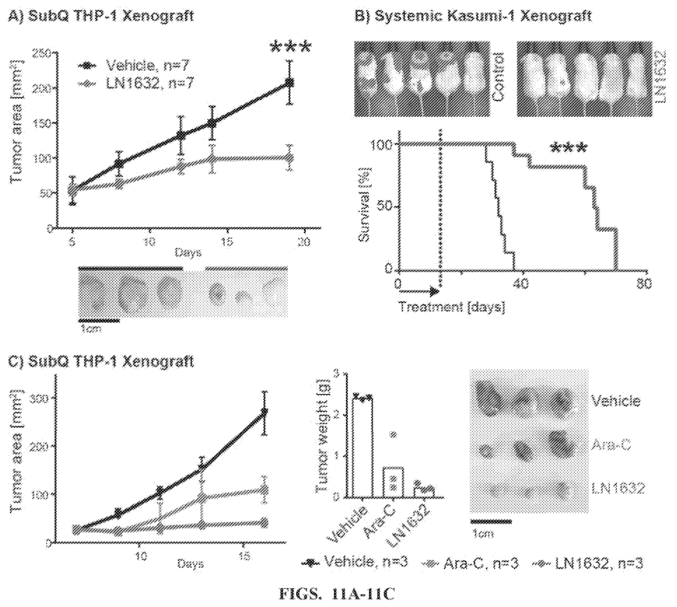

Next, the tumor suppressive effect of LN1632 in vivo was investigated. The maximum tolerated dose (MTD) was evaluated in healthy female C57Bl/6 mice. Dosing of 100 mg/kg daily for +12 d followed by an every-other-day dosing schedule for +9 d was well tolerated and mice showed a normal complete blood count (CBC) profile, without any leukopenia or thrombocytopenia, only mild anemia and normal weight gain (FIG. 10A-B).

Subsequently, the tumor suppressive effect of LN1632 in cancer in vivo was assessed. High LIN28B expressing THP-1 AML cells (2×10⁶ cells) were implanted in NSGS mice (cell suspension in matrigel, 3:1) and at d+12 or d+8 (tumor size=50 mm²) injection of LN1632 IP at 100 mg/kg daily was initiated. The results showed significantly reduced tumor growth 19 days post injection (FIG. 11A). These results are in line with a recent report showing that LN1632 selectively suppresses LIN28B-expressing Ewing Sarcoma (EwS) but not LIN28B depleted EwS (72) and LIN28B expressing TNBC cells (73). The effect of LN1632 in a systemic Kasumi-1 xenograft was also assessed. IP injection with LN1632 at 100 mg/kg every other day for 21 d significantly prolonged animal survival (FIG. 11B). Bioluminescence imaging (BLI) confirmed decreased tumor burden in LN1632 treated mice compared to vehicle (FIG. 11B, picture). The effects of LN1632 to cytarabine chemotherapy (Ara-C) was also compared, as previously described (74). THP-1 AML cells were subcutaneously implanted (1.5×10⁶ cells, high LIN28B) in NSGS mice. Daily IP injections with 100 mg/kg LN1632, 60 mg/kg cytarabine chemotherapy (Ara-C), or vehicle was started at +d3 post AML cell implantation and continued until the vehicle group reached maximal allowed tumor size (250 mm²). LN1632 treated mice showed increased inhibition of AML tumor proliferation compared to Ara-C or vehicle treated mice (FIG. 11C).

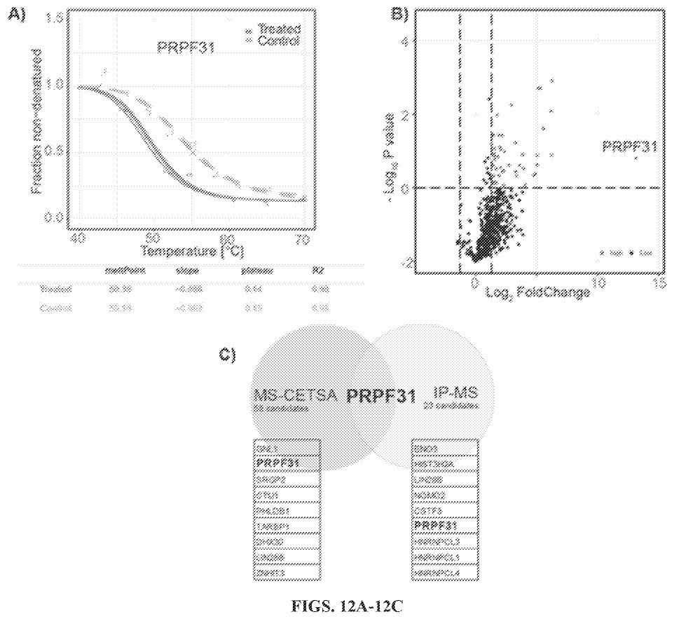

As LN1632 showed significant antiproliferative effects in cancer in vivo models, additional functional interaction partners of LN1632 were evaluated. Mass spectrometry cellular thermal shift assay (MS-CETSA, FIG. 12A) as described in (75), and immunoprecipitation using biotinylated LN1632 (FIG. 12B) were performed. These experiments demonstrated that LN1632 interacts with additional RNA-binding proteins, in particular pre-mRNA processing factor 31 (FIG. 12C, PRPF31). PRPF31 is a component of the spliceosome complex, and is significantly overexpressed in embryonic stem cells (76) and downregulated during differentiation (77). PRPF31 is recruited to introns where its highly conserved Nop-domain coordinates the U4 snRNA—15.5K protein interaction. Subsequently, PRPF31 stabilizes the U4/U6.U5 tri-snRNP by concomitantly interacting with PRPF6 and induces the transition of the spliceosomal complex to the activated state (78).

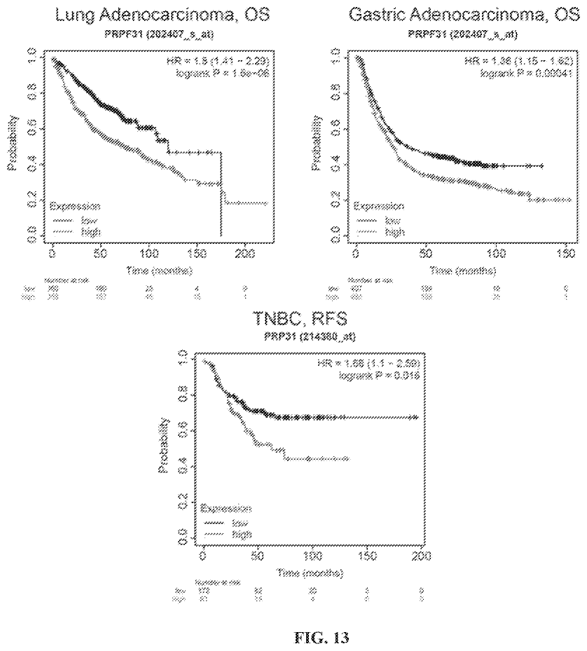

As shown in FIG. 13, PRPF31 overexpression correlates with poor prognosis in various tumors, including lung- and gastric adenocarcinomas as well as triple negative breast cancer (TNBC) (FIG. 13). Dysregulation of components of the U4/U6.U5 tri-snRNP complex have been shown to drive tumorigenesis in colorectal cancer (79), TNBC (80-82), hepatocellular carcinoma (83) and lung cancer. Dysfunctional RNA splicing and overexpression of splicing factors is an essential mechanism for tumor cell survival and intersects with many hallmarks of cancer (84-86). Emerging studies show that, in some embodiments, components of the spliceosome are essential for the oncoprotein MYC to drive cancer progression. Without wishing to be bound by any particular theory, since MYC is the most frequently amplified oncogene in human cancers and plays a crucial role in malignant transformation, in some embodiments, therapies that exploit the spliceosome, and target PRPF31 and the U4/U6 spliceosome complex in particular, would be very attractive.

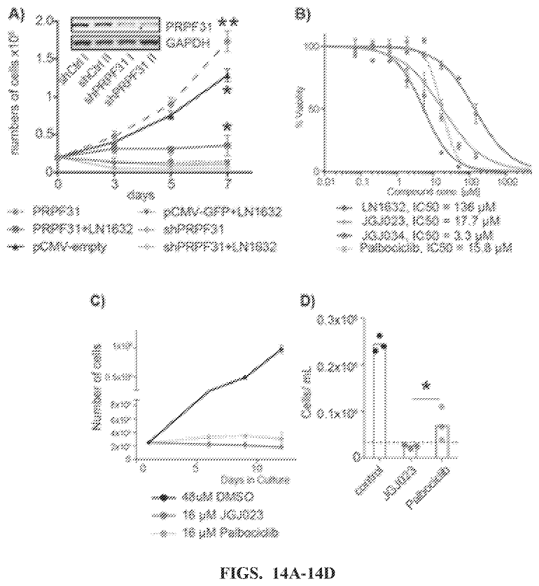

MDA-MB-231 TNBC cells were used to evaluate whether LN1632 targets PRPF31. Overexpression of PRPF31 increased cell proliferation while genetic silencing of PRPF31 significantly reduced cell number as assessed over 7 days (FIG. 14A). Importantly, PRPF31 overexpression (pLenti-C-mGFP-P2A-Puro-PRPF31, Origene) rescued anti-proliferative effects of LN1632, indicating that LN1632 targets PRPF31. Additionally, genetic silencing of PRPF31 via short-hairpin mediated RNA (shRNA, ThermoFisher Scientific, TRCN0000001180) abrogated pro-apoptotic effects of LN1632. In summary, these results indicate that LN1632 targets PRPF31 (FIG. 14A).

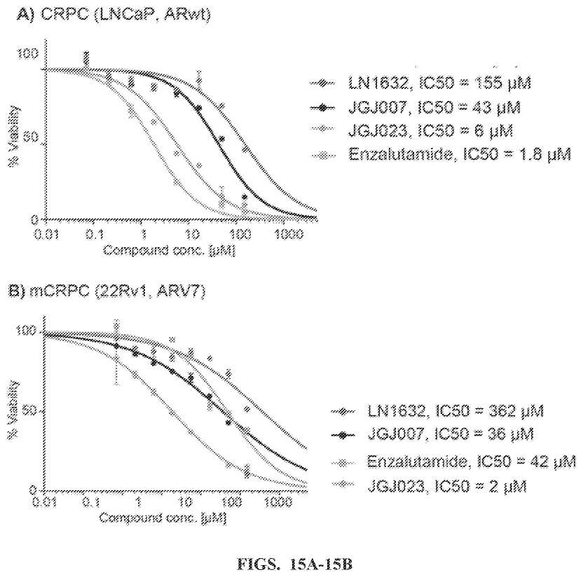

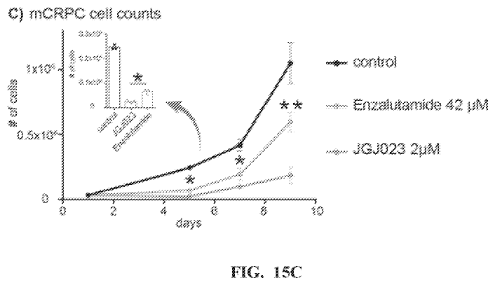

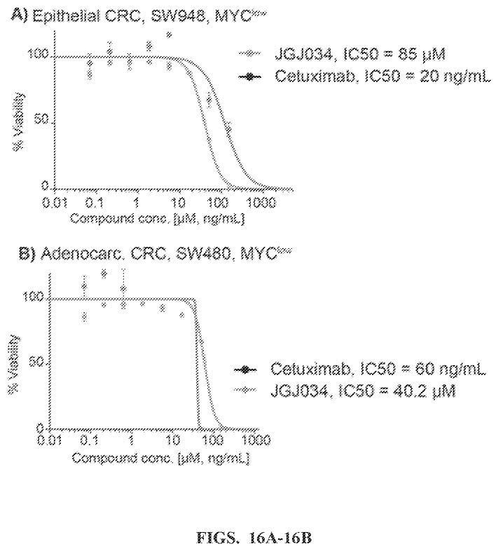

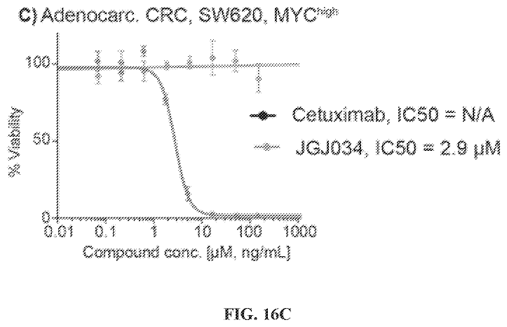

To test whether LN1632 and novel analogs thereof affect cancer cell growth, cell viability and cell counting assays in TNBC (FIG. 14B-D), castration resistant prostate cancer cells (CRPC, FIG. 15A-C) and colorectal cancer cells (CRC, FIG. 15A-C) were performed. The data showed that LN1632 and novel analogs JGJ034 and JGJ037 reduced proliferation and induced apoptosis preferentially in MYC-driven cancers, including TNBC, CRPC, lung and colorectal adenocarcinoma cells (Table 1).

To measure cell viability, cell titer glow (CTG, Promega CellTiter-Glo 2.0 Assay) and MTT assays (SigmaAldrich, Cell Proliferation Kit I) were performed. Briefly, cells were serum starved overnight prior to seeding in 96-well plates. Following 24 h incubation, cells were treated with increasing concentrations of JGJ compounds for 96 hr. At assay read-out, CellTiter-Glo reagent was added and luminescence was measured after 10-minutes incubation at room temperature. For the MTT assay, MTT labeling reagent was added and incubated for 4 h. Subsequently, medium was removed and 50 μL DMSO was added to solubilize the crystals, and absorbance was measured at 570 nm. Cell viability was calculated as (Sample-Background)/(Control-Background). Current standard of care drugs, Enzalutamide, Palbociclib and Cetuximab were used as comparative controls.

TABLE 1

| Cell Line | Chemical Compound | IC₅₀ | | Assay Protocol |
|---|---|---|---|---|
| Prostate | | | | |
| 22RV1 | LN1632 | 236 | μM | MTT |
| | JGJ007 | 97 | μM | MTT |
| | JGJ015 | 209 | μM | MTT |
| | JGJ023 | 2 μM, 8 μM | | MTT, CTG |
| | JGJ034 | 1 μM, 2 μM | | MTT, CTG |
| | JGJ037 | 2 μM, 3 μM | | MTT, CTG |
| | Enzalutamide | 53 μM, 88 μM | | MTT, CTG |
| LNCaP | LN1632 | 110 | μM | MTT |
| | JGJ007 | 54 | μM | MTT |
| | JGJ015 | 83 | μM | MTT |
| | JGJ023 | 6 | μM | MTT |
| | JGJ034 | 3 | μM | MTT |
| | Ezalutamide | 5 | μM | MTT |
| DU145 | LN1632 | NA | | MTT |
| | JGJ007 | 5 | μM | MTT |
| | JGJ015 | 30 | μM | MTT |
| | JGJ023 | 3 | μM | MTT |
| Lung | | | | |
| H1975 | LN1632 | 91 | μM | MTT |
| | JGJ007 | 31 | μM | MTT |
| | JGJ015 | 51 | μM | MTT |
| | JGJ023 | 8 | μM | MTT |
| | Osimertinib | 21 | μM | MTT |
| | MRTX849 | 4 | μM | MTT |
| H1568 | LN1632 | 128 | μM | MTT |
| | JGJ007 | 47 | μM | MTT |
| | JGJ023 | 12 | μM | MTT |
| A549 | LN1632 | 151 | μM | MTT |
| | JGJ007 | 97 | μM | MTT |
| | JGJ015 | 93 | μM | MTT |
| | JGJ023 | 20 | μM | MTT |
| | Osimertnib | 11 | μM | MTT |
| | MRTX849 | 4 | μM | MTT |
| H23 | LN1632 | 167 | μM | MTT |
| | JGJ007 | 95 | μM | MTT |
| | JGJ015 | 62 | μM | MTT |
| | JGJ023 | 94 | μM | MTT |
| H460 | LN1632 | 172 | μM | MTT |
| | JGJ007 | 512 | μM | MTT |
| | JGJ015 | 594 | μM | MTT |
| | JGJ023 | 124 | μM | MTT |
| Breast | | | | |
| BT-549 | LN1632 | 151 | μM | CTG |
| | JGJ007 | 75 | μM | CTG |
| | JGJ015 | 48 | μM | CTG |
| | JGJ023 | 7 | μM | MTT |
| | Palbociclib | 72 | μM | MTT |
| MDA- | LN1632 | 136 | μM | MTT |
| MB231 | | | | |
| | JGJ007 | 39 μM, 43 μM | | MTT, CTG |
| | JGJ015 | 145 μM, 75 μM | | MTT, CTG |
| | JGJ023 | 61 μM, 17 μM | | MTT, CTG |
| | JGJ034 | 3 | μM | CTG |
| | JGJ037 | 11 | μM | CTG |
| | Palbociclib | 13 | μM | CTG |
| MCF7 | LN1632 | 144 | μM | MTT |
| | JGJ007 | 45 μM, 115 μM | | MTT, CTG |
| | JGJ015 | 59 | μM | MTT |
| | JGJ023 | 38 | μM | MTT |
| | JGJ037 | 15 | μM | CTG |
| | Palbociclib | 5 μM, 32 μM | | MTT, CTG |
| Colon | | | | |
| HCT-116 | LN1632 | 158 | μM | CTG |
| | JGJ007 | 148 | μM | CTG |
| | JGJ015 | 148 | μM | CTG |
| | JGJ023 | 215 | μM | CTG |
| | JGJ034 | 43 | μM | CTG |
| | JGJ037 | 76 | μM | CTG |
| SW620 | LN1632 | 161 μM, 191 μM | | MTT, CTG |
| | JGJ007 | 64 | μM | MTT |
| | JGJ015 | 62 | μM | MTT |
| | JGJ023 | 27 | μM | CTG |
| | JGJ034 | 2 μM, 3 μM | | MTT, CTG |
| | JGJ037 | 8.5 μM, 15 μM | | MTT, CTG |
| | Cetuximab | NA | | CTG |
| SW480 | LN1632 | 155 μM, 168 μM | | MTT, CTG |
| | JGJ007 | 64 μM, 68 μM | | MTT, CTG |
| | JGJ015 | 53 μM, 110 μM | | MTT, CTG |
| | JGJ034 | 33 μM, 41 μM | | MTT, CTG |
| | JGJ037 | 26 μM, 39 μM | | MTT, CTG |
| | Cetuximab | 40 | μg/mL | CTG |
| SW948 | LN1632 | NA | | CTG |
| | JGJ023 | 51 | μM | CTG |
| | JGJ034 | 85 | μM | CTG |
| Pancreatic | | | | |
| ASPC-1 | LN1632 | 111 | μM | CTG |
| | JGJ007 | 188 | μM | CTG |
| | JGJ015 | 157 | μM | CTG |
| | JGJ037 | 31 | μM | CTG |
| CAPAN-1 | LN1632 | 82 | μM | CTG |
| | JGJ007 | 91 | μM | CTG |
| | JGJ015 | 137 | μM | CTG |
| | JGJ037 | 22 | μM | CTG |

The in vitro ADME characteristics of selected analogs are summarized in Tables 2 and 3.

TABLE 2

| | Caco2 permeability | | | Microsomal stability | | |
|---|---|---|---|---|---|---|
| | Conc. | Pₐₚₚ | Mean A-B | Conc. | | |
| Cpds No | (uM) | (10⁻⁶ cm/s) | % Recovery | (uM) | CD-1 Mouse | Human |
| LN1632 | 100 | A-B: 20.9 | 89.4 | 100 | T½ = 202 min | ND |
| | | B-A: 22.7 | | | % Remaining at | |
| | | efflux raio: | | | T30 = 90.2 | |
| | | 1.08 | | | CLint(mL/min/ | |
| | | | | | kg) = 27 | |
| JGJ007 | 30 | A-B: 36.2 | 87 | 30 | T½ = 21.2 min | T½ = 65.6 min |
| | | B-A: 30.0 | | | % Remaining at | % Remaining at |
| | | efflux | | | T30 = 37.6 | T30 = 72.8 |
| | | ratio: 0.829 | | | CLint(mL/min/ | CLint(mL/min/ |
| | | | | | kg) = 257 | kg) = 19 |
| JGJ015 | 30 | A-B: 24.1 | 67.3 | 30 | T½ = 5.52 min | T½ = 7.76 min |
| | | B-A: 21.1 | | | % Remaining at | % Remaining at |
| | | efflux | | | T30 = 2.3 | T30 = 6.9 |
| | | ratio: 0.874 | | | CLint(mL/min/ | CLint(mL/min/ |
| | | | | | kg) = 988 | kg) = 161 |
| JGJ026 | 5 | A-B: 27.2 | 84 | 5 | T½ = 11.8 min | T½ = 36.2 min |
| | | B-A: 25.01 | | | % Remaining at | % Remaining at |
| | | efflux | | | T30 = 17 | T30 = 54.7 |
| | | ratio: 0.6 | | | CLint(uL/min/ | CLint(uL/min/ |
| | | | | | mg) = 117.8 | mg) = 38.24 |
| JGJ037 | 5 | A-B: 0.44 | 47 | 5 | T½ = 93.0 min | T½ = >120 min |
| | | B-A: 1.69 | | | % Remaining at | % Remaining at |
| | | efflux | | | T30 = 78.6 | T30 = 91.2 |
| | | ratio: 3.9 | | | CLint(uL/min/ | CLint(uL/min/ |
| | | | | | mg) = 14.906 | mg) = 11.55 |

TABLE 3

| | CYP inhibition | | | | | | | | |
|---|---|---|---|---|---|---|---|---|---|
| Cpds | Conc. | | | | | | | | |
| No | (uM) | 1A2 | 2B6 | 2C8 | 2C9 | 2C19 | 2D6 | 3A4 | 3A4 |
| JGJ007 | 30 | 97.3 | 45.39 | 19.11 | 24.65 | 37.08 | 54.08 | 84.67 | 77.64 |
| JGJ015 | 30 | 96.4 | 67.27 | 17.82 | 14.27 | 49.8 | 19.38 | 38.08 | 69.37 |
| JGJ026 | 10 | 96.2 | 30.9 | 61.1 | 48.8 | 35 | 67.8 | 75.4 | 72.5 |
| JGJ037 | 10 | 88.9 | 69.6 | 90.5 | 86.6 | 98.3 | 93.9 | 99.7 | 98.2 |

### Example 8: Synthesis of LN1632 Analogues (JGJ Compounds)

General Experimental Methods

All reactions were carried out under an argon atmosphere unless otherwise specified. Tetrahydrofuran (THF) was distilled from benzoquinone ketyl radical under an argon atmosphere. Dichloromethane and triethylamine were distilled from calcium hydride under an argon atmosphere. All other solvents and reagents were purified according to literature procedures or purchased from Sigma-Aldrich, Acros, Oakwood and Fisher Scientific Co. ¹H NMR spectra were recorded at 400 or 500 MHz and are reported relative to deuterated solvent signals. Data for ¹H NMR spectra are reported as follows: chemical shift (δ ppm), multiplicity, coupling constant (Hz), and integration. Splitting patterns are designated as follows: s, singlet; d, doublet; t, triplet; q, quartet; m, multiplet; and br, broad. ¹³C NMR spectra were recorded at 100 or 125 MHz. Data for ¹³C NMR spectra are reported in terms of chemical shift. The chemical shifts are reported in parts per million (ppm, δ). Thin-layer chromatography (TLC) was carried out using precoated silica gel sheets. Visual detection was performed using potassium permanganate or ceric ammonium nitrate stains. Flash chromatography was performed using SilicaFlash P60 (60 A, 40-63 μm) silica gel with compressed air.

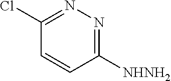

### 3-Chloro-6-hydrazineylpyridazine

To a solution of 3,6-dichloropyridazine (400 mg, 2.686 mmol) in EtOH (8 mL) was added hydrazine monohydrate (148 mg, 2.954 mmol) and the mixture was stirred at 100° C. for 3 h. After the mixture was cooled to 23° C., the resulting solid was collected and washed with Et₂O. The mother liquor was concentrated and the precipitate was washed with Et₂O. The combined solid was washed with dichloromethane to obtain the desired product (pale yellow, 320.2 mg, 2.216 mmol, 82%) and used for the next step without further purification. ¹H NMR (400 MHz, DMSO-d₆) δ 8.24 (br s, 1H), 7.41 (d, J=9.6 Hz, 1H), 7.09 (d, J=9.2 Hz, 1H), 4.37 (br s, 2H); ¹³C NMR (100 MHz, DMSO-d₆) δ 161.8, 145.4, 128.7, 116.1. Spectroscopic data match the literature data. [Ref: Heterocycles, 2009, 78 (4) 961-975]

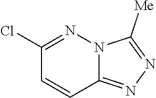 [SMILES: Cc1nnc2ccc(Cl)nn12]

### 6-Chloro-3-methyl-[1,2,4]triazolo[4,3-b]pyridazine

A mixture of 3-chloro-6-hydrazineylpyridazine (300 mg, 2.075 mmol) in AcOH (1.5 mL) was heated at 100° C. for 2 h. After the reaction mixture was cooled to 23° C., it was diluted with water and extracted with EtOAc. The combined organic layer was washed with sat. NaHCO₃ solution and brine, dried over anhydrous MgSO₄, filtered and concentrated under reduced pressure. The resulting crude off-white solid (238.5 mg, 68%) was used for the next step without further purification. ¹H NMR (400 MHz, CDCl₃) δ 8.04 (d, J=9.6 Hz, 1H), 7.09 (d, J=9.6 Hz, 1H), 2.81 (s, 3H).

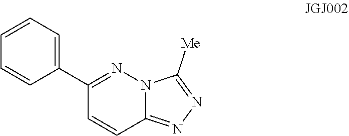 [SMILES: Cc1nnc2ccc(-c3ccccc3)nn12]

3-Methyl-6-phenyl-[1,2,4]triazolo[4,3-b]pyridazine, JGJ002. A mixture of 6-chloro-3-methyl-[1,2,4]triazolo[4,3-b]pyridazine (20 mg, 0.119 mmol), phenylboronic acid (14.5 mg, 0.119 mmol), K₂CO₃ (24.6 mg, 0.178 mmol) and Pd(PPh₃)₄ (13.6 mg, 0.012 mmol) in 1,4-dioxane (0.3 mL) and water (30 DL) was heated at 110° C. for 18 h. After the reaction mixture was cooled to 23° C., it was diluted with water and EtOAc. The organic layer was isolated and the aqueous layer was extracted with EtOAc. The combined organic layer was washed with brine, dried over anhydrous MgSO₄, filtered and concentrated under reduced pressure. The resulting crude residue was purified by flash column chromatography (dichloromethane:MeOH=10:1) to obtain the desired product JGJ002 (20.4 mg, 0.098 mmol, 82%) as an ivory solid. ¹H NMR (400 MHz, CDCl₃) δ. 8.13 (d, J=9.2 Hz, 1H), 7.98-8.01 (m, 2H), 7.54-7.56 (4H, m), 2.88 (s, 3H)¹³C NMR (100 MHz, CDCl₃) δ 153.4, 147.5, 143.4, 134.4, 130.9, 129.2, 127.2, 124.9, 118.8, 9.8.

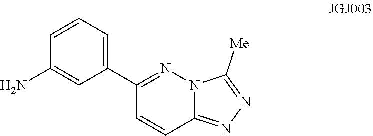 [SMILES: Cc1nnc2ccc(-c3cccc(N)c3)nn12]

3-(3-Methyl-[1,2,4]triazolo[4,3-b]pyridazin-6-yl)aniline, JGJ003. The reaction of 6-chloro-3-methyl-[1,2,4]triazolo[4,3-b]pyridazine (30 mg, 0.178 mmol), 3-nitrophenylboronic acid (35.6 mg, 0.214 mmol), K₂CO₃ (36.9 mg, 0.267 mmol) and Pd(PPh₃)₄ (20.6 mg, 0.018 mmol) in 1,4-dioxane (0.3 mL) and water (30 μL) afforded 3-methyl-6-(3-nitrophenyl)-[1,2,4]triazolo[4,3-b]pyridazine (19.7 mg, 0.077 mmol, 43%) using same procedure as described above. ¹H NMR (400 MHz, CDCl₃) δ. 8.86 (t, J=2.0 Hz, 1H), 8.39 (m, 2H), 8.24 (d, J=9.6 Hz, 1H), 7.71 (t, J=8.0 Hz, 1H), 7.62 (d, J=9.6 Hz, 1H), 2.91 (s, 3H)¹³C NMR (100 MHz, CDCl₃) δ 151.1, 148.8, 147.7, 143.2, 136.1, 132.8, 130.4, 125.8, 125.4, 122.2, 118.0, 9.9. Then a mixture of the nitro compound (19.4 mg, 0.076 mmol) and SnCl₂ (72.1 mg, 0.380 mmol) in EtOH (0.2 mL) was heated at reflux for 1 h. After the mixture was cooled to 23° C., it was filtered through Celite pad and washed with EtOAc. To the mixture was added sat. NaHCO₃ solution and it was extracted with EtOAc. The combined organic layer was washed with brine, dried over anhydrous MgSO₄, filtered and concentrated under reduced pressure. The resulting crude residue was purified by flash column chromatography (dichloromethane:MeOH=10:1) to obtain the desired product JGJ003 (10 mg, 0.044 mmol, 63%) as a pale yellow solid. ¹H NMR (400 MHz, CDCl₃) δ 8.10 (d, J=10.0 Hz, 1H), 7.51 (d, J=10.0 Hz, 1H), 7.26-7.32 (m, 3H), 6.83-6.86 (m, 1H), 2.86 (s, 3H); ¹³C NMR (100 MHz, CDCl₃) δ 153.5, 147.3 (two peaks overlapped), 143.4, 135.2, 130.0, 124.4, 119.1, 117.4, 117.2, 113.1, 9.7.

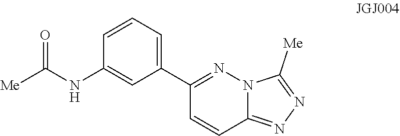 [SMILES: CC(=O)Nc1cccc(-c2ccc3nnc(C)n3n2)c1]

N-(3-(3-Methyl-[1,2,4]triazolo[4,3-b]pyridazin-6-yl)phenyl)acetamide, JGJ004. To a solution of 3-(3-methyl-[1,2,4]triazolo[4,3-b]pyridazin-6-yl)aniline (JGJ003, 20 mg, 0.088 mmol) in dichloro-methane (0.5 mL) was added trimethylamine (10.8 mg, 0.106 mmol) and acetyl chloride (7.6 mg, 0.099 mmol). The mixture was stirred at 23° C. for 6 h. To this mixture was added water and it was extracted with dichloromethane. The combined organic layer was washed with brine, dried over anhydrous MgSO₄, filtered and concentrated under reduced pressure. The crude residue was purified by flash column chromatography (dichloromethane:MeOH=6:1) to obtain the desired product JGJ004 (21.1 mg, 0.079 mmol, 89%) as an ivory solid. ¹H NMR (400 MHz, CDCl₃) δ 8.32 (s, 1H), 8.09 (d, J=9.6 Hz, 1H), 7.88 (br s, 1H), 7.70 (d, J=7.6 Hz, 1H), 7.65 (d, J=8.0 Hz, 1H), 7.54 (d, J=10.0 Hz, 1H), 7.48 (t, J=8.0 Hz, 1H), 2.86 (s, 3H), 2.25 (s, 3H). ¹³C NMR (125 MHz, CD₃OD) δ 172.8, 156.1, 149.9, 145.8, 141.8, 137.0, 131.5, 126.3, 124.8, 124.2, 122.6, 120.5, 24.8, 10.4.

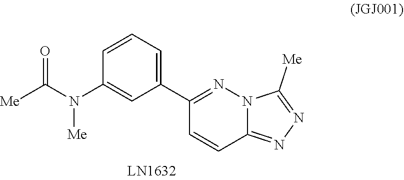 [SMILES: CC(=O)N(C)c1cccc(-c2ccc3nnc(C)n3n2)c1]

N-Methyl-N-(3-(3-methyl-[1,2,4]triazolo[4,3-b]pyridazin-6-yl)phenyl)acetamide, JGJ001. To a solution of N-(3-(3-methyl-[1,2,4]triazolo[4,3-b]pyridazin-6-yl)phenyl)acetamide (JGJ004, 16.5 mg, 0.062 mmol) was added NaH, 60% dispersion in mineral oil (5 mg, 0.124 mmol) at 0° C. and it was stirred for 30 min. Then iodomethane (17.5 mg, 0.124 mmol) was added and the reaction mixture was stirred at 23° C. for 2 h. After the reaction was completed, water was added and it was extracted with EtOAc. The combined organic layer was washed with brine, dried over anhydrous MgSO₄, filtered and concentrated under reduced pressure. The crude residue was purified by flash column chromatography (dichloromethane:MeOH=10:1) to obtain the desired product JGJ001 (9.8 mg, 0.035 mmol, 56%) as an ivory solid. ¹H NMR (500 MHz, CDCl₃) δ 8.17 (d, J=9.5 Hz, 1H), 7.95 (d, J=7.5 Hz, 1H), 7.88 (s, 1H), 7.62 (dd, J=8.0, 7.5 Hz, 1H), 7.54 (d, J=10.0 Hz, 1H), J=8.0 Hz, 1H), 3.35 (s, 3H), 2.89 (s, 3H), 1.94 (s, 3H); ¹³C NMR (125 MHz, CDCl₃) δ 170.3, 152.1, 147.6, 145.6, 143.3, 136.2, 130.7, 129.5, 126.4, 125.9, 125.4, 118.4, 37.3, 22.6, 9.9.

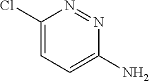

6-Chloropyridazin-3-amine. A mixture of 3,6-dichloropyridazine (200 mg, 2.342 mmol) and ammonium hydroxide (1.5 mL) in a sealed tube was heated 100° C. for 16 h. After the mixture was cooled to 23° C., dichloromethane was added and the precipitate was isolated and washed with dichloromethane to obtain the desired product (quant.) as a light yellow solid. ¹H NMR (400 MHz, DMSO-d₆) δ 7.32 (d, J=8.0 Hz, 1H), 6.81 (d, J=8.0 Hz, 1H), 6.59 (s, 2H).

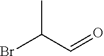

2-Bromopropionaldehyde. To a solution of propionaldehyde (2.91 mL, 40 mol) in dichloromethane (40 mL) was added dropwise bromine (2.05 mL, 40 mol) in dichloromethane (10 mL) at 0° C. over 1.5 h. The mixture was warmed to 23° C. and stirred for 30 min. After water was added to the reaction, the resulting organic layer was separated and washed with saturated sodium bicarbonate solution. The aqueous layer was extracted with dichloromethane (30 mL) and then the combined organic layer was washed with brine, dried over anhydrous MgSO₄, filtered and concentrated under reduced pressure. The crude product (dark yellow oil, quant.) was used for the next step without any purification. ¹H NMR (400 MHz, CDCl₃) δ 9.35 (br s, 1H), 4.34 (qd, J=6.8, 2.0 Hz, 1H), 1.75 (d, J=6.8 Hz, 3H). The spectroscopic data match the literature data. [Ref: Bull. Korean Chem. Soc. 2013, 34(1), 271-274.

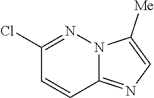 [SMILES: Cc1cnc2ccc(Cl)nn12]

6-Chloro-3-methylimidazo[1,2-b]pyridazine. A mixture of 6-chloropyridazin-3-amine (500 mg, 3.860 mmol) and 2-bromopropionaldehyde (crude, 793 mg, 5.789 mmol) in EtOH (10 mL) was heated at reflux for 4 h. After the mixture was cooled to 23° C., it was concentrated and extracted with EtOAc. The combined organic layer was washed with brine, dried over anhydrous MgSO₄, filtered and concentrated under reduced pressure. The crude residue was purified by flash column chromatography (dichloromethane:MeOH=15:1) to obtain the desired product (172 mg, 1.026 mmol, 27%) as a light brown solid. ¹H NMR (400 MHz, CDCl₃) δ 7.87 (d, J=9.6 Hz, 1H), 7.56 (s, 1H), 6.99 (1H, J=9.6 Hz, 1H), 2.55 (s, 3H). The spectroscopic data match the literature data. [Ref: Chem. Pharm. Bull. 1996, 44(1), 122-131.

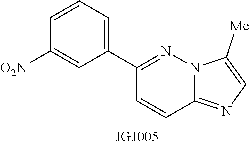 [SMILES: Cc1cnc2ccc(-c3cccc([N+](=O)[O-])c3)nn12]

3-Methyl-6-(3-nitrophenyl)imidazo[1,2-b]pyridazine, JGJ005. The reaction of 6-chloro-3-methylimidazolo[1,2-b]pyridazine (55.2 mg, 0.329 mmol), 3-nitrophenylboronic acid (60.5 mg, 0.362 mmol), K₂CO₃ (68.3 mg, 0.494 mmol) and Pd(PPh₃)₄ (38.1 mg, 0.033 mmol) in 1,4-dioxane (0.5 mL) and water (150 μL) afforded the desired product JGJ005 (61.9 mg, 0.244 mmol, 74%) as a yellow solid using the same procedure as described for JGJ002. ¹H NMR (500 MHz, CDCl₃) δ 8.88 (dd, J=2.0, 1.5 Hz, 1H), 8.38 (ddd, J=7.5, 1.5, 1.0 Hz, 1H), 8.35 (ddd, J=8.0, 2.0, 1.0 Hz, 1H), 8.07 (d, J=9.5 Hz, 1H), 7.73 (t, J=8.0 Hz, 1H), 7.67 (s, 1H), 7.50 (d, J=9.5 Hz, 1H), 2.67 (s, 3H); ¹³C NMR (125 MHz, CDCl₃) δ 148.8 (two peaks are overlapped), 138.1, 137.7, 133.3, 132.7, 130.0, 126.0, 125.8, 124.4, 122.0, 113.7, 8.8.

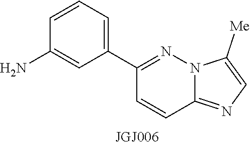 [SMILES: Cc1cnc2ccc(-c3cccc(N)c3)nn12]

3-(3-Methylimidazo[1,2-b]pyridazin-6-yl)aniline, JGJ006. A reaction of 3-methyl-6-(3-nitro-phenyl)imidazo[1,2-b]pyridazine (54.4 mg, 0.214 mmol) and SnCl₂ (202.8 mg, 1.070 mmol) in EtOH (0.5 mL) afforded the desired product JGJ006 (27.2 mg, 0.107 mmol, 50%) as a light yellow solid using the same procedure as described for JGJ003. ¹H NMR (400 MHz, CDCl₃) δ 7.92 (d, J=9.2 Hz, 1H), 7.56 (d, J=0.8 Hz, 1H), 7.38 (d, J=9.6 Hz, 1H), 7.28-7.34 (m, 3H), 6.79 (ddd, J=7.6, 2.0, 1.2 Hz, 1H), 3.86 (br s, 2H), 2.61 (d, J=0.8 Hz, 3H); ¹³C NMR (100 MHz, CDCl₃) δ 151.3, 147.0, 138.1, 137.0, 132.0, 129.8, 125.3, 125.1, 117.3, 116.5, 114.8, 113.3, 8.7.

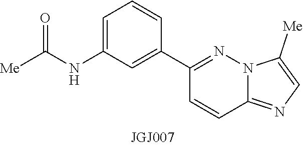 [SMILES: CC(=O)Nc1cccc(-c2ccc3ncc(C)n3n2)c1]

N-(3-(3-Methylimidazo[1,2-b]pyridazin-6-yl)phenyl)acetamide, JGJ007. The reaction of 3-(3-methylimidazo[1,2-b]pyridazin-6-yl)aniline (JGJ006, 23.3 mg, 0.104 mmol), triethylamine (12.6 mg, 0.125 mmol) and acetyl chloride (9 mg, 0.114 mmol) in dichloromethane (0.5 mL) afforded the desired product JGJ007 (16.5 mg, 0.067 mmol, 60%) as an ivory solid using the same procedure as described for JGJ004. ¹H NMR (400 MHz, CDCl₃) δ 9.12 (br s, NH), 8.23 (s, 1H), 7.82 (d, J=9.6 Hz, 1H), 7.61-7.69 (m, 2H), 7.53 (s, 1H), 7.36 (t, J=8.0 Hz, 1H), 7.30 (d, J=9.6 Hz, 1H), 2.51 (s, 3H), 2.21 (s, 3H); ¹³C NMR (100 MHz, CDCl₃) δ 169.2, 150.8, 139.1, 137.8, 136.3, 131.7, 129.4, 125.4, 124.8, 122.4, 121.2, 118.3, 114.7, 24.4, 8.5.

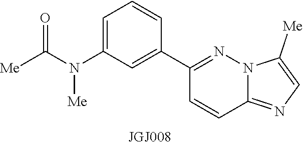 [SMILES: CC(=O)N(C)c1cccc(-c2ccc3ncc(C)n3n2)c1]

N-Methyl-N-(3-(3-methylimidazo[1,2-b]pyridazin-6-yl)phenyl)acetamide, JGJ008. The reaction of N-(3-(3-methylimidazo[1,2-b]pyridazin-6-yl)phenyl)acetamide (JGJ007, 26.4 mg, 0.099 mmol), NaH, 60% dispersion in mineral oil (8 mg, 0.199 mmol) and iodomethane (28.2 mg, 0.199 mmol) in dimethylformamide (DMF, 0.3 mL) afforded the desired product JGJ008 (17.5 mg, 0.062 mmol, 63%) as an ivory solid using the same procedure as described for JGJ001. ¹H NMR (400 MHz, CDCl₃) δ 8.00 (d, J=9.6 Hz, 1H), 7.96 (d, J=8.0 Hz, 1H), 7.89 (dd, J=2.0, 1.6 Hz, 1H), 7.62 (s, 1H), 7.58 (dd, J=8.0, 7.6 Hz, 1H), 7.43 (d, J=9.2 Hz, 1H), 7.32 (dd, J=7.6, 1.2 Hz, 1H), 3.34 (s, 3H), 2.64 (s, 3H), 1.95 (s, 3H); ¹³C NMR (125 MHz, CDCl₃) δ 170.5, 149.9, 145.4, 138.1, 137.8, 132.8, 130.4, 128.3, 126.2, 125.7, 125.6, 114.0, 37.2, 22.6, 8.8 (one low-field carbon not observed).

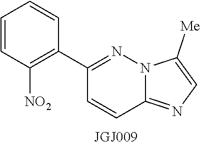 [SMILES: Cc1cnc2ccc(-c3ccccc3[N+](=O)[O-])nn12]

3-Methyl-6-(2-nitrophenyl)imidazo[1,2-b]pyridazine, JGJ009. The reaction of 6-chloro-3-methylimidazolo[1,2-b]pyridazine (67.1 mg, 0.400 mmol), 2-nitrophenylboronic acid (73.5 mg, 0.440 mmol), NaOH (48 mg, 1.201 mmol) and Pd(PPh₃)₄ (46.3 mg, 0.040 mmol) in THE (0.4 mL) and water (0.2 mL) at 80° C. afforded the desired product JGJ009 (16.3 mg, 0.064 mmol, 16%) as a yellow solid using the same procedure as described for JGJ002. ¹H NMR (400 MHz, CDCl₃) 8.02 (dd, J=8.0, 0.8 Hz, 1H), 7.99 (d, J=9.6 Hz, 1H), 7.75 (m, 1H), 7.64-7.70 (m, 2H), 7.63 (d, J=1.2 Hz, 1H), 7.10 (d, J=9.2 Hz, 1H), 2.54 (s, 3H); ¹³C NMR (100 MHz, CDCl₃) δ 149.6, 149.0, 137.7, 132.9, 132.8, 131.7, 131.4, 130.2, 125.6, 125.5, 124.7, 115.8, 8.6.

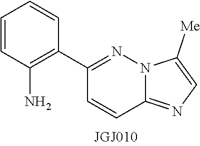 [SMILES: Cc1cnc2ccc(-c3ccccc3N)nn12]

2-(3-Methylimidazo[1,2-b]pyridazin-6-yl)aniline, JGJ010. The reaction of 6-chloro-3-methylimidazolo[1,2-b]pyridazine (25.4 mg, 0.152 mmol), 2-aminophenylboronic acid (22.8 mg, 0.167 mmol), K₂CO₃ (31.4 mg, 0.227 mmol) and Pd(PPh₃)₄ (17.5 mg, 0.015 mmol) in 1,4-dioxane (0.4 mL) and water (80 μL) at 110° C. afforded the desired product JGJ010 (26.2 mg, 0.117 mmol, 70%) as a pale yellow solid using the same procedure as described for JGJ002. ¹H NMR (400 MHz, CDCl₃) 7.97 (d, J=9.6 Hz, 1H), 7.57 (s, 1H), 7.67 (m, 1H), 7.42 (d, J=9.6 Hz, 1H), 7.24 (m, 1H), 6.82-6.87 (m, 2H), 2.59 (s, 3H); ¹³C NMR (100 MHz, CDCl₃) δ 152.8, 145.9, 137.3, 131.8, 130.7, 129.7, 125.6, 124.9, 118.6, 118.0, 117.4, 116.5, 8.8.

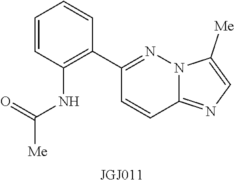 [SMILES: CC(=O)Nc1ccccc1-c1ccc2ncc(C)n2n1]

N-(2-(3-Methylimidazo[1,2-b]pyridazin-6-yl)phenyl)acetamide, JGJ011. The reaction of 2-(3-methylimidazo[1,2-b]pyridazin-6-yl)aniline (JGJ010, 39.4 mg, 0.176 mmol), triethylamine (21.3 mg, 0.211 mmol) and acetyl chloride (16.5 mg, 0.211 mmol) in dichloromethane (0.8 mL) afforded the desired product JGJ011 (35 mg, 0.131 mmol, 75%) as an ivory solid using the same procedure as described for JGJ004. ¹H NMR (400 MHz, CDCl₃) δ 10.57 (br s, NH), 8.47 (d, J=8.4 Hz, 1H), 7.99 (d, J=9.6 Hz, 1H), 7.61 (s, 1H), 7.60 (dd, J=8.0, 0.8 Hz, 1H), 7.44 (ddd, J=8.8, 7.2, 0.8 Hz, 1H), 7.34 (d, J=9.2 Hz, 1H), 7.20 (ddd, J=8.0, 7.2, 0.8 Hz, 1H), 2.60 (s, 3H), 2.17 (s, 3H); ¹³C NMR (100 MHz, CDCl₃) δ 168.1, 152.0, 137.3, 136.4, 132.8, 130.6, 129.5, 126.3, 124.6, 124.0, 123.5, 122.4, 116.7, 25.1, 8.9.

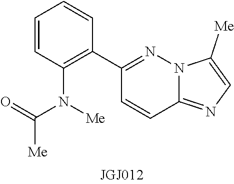 [SMILES: CC(=O)N(C)c1ccccc1-c1ccc2ncc(C)n2n1]

N-Methyl-N-(2-(3-methylimidazo[1,2-b]pyridazin-6-yl)phenyl)acetamide, JGJ012. The reaction of N-(2-(3-methylimidazo[1,2-b]pyridazin-6-yl)phenyl)acetamide (JGJ011, 19.1 mg, 0.072 mmol), sodium hydride (NaH, 60% dispersion in mineral oil, 5.7 mg, 0.143 mmol) and iodomethane (20.4 mg, 0.143 mmol) in dimethylformamide (DMF, 0.3 mL) afforded the desired product JGJ012 (12.8 mg, 0.046 mmol, 64%) as an ivory solid using the same procedure as described for JGJ001. ¹H NMR (400 MHz, CDCl₃) δ 7.98 (d, J=9.2 Hz, 1H), 7.66 (m, 1H), 7.60 (s, 1H) 7.52 (m, 2H), 7.34 (m, 1H), 7.10 (d, J=9.6 Hz, 1H), 3.01 (s, 3H), 2.54 (s, 3H), 1.90 (s, 3H); ¹³C NMR (100 MHz, CDCl₃) δ 170.9, 150.1, 142.5, 137.4, 134.5, 132.8, 131.0, 130.9, 130.7, 129.5, 128.7, 125.7, 116.0, 36.7, 22.7, 8.7.

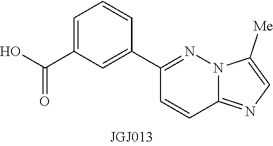 [SMILES: Cc1cnc2ccc(-c3cccc(C(=O)O)c3)nn12]

3-(3-Methylimidazo[1,2-b]pyridazin-6-yl)benzoic acid, JGJ013. The reaction of 6-chloro-3-methylimidazolo[1,2-b]pyridazine (50 mg, 0.299 mmol), 3-carboxyphenylboronic acid (54.5 mg, 0.328 mmol), K₂CO₃ (82.5 mg, 0.597 mmol) and Pd(PPh₃)₄ (34.5 mg, 0.030 mmol) in 1,4-dioxane (0.5 mL) and water (100 μL) afforded the desired product JGJ013 (32.4 mg, 0.128 mmol, 43%) as a white solid using the same procedure as described for JGJ002. ¹H NMR (400 MHz, CD₃OD) 8.73 (dd, J=1.6, 1.2 Hz, 1H), 8.25 (d, J=8.0 Hz, 1H), 8.16 (ddd, J=7.6, 1.6, 1.2 Hz, 1H), 8.03 (d, J=9.6 Hz, 1H), 7.75 (d, J=9.6 Hz, 1H), 7.62 (dd, J=8.0, 7.6 Hz, 1H), 7.58 (d, J=0.4 Hz, 1H), 2.63 (d, J=0.4 Hz, 3H).

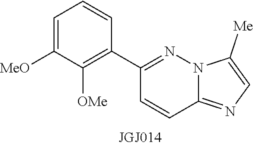 [SMILES: COc1cccc(-c2ccc3ncc(C)n3n2)c1OC]

6-(2,3-Dimethoxyphenyl)-3-methylimidazo[1,2-b]pyridazine, JGJ014. The reaction of 6-chloro-3-methylimidazolo[1,2-b]pyridazine (42 mg, 0.251 mmol), 2,3-dimethoxyphenylboronic acid (50.2 mg, 0.276 mmol), K₂CO₃ (52 mg, 0.376 mmol) and Pd(PPh₃)₄ (29 mg, 0.025 mmol) in 1,4-dioxane (0.5 mL) and water (100 μL) afforded the desired product JGJ014 (39.6 mg, 0.147 mmol, 59%) as an ivory solid using the same procedure as described for JGJ002. ¹H NMR (400 MHz, CDCl₃) 7.92 (d, J=9.6 Hz, 1H), 7.58 (s, 1H), 7.46 (d, J=9.2 Hz, 1H), 7.29 (dd, J=7.6, 0.8 Hz, 1H), 7.19 (t, J=8.0 Hz, 1H), 7.05 (ddd, J=8.0, 7.6, 0.8 Hz, 1H), 3.93 (s, 3H), 3.76 (s, 3H), 2.60 (s, 3H); ¹³C NMR (100 MHz, CDCl₃) δ 153.2, 150.7, 147.5, 138.0, 131.7, 131.1, 125.2, 124.4, 124.2, 122.2, 118.4, 113.6, 61.4, 56.0, 8.8.

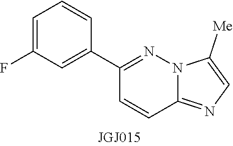 [SMILES: Cc1cnc2ccc(-c3cccc(F)c3)nn12]

6-(3-Fluorophenyl)-3-methylimidazo[1,2-b]pyridazine, JGJ015. The reaction of 6-chloro-3-methylimidazolo[1,2-b]pyridazine (51.5 mg, 0.307 mmol), 3-fluorophenylboronic acid (47.3 mg, 0.338 mmol), K₂CO₃ (63.7 mg, 0.461 mmol) and Pd(PPh₃)₄ (35.5 mg, 0.031 mmol) in 1,4-dioxane (0.5 mL) and water (100 μL) afforded the desired product JGJ015 (38.2 mg, 0.168 mmol, 55%) as an ivory solid using the same procedure as described for JGJ002. ¹H NMR (400 MHz, CDCl₃) 7.98 (d, J=9.2 Hz, 1H), 7.75 (m, 2H), 7.61 (s, 1H), 7.48 (m, 1H), 7.41 (d, J=9.2 Hz, 1H), 7.18 (m, 1H), 2.63 (s, 3H); ¹³C NMR (100 MHz, CDCl₃) δ 163.2 (d, J=244.9 Hz), 149.8 (d, J=2.6 Hz), 138.2, 138.1, 132.6, 130.5 (d, J=8.1 Hz), 125.5, 122.6 (d, J=2.9 Hz), 116.7 (d, J=21.2 Hz), 114.2, 113.9 (d, J=23.1 Hz), 8.7. (one low-field carbon not observed).

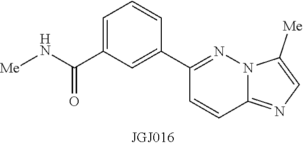 [SMILES: CNC(=O)c1cccc(-c2ccc3ncc(C)n3n2)c1]

N-Methyl-3-(3-methylimidazo[1,2-b]pyridazin-6-yl)benzamide, JGJ016. To a solution of JGJ013 (20.1 mg, 0.079 mmol) and methylamine hydrochloride (10.7 mg, 0.159 mmol) in dichloromethane (0.3 mL) and DMF (0.5 mL) was added hydroxybenzotriazole (HOBT, 16.1 mg, 0.159 mmol), (3-dimethylaminopropyl)-N′-ethylcarbodiimide hydrochloride, (EDC.HCl, 30.4 mg, 0.159 mmol) and N,N-diisopropylethylamine (DIPEA, 102.6 mg, 0.794 mmol). The mixture was stirred at 23° C. for 12 h. After water was added to the reaction, it was extracted with ethyl acetate (10 mL×3). The combined organic layer was washed with brine, dried over anhydrous MgSO₄, filtered and concentrated under reduced pressure. The crude residue was purified by flash column chromatography (dichloromethane:MeOH=6:1) to obtain the desired product JGJ016 (8.6 mg, 0.032 mmol, 41%) as a pale yellow solid. ¹H NMR (400 MHz, CDCl₃) δ 8.39 (t, J=1.6 Hz, 1H), 8.10 (dddd, J=8.0, 1.6, 1.2, 0.8 Hz, 1H), 7.91 (d, J=9.6 Hz, 1H), 7.86 (ddd, J=7.6, 1.6, 1.2 Hz, 1H), 7.58 (s, 1H), 7.55 (dd, J=8.0, 7.6 Hz, 1H), 7.41 (d, J=9.6 Hz, 1H), 6.75 (m, NH), 3.06 (d, J=4.8 Hz, 3H), 2.59 (d, J=0.4 Hz, 3H); ¹³C NMR (100 MHz, CDCl₃) δ 167.7, 153.3, 138.0, 136.3, 135.5, 132.3, 129.7, 129.2, 128.0, 125.7, 125.5, 125.4, 114.4, 26.9, 8.7.

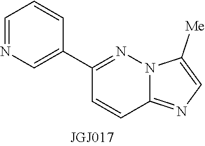 [SMILES: Cc1cnc2ccc(-c3cccnc3)nn12]

3-Methyl-6-(pyridin-3-yl)imidazo[1,2-b]pyridazine, JGJ017. The reaction of 6-chloro-3-methylimidazolo[1,2-b]pyridazine (58.8 mg, 0.351 mmol), 3-pyridineboronic acid (47.4 mg, 0.386 mmol), K₂CO₃ (72.7 mg, 0.526 mmol) and Pd(PPh₃)₄ (40.6 mg, 0.035 mmol) in 1,4-dioxane/water (5:1 v/v, 0.6 mL) afforded the desired product JGJ017 (37.2 mg, 0.177 mmol, 50%) as a pale yellow solid using the same procedure as described for JGJ002. ¹H NMR (400 MHz, CDCl₃) 9.20 (d, J=1.6 Hz, 1H), 8.69 (dd, J=4.8, 1.6 Hz, 1H), 8.29 (ddd, J=8.0, 2.0, 1.6 Hz, 1H), 7.98 (d, J=9.2 Hz, 1H), 7.60 (d, J=0.4 Hz, 1H), 7.42 (ddd, J=8.0, 4.8, 0.8 Hz, 1H), 7.41 (d, J=9.6 Hz, 1H), 2.60 (d, J=0.8 Hz, 3H); ¹³C NMR (100 MHz, CDCl₃) δ 150.6, 148.6, 148.2, 137.9, 134.2, 132.7, 131.6, 125.7, 125.5, 123.6, 113.7, 8.6.

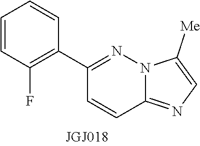 [SMILES: Cc1cnc2ccc(-c3ccccc3F)nn12]

6-(2-Fluorophenyl)-3-methylimidazo[1,2-b]pyridazine, JGJ018. The reaction of 6-chloro-3-methylimidazolo[1,2-b]pyridazine (27.5 mg, 0.164 mmol), 2-fluorophenylboronic acid (25.3 mg, 0.181 mmol), K₂CO₃ (34.0 mg, 0.246 mmol) and Pd(PPh₃)₄ (19.0 mg, 0.016 mmol) in 1,4-dioxane/water (5:1 v/v, 0.5 mL) afforded the desired product JGJ018 (18.1 mg, 0.080 mmol, 49%) as an ivory solid using the same procedure as described for JGJ002. ¹H NMR (400 MHz, CDCl₃) 7.96 (d, J=9.6 Hz, 1H), 7.91 (ddd, J=8.0, 7.6, 2.0 Hz, 1H), 7.60 (s, 1H), 7.43-7.49 (m, 2H), 7.30 (ddd, J=8.0, 7.6, 1.2 Hz, 1H), 7.21 (ddd, J=11.2, 8.4, 0.8 Hz, 1H), 2.61 (d, J=0.8 Hz, 3H); ¹³C NMR (100 MHz, CDCl₃) δ 160.4 (d, J=249.3 Hz), 148.2, 137.9, 132.2, 131.4 (d, J=8.5 Hz), 130.7 (d, J=2.6 Hz), 125.3, 124.7, 124.6 (d, J=3.6 Hz), 124.3 (d, J=11.7 Hz), 117.5 (d, J=7.9 Hz), 116.4 (d, J=22.2 Hz), 8.7.

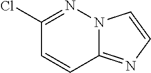

6-Chloroimidazo[1,2-b]pyridazine. To a solution of 6-chloropyridazin-3-amine (400 mg, 3.088 mmol) in EtOH (6 mL) and water (4 mL) was added bromoacetaldehyde diethyl acetal (930 μL, 6.175 mmol) and HBr (280 μL). The resulting mixture was heated at 103° C. overnight. After it was cooled to 23° C., the mixture was diluted water and extracted with EtOAc. The combined organic layer was washed with saturated NaHCO₃ solution, dried over anhydrous MgSO₄, filtered and concentrated under reduced pressure. The resulting crude residue was used for the next step without further purification. (Brown solid; 394.5 mg, 2.569 mmol, 83%)¹H NMR (400 MHz, CDCl₃) δ 7.92 (s, 1H), 7.90 (d, J=9.6 Hz, 1H), 7.76 (s, 1H), 7.04 (d, J=9.6 Hz, 1H); ¹³C NMR (100 MHz, CDCl₃) δ 146.9, 137.5, 134.4, 127.0, 118.9, 117.2.

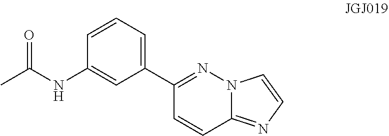 [SMILES: CC(=O)Nc1cccc(-c2ccc3nccn3n2)c1]

N-(3-(Imidazo[1,2-b]pyridazin-6-yl)phenyl)acetamide, JGJ019. The reaction of 6-chloro-imidazo[1,2-b]pyridazine (71.6 mg, 0.427 mmol), 3-aminophenylboronic acid (69.5 mg, 0.449 mmol), K₂CO₃ (88.6 mg, 0.641 mmol) and Pd(PPh₃)₄ (49.3 mg, 0.043 mmol) in 1,4-dioxane/water (5:1 v/v, 1.0 mL) afforded 3-(imidazo[1,2-b]pyridazin-6-yl)aniline (87.9 mg, 0.392 mmol, 92%) as a light yellow solid using the same procedure as described for JGJ002. Then the acetylation using the same procedure as described for JGJ004 gave the desired product JGJ019 (49.6 mg, 0.221 mmol, 69%) as an ivory solid. ¹H NMR (400 MHz, CDCl₃) δ 8.19 (s, 1H), 8.13 (br s, 1H), 7.96 (m, 2H), 7.76 (s, 1H), 7.61-7.65 (m, 2H), 7.43 (d, J=9.6 Hz, 1H), 7.39-7.43 (m, 1H), 2.22 (s, 3H); ¹³C NMR (100 MHz, CDCl₃) δ 168.9, 151.8, 138.9, 138.2, 136.1, 133.6, 129.7, 125.4, 122.7, 121.4, 118.4, 117.1, 116.7, 24.6.

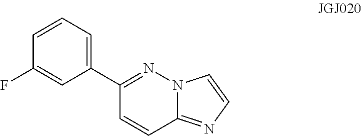 [SMILES: Fc1cccc(-c2ccc3nccn3n2)c1]

6-(3-Fluorophenyl)imidazo[1,2-b]pyridazine, JGJ020. The reaction of 6-chloroimidazo[1,2-b]pyridazine (50 mg, 0.326 mmol), 3-fluorophenylboronic acid (50.1 mg, 0.358 mmol), K₂CO₃ (67.5 mg, 0.488 mmol) and Pd(PPh₃)₄ (18.8 mg, 0.016 mmol) in 1,4-dioxane/water (5:1 v/v, 0.5 mL) afforded the desired product JGJ020 (36.9 mg, 0.173 mmol, 53%) as an ivory solid using the same procedure as described for JGJ002. ¹H NMR (400 MHz, CDCl₃) δ 7.96-7.99 (m, 2H), 7.77 (s, 1H), 7.62-7.68 (m, 2H), 7.42-7.46 (m, 1H), 7.39 (d, J=9.6 Hz, 1H), 7.14 (m, 1H); ¹³C NMR (100 MHz, CDCl₃) δ 163.1 (d, J=245.1 Hz), 150.4 (d, J=2.6 Hz), 137.5 (d, J=7.8 Hz), 134.2, 131.9 (d, J=9.8 Hz), 130.5 (d, J=8.1 Hz), 128.4 (d, J=12.1 Hz), 125.7, 122.5 (d, J=2.9 Hz), 116.8 (d, J=21.1 Hz), 115.7, 113.8 (d, J=23.2 Hz).

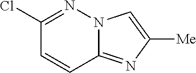 [SMILES: Cc1cn2nc(Cl)ccc2n1]

6-Chloro-2-methylimidazo[1,2-b]pyridazine. To a solution of 6-chloropyridazin-3-amine (100 mg, 0.772 mmol) in EtOH (2 mL) was added trimethylamine (78 mg, 0.772 mmol) and chloro-acetone (142.8 mg, 1.544 mmol) and the mixture was stirred at 120° C. overnight. After the mixture was cooled to 23° C., it was diluted with water and extracted with EtOAc. The combined organic layer was washed with brine, dried over anhydrous MgSO₄, filtered and concentrated under reduced pressure. The crude residue was purified by flash column chromatography (n-Hexane:EtOAc=1:1) to obtain the desired product (87.2 mg, 0.520 mmol, 67%) as off-white solid. ¹H NMR (400 MHz, CDCl₃) δ 7.72 (dd, J=9.2, 0.4 Hz, 1H), 7.65 (s, 1H), 6.93 (d, J=9.2 Hz, 1H), 2.44 (d, J=0.8 Hz, 3H); ¹³C NMR (100 MHz, CDCl₃) δ 145.8, 144.8, 137.0, 125.6, 117.9, 114.5, 14.7.

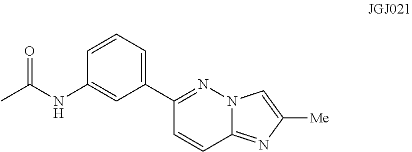 [SMILES: CC(=O)Nc1cccc(-c2ccc3nc(C)cn3n2)c1]

N-(3-(2-Methylimidazo[1,2-b]pyridazin-6-yl)phenyl)acetamide, JGJ021. The reaction of 6-chloro-2-methylimidazo[1,2-b]pyridazine (35.3 mg, 0.211 mmol), 3-aminophenylboronic acid (35.9 mg, 0.232 mmol), K₂CO₃ (43.7 mg, 0.316 mmol) and Pd(PPh₃)₄ (24.4 mg, 0.021 mmol) in 1,4-dioxane/water (5:1 v/v, 0.5 mL) afforded 3-(2-methylimidazo[1,2-b]pyridazin-6-yl)aniline (49.6 mg, quant.) as an pale yellow solid using the same procedure as described for JGJ002. Then the acetylation using the same procedure as described for JGJ004 gave the desired product JGJ021 (27.2 mg, 0.102 mmol, 46%) as an ivory solid. ¹H NMR (400 MHz, CDCl₃) δ 8.92 (s, 1H), 8.16 (s, 1H), 7.73 (d, J=9.6 Hz, 1H), 7.63 (m, 2H), 7.54 (d, J=7.6 Hz, 1H), 7.33 (t, J=8.0 Hz, 1H), 7.27 (d, J=10.0 Hz, 1H), 2.44 (s, 3H), 2.19 (s, 3H); ¹³C NMR (100 MHz, CDCl₃) δ 169.2, 150.7, 143.8, 139.0, 137.7, 136.1, 129.4, 123.9, 122.3, 121.1, 118.2, 115.7, 114.3, 24.4, 14.5.

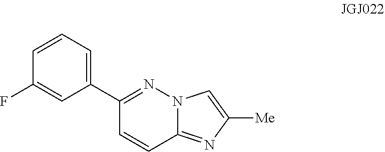 [SMILES: Cc1cn2nc(-c3cccc(F)c3)ccc2n1]

6-(3-Fluorophenyl)-2-methylimidazo[1,2-b]pyridazine, JGJ022. The reaction of 6-chloro-2-methylimidazo[1,2-b]pyridazine (21.4 mg, 0.128 mmol), 3-fluorophenylboronic acid (17.9 mg, 0.128 mmol), K₂CO₃ (26.5 mg, 0.192 mmol) and Pd(PPh₃)₄ (7.4 mg, 0.006 mmol) in 1,4-dioxane/water (5:1 v/v, 0.3 mL) afforded the desired product JGJ022 (13.7 mg, 0.060 mmol, 47%) as an ivory solid using the same procedure as described for JGJ002. ¹H NMR (400 MHz, CDCl₃) δ 7.89 (d, J=9.2 Hz, 1H), 7.78 (s, 1H), 7.65-7.70 (m, 2H), 7.43-7.49 (m, 1H), 7.38 (d, J=9.2 Hz, 1H), 7.16 (m, 1H), 2.52 (d, J=0.4 Hz, 3H); ¹³C NMR (100 MHz, CDCl₃) δ 163.2 (d, J=245.0 Hz), 149.8 (d, J=2.7 Hz), 144.5, 137.9 (d, J=8.0 Hz), 130.5 (d, J=8.2 Hz), 124.5, 122.5 (d, J=3.0 Hz), 116.7 (d, J=21.1 Hz), 115.3, 114.4, 113.9 (d, J=23.2 Hz), 14.8. (one low-field carbon not observed)

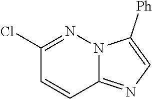 [SMILES: Clc1ccc2ncc(-c3ccccc3)n2n1]

6-Chloro-3-phenylimidazo[1,2-b]pyridazine. To a solution of 6-chloroimidazo[1,2-b]pyridazine (394.5 mg, 2.569 mmol) in DMF (6 mL) was added N-iodosuccinimide (635.8 mg, 2.826 mmol) and the mixture was stirred at 23° C. for 48 h. After the reaction was completed, it was vacuumed to remove the solvent. The residue was diluted with dichloromethane and washed with saturated Na₂S₂CO₃ solution. The organic layer was separated and washed with brine, dried over anhydrous MgSO₄, filtered and concentrated under reduced pressure to give 6-chloro-3-iodoimidazo[1,2-b]pyridazine in quantitative yield. Then a mixture of 6-chloro-3-iodoimidazo[1,2-b]pyridazine (107.2 mg, 0.326 mmol), phenylboronic acid (43.7 mg, 0.358 mmol), K₂CO₃ (54.0 mg, 0.391 mmol) and Pd(PPh₃)₄ (18.8 mg, 0.016 mmol) in 1,4-dioxane/water (5:1 v/v, 2 mL) was heated at 90° C. overnight. After the reactant was cooled to 23° C., it was diluted in water and extracted with EtOAc. The combined organic layer was washed with brine, dried over anhydrous MgSO₄, filtered and concentrated under reduced pressure. The crude residue was purified by flash column chromatography (n-Hexane:EtOAc=2:1) to obtain the desired product (28.4 mg, 0.124 mmol, 38%) as a pale yellow solid. ¹H NMR (400 MHz, CDCl₃) δ 8.06 (s, 1H), 8.03 (m, 2H), 7.98 (d, J=9.6 Hz, 1H), 7.52 (m, 2H), 7.39 (m, 1H), 7.08 (d, J=9.2 Hz, 1H); ¹³C NMR (100 MHz, CDCl₃) δ 146.8, 138.5, 133.1, 129.1, 128.7, 128.4, 127.6, 127.1, 126.8, 118.3.

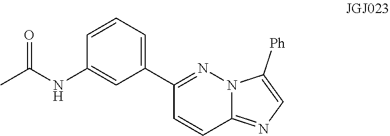 [SMILES: CC(=O)Nc1cccc(-c2ccc3ncc(-c4ccccc4)n3n2)c1]

N-(3-(3-Phenylimidazo[1,2-b]pyridazin-6-yl)phenyl)acetamide, JGJ023. The reaction of 6-chloro-3-phenylimidazo[1,2-b]pyridazine (15.5 mg, 0.068 mmol), 3-aminophenylboronic acid (11.5 mg, 0.074 mmol), K₂CO₃ (14.0 mg, 0.101 mmol) and Pd(PPh₃)₄ (3.9 mg, 0.003 mmol) in 1,4-dioxane/water (5:1 v/v, 0.2 mL) afforded 3-(3-phenylimidazo[1,2-b]pyridazin-6-yl)aniline (17.5 mg, 0.061 mmol, 91%) as an pale yellow solid using the same procedure as described for JGJ002. Then the acetylation using the same procedure as described for JGJ004 gave the desired product JGJ023 (10.9 mg, 0.033 mmol, 54%) as an ivory solid. ¹H NMR (400 MHz, CDCl₃) δ 8.18 (s, 1H), 8.12 (m, 2H), 8.04 (s, 1H), 7.99 (d, J=9.6 Hz, 1H), 7.93 (br s, 1H), 7.64-7.70 (m, 2H), 7.50 (m, 2H), 7.46 (d, J=9.6 Hz, 1H), 7.35-7.44 (m, 2H), 2.22 (s, 3H); ¹³C NMR (100 MHz, CDCl₃) δ 168.7, 151.1, 138.8, 136.4, 133.0, 129.6, 128.8, 128.7, 128.6, 127.9, 126.8, 125.8, 122.7, 121.3, 118.3, 115.6, 24.6. (one low-field carbon not observed)

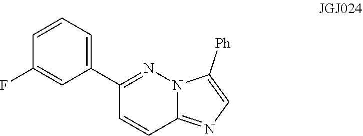 [SMILES: Fc1cccc(-c2ccc3ncc(-c4ccccc4)n3n2)c1]

6-(3-Fluorophenyl)-3-phenylimidazo[1,2-b]pyridazine, JGJ024. The reaction of 6-chloro-3-phenylimidazo[1,2-b]pyridazine (12.9 mg, 0.056 mmol), 3-fluorophenylboronic acid (8.6 mg, 0.062 mmol), K₂CO₃ (11.7 mg, 0.084 mmol) and Pd(PPh₃)₄ (3.2 mg, 0.003 mmol) in 1,4-dioxane/water (5:1 v/v, 0.2 mL) afforded the desired product JGJ024 (9.5 mg, 0.033 mmol, 58%) as an ivory solid using the same procedure as described for JGJ002. ¹H NMR (400 MHz, CDCl₃) δ 8.10-8.14 (m, 4H), 7.72-7.79 (m, 2H), 7.48-7.56 (m, 4H), 7.42 (m, 1H), 7.20 (m, 1H); ¹³C NMR (100 MHz, CDCl₃) δ 163.2 (d, J=245.0 Hz), 150.5 (d, J=2.7 Hz), 137.8 (d, J=7.8 Hz), 133.0, 130.6 (d, J=8.2 Hz), 129.1, 128.8, 128.4, 128.1, 127.1, 126.9, 126.1, 122.7 (d, J=2.9 Hz), 117.0 (d, J=21.2 Hz), 115.3, 114.0 (d, J=23.2 Hz).

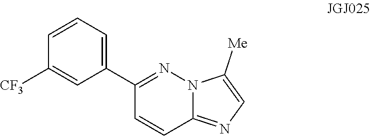 [SMILES: Cc1cnc2ccc(-c3cccc(C(F)(F)F)c3)nn12]

3-Methyl-6-(3-(trifluoromethyl)phenyl)imidazo[1,2-b]pyridazine, JGJ025. The reaction of 6-chloro-3-methylimidazolo[1,2-b]pyridazine (35.9 mg, 0.214 mmol), 3-trifluoromethylphenylboronic acid (42.7 mg, 0.225 mmol), K₂CO₃ (44.4 mg, 0.321 mmol) and Pd(PPh₃)₄ (12.4 mg, 0.011 mmol) in 1,4-dioxane/water (5:1 v/v, 0.4 mL) afforded the desired product JGJ018 (29.2 mg, 0.105 mmol, 49%) as a white solid using the same procedure as described for JGJ002. ¹H NMR (400 MHz, CDCl₃) δ 8.27 (s, 1H), 8.20 (d, J=8.0 Hz, 1H), 8.09 (d, J=9.2 Hz, 1H), 7.76 (d, J=8.0 Hz, 1H), 7.65-7.69 (m, 2H), 7.51 (d, J=9.2 Hz, 1H), 2.66 (s, 3H); ¹³C NMR (100 MHz, CDCl₃) δ 149.8, 136.7, 132.6, 132.1 (d, J=9.8 Hz), 131.5 (q, J=32.4 Hz), 130.2, 129.5, 128.4 (d, J=12.0 Hz), 126.4 (q, J=3.5 Hz), 125.7, 123.9 (q, J=270.8 Hz), 123.8 (q, J=3.8 Hz), 114.1, 8.7. (¹³C NMR will be taken again due to existence of some impurity)

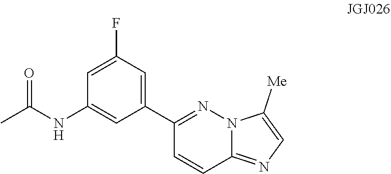 [SMILES: CC(=O)Nc1cc(F)cc(-c2ccc3ncc(C)n3n2)c1]

N-(3-Fluoro-5-(3-methylimidazo[1,2-b]pyridazin-6-yl)phenyl)acetamide, JGJ026. The reaction of 6-chloro-3-methylimidazo[1,2-b]pyridazine (35.3 mg, 0.211 mmol), 3-fluoro-5-aminophenylboronic acid (34.3 mg, 0.221 mmol), K₂CO₃ (43.7 mg, 0.316 mmol) and Pd(PPh₃)₄ (12.2 mg, 0.011 mmol) in 1,4-dioxane/water (5:1 v/v, 0.4 mL) afforded 3-fluoro-5-(3-methylimidazo[1,2-b]pyridazin-6-yl)aniline (25 mg, 0.103 mmol, 49%) as a pale yellow solid using the same procedure as described for JGJ002. Then the acetylation using the same procedure as described for JGJ004 gave the desired product JGJ026 (8 mg, 0.028 mmol, 28%) as a pale yellow solid. ¹H NMR (400 MHz, CDCl₃) δ 8.38 (br s, 1H), 8.00 (d, J=9.2 Hz, 1H), 7.89 (s, 1H), 7.65 (d, J=9.2 Hz, 1H), 7.60 (s, 1H), 7.43 (s, 1H), 7.41 (s, 1H), 2.60 (s, 3H), 2.24 (s, 3H);

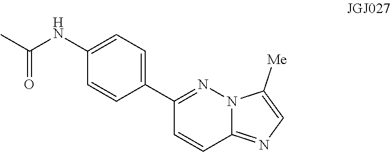 [SMILES: CC(=O)Nc1ccc(-c2ccc3ncc(C)n3n2)cc1]

N-(4-(3-Methylimidazo[1,2-b]pyridazin-6-yl)phenyl)acetamide, JGJ027. The reaction of 6-chloro-3-methyllimidazo[1,2-b]pyridazine (35.3 mg, 0.211 mmol), 4-aminophenylboronic acid (38.4 mg, 0.221 mmol), K₂CO₃ (43.7 mg, 0.316 mmol) and Pd(PPh₃)₄ (12.2 mg, 0.011 mmol) in 1,4-dioxane/water (5:1 v/v, 0.4 mL) afforded 4-(3-methylimidazo[1,2-b]pyridazin-6-yl)aniline (31.4 mg, 0.140 mmol, 66%) as a light yellow solid using the same procedure as described for JGJ002. Then the acetylation using the same procedure as described for JGJ004 gave the desired product JGJ026 (7.2 mg, 0.027 mmol, 19%) as an ivory solid. ¹H NMR (400 MHz, CDCl₃) δ 7.99 (d, J=8.8 Hz, 2H), 7.95 (d, J=9.2 Hz, 1H), 7.68 (d, J=8.4 Hz, 2H), 7.58 (s, 1H), 7.47 (br s, 1H), 7.43 (d, J=9.6 Hz, 1H), 2.62 (s, 3H), 2.23 (s, 3H);

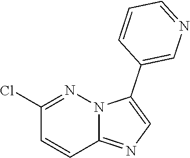 [SMILES: Clc1ccc2ncc(-c3cccnc3)n2n1]

6-Chloro-3-(pyridin-3-yl)imidazo[1,2-b]pyridazine. The reaction of 6-chloro-3-iodoimidazo[1,2-b]pyridazine (82.6 mg, 0.297 mmol), pyridine-3-boronic acid (40 mg, 0.325 mmol), K₂CO₃ (61.3 mg, 0.443 mmol) and Pd(PPh₃)₄ (17.1 mg, 0.015 mmol) in 1,4-dioxane/water (5:1 v/v, 1 mL) at 100° C. afforded the desired product (41.5 mg, 0.180, 61%) as a pale yellow solid using the same procedure as described for 6-chloro-3-phenylimidazo[1,2-b]pyridazine. ¹H NMR (400 MHz, CDCl₃) δ 9.21 (s, 1H), 8.62 (s, 1H), 8.40 (m, 1H), 8.11 (s, 1H), 7.98 (d, J=9.6 Hz, 1H), 7.43 (dd, J=7.6, 0.8 Hz, 1H), 7.12 (d, J=9.2 Hz, 1H); ¹³C NMR (100 MHz, CDCl₃) δ 149.0, 147.6, 147.2, 139.1, 133.6, 133.5, 127.4, 126.0, 124.3, 123.6, 118.9.

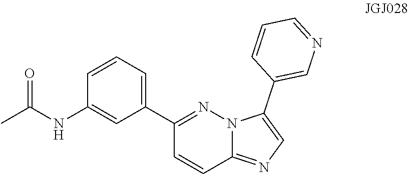 [SMILES: CC(=O)Nc1cccc(-c2ccc3ncc(-c4cccnc4)n3n2)c1]

N-(3-(3-(Pyridin-3-yl)imidazo[1,2-b]pyridazin-6-yl)phenyl)acetamide, JGJ028. The reaction of 6-chloro-3-(pyridin-3-yl)imidazo[1,2-b]pyridazine (41.5 mg, 0.180 mmol), 3-aminophenyl-boronic acid (30.7 mg, 0.198 mmol), K₂CO₃ (37.3 mg, 0.270 mmol) and Pd(PPh₃)₄ (10.4 mg, 0.009 mmol) in 1,4-dioxane/water (5:1 v/v, 0.4 mL) afforded 3-(3-(pyridin-3-yl)imidazo[1,2-b]pyridazin-6-yl)aniline (50.0 mg, 0.174 mmol, 96%) as an ivory solid using the same procedure as described for JGJ002. Then the acetylation using the same procedure as described for JGJ004 gave the desired product JGJ028 (18.2 mg, 0.055 mmol, 32%) as a pale yellow solid. ¹H NMR (400 MHz, CD₃OD) δ 9.29 (d, J=1.2 Hz, 1H), 8.60 (ddd, J=8.0, 2.0, 1.6 Hz, 1H), 8.49 (d, J=4.0 Hz, 1H), 8.25 (dd, J=2.0, 1.6 Hz, 1H), 8.18 (s, 1H), 8.02 (d, J=9.6 Hz, 1H), 7.68 (d, J=9.6 Hz, 1H), 7.60-7.65 (m, 2H), 7.55 (dd, J=8.0, 4.8 Hz, 1H), 7.37 (t, J=8.0 Hz, 1H), 2.16 (s, 3H); ¹³C NMR (100 MHz, CD₃OD) δ 172.1, 153.6, 149.2, 148.0, 141.5, 141.2, 137.1, 135.9, 134.1, 130.8, 127.1, 127.0, 126.9, 125.7, 123.8, 123.0, 119.5, 118.6, 24.3.

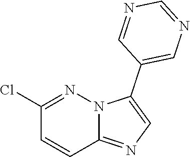 [SMILES: Clc1ccc2ncc(-c3cncnc3)n2n1]

6-Chloro-3-(pyrimidin-5-yl)imidazo[1,2-b]pyridazine. The reaction of 6-chloro-3-iodoimidazo[1,2-b]pyridazine (83.6 mg, 0.299 mmol), pyrimidine-5-boronic acid (40.8 mg, 0.329 mmol), K₂CO₃ (62 mg, 0.449 mmol) and Pd(PPh₃)₄ (17.3 mg, 0.015 mmol) in 1,4-dioxane/water (5:1 v/v, 1 mL) at 100° C. afforded the desired product (9.8 mg, 0.042 mmol, 14%) as a pale yellow solid using the same procedure as described for 6-chloro-3-phenylimidazo[1,2-b]pyridazine. ¹H NMR (400 MHz, CDCl₃) δ 9.42 (s, 2H), 9.23 (s, 1H), 8.18 (s, 1H), 8.04 (d, J=9.6 Hz, 1H), 7.20 (d, J=9.6 Hz, 1H); ¹³C NMR (100 MHz, CDCl₃) δ 157.7, 154.0, 147.7, 133.7, 132.1, 128.5, 127.7, 123.0, 119.8.

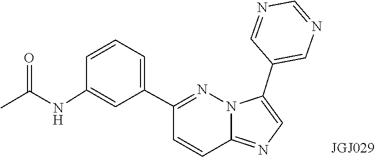 [SMILES: CC(=O)Nc1cccc(-c2ccc3ncc(-c4cncnc4)n3n2)c1]

N-(3-(3-(Pyrimidin-5-yl)imidazo[1,2-b]pyridazin-6-yl)phenyl)acetamide, JGJ029. The reaction of 6-chloro-3-(pyrimidin-5-yl)imidazo[1,2-b]pyridazine (9.8 mg, 0.042 mmol), 3-aminophenylboronic acid (7.2 mg, 0.047 mmol), K₂CO₃ (8.8 mg, 0.064 mmol) and Pd(PPh₃)₄ (4.9 mg, 0.004 mmol) in 1,4-dioxane/water (5:1 v/v, 0.2 mL) afforded 3-(3-(pyridin-3-yl)imidazo[1,2-b]pyridazin-6-yl)aniline (6.7 mg, 0.023 mmol, 55%) as a light yellow solid using the same procedure as described for JGJ002. Then the acetylation using the same procedure as described for JGJ004 gave the desired product JGJ029 (5.1 mg, 0.015 mmol, 67%) as an ivory solid. ¹H NMR (400 MHz, CDCl₃+5% v/v CD₃OD) δ 9.58 (s, 2H), 9.18 (s, 1H), 8.23 (s, 1H), 8.19 (d, J=9.6 Hz, 1H), 8.10 (s, 1H), 7.99 (d, J=8.0 Hz, 1H) 7.65 (d, J=9.2 Hz, 1H), 7.63 (d, J=8.0 Hz, 1H), 7.46 (dd, J=8.4, 7.6 Hz, 1H), 2.19 (s, 3H); ¹³C NMR (125 MHz, CDCl₃+5% v/v CD₃OD) δ 169.7, 156.8, 153.9, 152.5, 139.6, 134.7, 131.9, 129.9, 125.9, 123.7, 122.4, 122.2, 122.1, 118.0, 117.7, 117.6, 24.0.

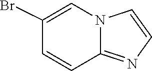

6-Bromoimidazo[1,2-a]pyridine. To a solution of 2-amino-5-bromopyridine (500 mg, 2.89 mmol) in EtOH (6 mL) and water (4 mL) was added bromoacetaldehyde diethyl acetal (870 μL, 5.78 mmol) and HBr (260 μL) at 23° C. The resulting mixture was heated at 103° C. overnight. After it was cooled to 23° C., the mixture was diluted in water and extracted with EtOAc. The combined organic layer was washed with saturated NaHCO₃ solution, dried over anhydrous MgSO₄, filtered and concentrated under reduced pressure. The resulting crude residue was used for the next step without further purification. (Brown solid; 331.7 mg, 1.68 mmol, 58%)¹H NMR (400 MHz, CDCl₃) δ 8.09 (dd, J=2.0, 0.8 Hz, 1H), 7.46 (d, J=0.8 Hz, 1H), 7.39 (s, 1H), 7.32 (d, J=9.6 Hz, 1H), 7.00 (dd, J=9.6, 2.0 Hz, 1H); ¹³C NMR (100 MHz, CDCl₃) δ 143.2, 133.8, 127.3, 125.4, 117.8, 112.3, 106.5.

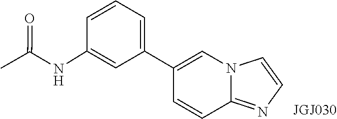 [SMILES: CC(=O)Nc1cccc(-c2ccc3nccn3c2)c1]

N-(3-(Imidazo[1,2-a]pyridin-6-yl)phenyl)acetamide, JGJ030. The reaction of 6-bromoimidazo[1,2-a]pyridine (50 mg, 0.254 mmol), 3-aminophenylboronic acid (43.3 mg, 0.279 mmol), K₂CO₃ (52.6 mg, 0.381 mmol) and Pd(PPh₃)₄ (29.3 mg, 0.025 mmol) in 1,4-dioxane/water (5:1 v/v, 1 mL) afforded 3-(imidazo[1,2-a]pyridin-6-yl)aniline (22.3 mg, 0.107 mmol, 42%) as an ivory solid using the same procedure as described for JGJ002. Then the acetylation using the same procedure as described for JGJ004 gave the desired product JGJ030 (13.6 mg, 0.054 mmol, 51%) as a white solid. ¹H NMR (400 MHz, CD₃OD) δ 8.68 (s, 1H), 7.89-7.94 (m, 2H), 7.56-7.62 (m, 3H), 7.51 (ddd, J=7.6, 2.0, 1.2 Hz, 1H), 7.35-7.43 (m, 2H), 2.16 (s, 3H); ¹³C NMR (100 MHz, CD₃OD) δ 170.3, 139.2, 137.4, 132.1, 129.1, 126.7, 125.7, 123.8, 122.1, 119.1, 118.0, 115.8, 113.5, 22.4. (one low-field carbon not observed)

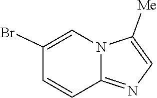 [SMILES: Cc1cnc2ccc(Br)cn12]

6-Bromo-3-methylimidazo[1,2-a]pyridine. A mixture of 2-amino-5-bromopyridine (200 mg, 1.156 mmol) and 2-bromopropionaldehyde (purity >95%, 318 mg, 2.312 mmol) in EtOH (5 mL) was heated at reflux overnight. After the mixture was cooled to 23° C., it was concentrated and extracted with EtOAc. The combined organic layer was washed with brine, dried over anhydrous MgSO₄, filtered and concentrated under reduced pressure. The crude residue was purified by flash column chromatography (n-Hexane:EtOAc=3:2) to obtain the desired product (86.9 mg, 0.412 mmol, 36%) as a white solid. ¹H NMR (400 MHz, CDCl₃) δ 8.00 (d, J=1.2 Hz, 1H), 7.49 (d, J=9.2 Hz, 1H), 7.40 (s, 1H), 7.20 (dd, J=9.6, 2.0 Hz, 1H), 2.46 (s, 3H); ¹³C NMR (100 MHz, CDCl₃) δ 143.5, 132.1, 126.5, 123.0, 120.3, 118.3, 106.9. 9.0.

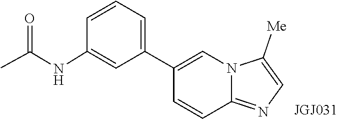 [SMILES: CC(=O)Nc1cccc(-c2ccc3ncc(C)n3c2)c1]

N-(3-(3-Methylimidazo[1,2-a]pyridin-6-yl)phenyl)acetamide, JGJ031. The reaction of 6-bromo-3-methylimidazo[1,2-a]pyridine (35 mg, 0.166 mmol), 3-aminophenylboronic acid (28.3 mg, 0.182 mmol), K₂CO₃ (34.4 mg, 0.249 mmol) and Pd(PPh₃)₄ (9.6 mg, 0.008 mmol) in 1,4-dioxane/water (5:1 v/v, 0.3 mL) afforded 3-(3-methylimidazo[1,2-a]pyridin-6-yl)aniline (28.1 mg, 0.106 mmol, 64%) as an ivory solid using the same procedure as described for JGJ002. Then the acetylation using the same procedure as described for JGJ004 gave the desired product JGJ031 (15.8 mg, 0.060 mmol, 56%) as an ivory solid. ¹H NMR (400 MHz, CDCl₃) δ 8.30 (br s, 1H), 8.12 (s, 1H), 7.87 (s, 1H), 7.65 (d, J=8.0 Hz, 1H), 7.54 (d, J=8.0 Hz, 1H), 7.36-7.43 (m, 3H), 7.27 (m, 1H), 2.49 (s, 3H), 2.23 (s, 3H);

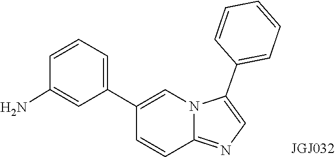 [SMILES: Nc1cccc(-c2ccc3ncc(-c4ccccc4)n3c2)c1]

3-(3-Phenylimidazo[1,2-a]pyridin-6-yl)aniline, JGJ032. To a mixture of 2-amino-5-bromo-pyridine (100 mg, 0.508 mmol), 3-aminophenylboronic acid (76.5 mg, 0.558 mmol), triphenylphosphine (26.6 mg, 0.102 mmol) and K₂CO₃ (140.3 mg, 1.015 mmol) in toluene: EtOH mixture (2:1 v/v, 1.7 mL) in a microwave tube was added Pd(OAc)₂ (11.4 mg, 0.059 mmol) and charged with argon. The mixture was sealed with a silicon septum and irradiated in microwave at 140° C. with stirring for 30 min. After the mixture had been allowed to cool to 23° C., bromobenzene (119.5 mg, 0.761 mmol) was injected into the tube by syringe and the mixture was again subjected to microwave irradiation at 140° C. with stirring for 2.5 h. The reaction vessel was cooled to 23° C. and the mixture was diluted with water and extracted with dichloromethane. The combined organic layer was dried over anhydrous MgSO₄, filtered and concentrated under reduced pressure. The crude residue was purified by flash column chromatography (n-Hexane:EtOAc: MeOH=1:1:0.1) to obtain the desired product (28.8 mg, 0.101 mmol, 20%) as a pale yellow solid. ¹H NMR (400 MHz, CDCl₃) δ 8.46 (s, 1H), 7.83 (d, J=9.2 Hz, 1H), 7.73 (s, 1H), 7.45-7.61 (m, 6H), 7.23 (d, J=8.0 Hz, 1H), 6.90 (d, J=8.0 Hz, 1H), 6.81 (t, J=2.0 Hz, 1H), 6.71 (m, 1H);

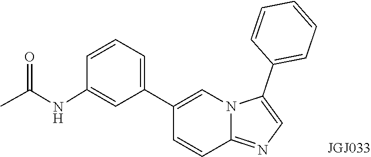 [SMILES: CC(=O)Nc1cccc(-c2ccc3ncc(-c4ccccc4)n3c2)c1]

N-(3-(3-Phenylimidazo[1,2-a]pyridin-6-yl)phenyl)acetamide, JGJ033. The reaction of 3-(3-phenylimidazo[1,2-a]pyridin-6-yl)aniline (JGJ032, 22.8 mg, 0.080 mmol), triethylamine (12.1 mg, 0.120 mmol) and acetyl chloride (9.4 mg, 0.120 mmol) in dichloromethane (2 mL) afforded the desired product JGJ033 (12.2 mg, 0.037 mmol, 47%) as an ivory solid using the same procedure as described for JGJ004. ¹H NMR (400 MHz, CD₃OD) δ 8.48 (s, 1H), 7.80 (dd, J=2.0, 1.6 Hz, 1H), 7.73 (s, 1H), 7.51-7.65 (m, 7H), 7.43 (m, 1H), 7.35 (dd, J=8.0 Hz, 1H), 7.27 (m, 1H), 2.12 (s, 3H); ¹³C NMR (100 MHz, CD₃OD) δ 170.3, 139.2, 137.4, 131.2, 129.2, 129.0, 128.4, 128.2, 127.7, 127.1, 126.5, 125.6, 122.0, 120.5, 119.0, 117.8, 116.5, 22.4. (one low-field carbon not observed)

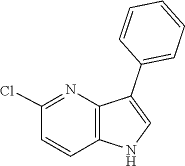 [SMILES: Clc1ccc2[nH]cc(-c3ccccc3)c2n1]

5-Chloro-3-phenyl-1H-pyrrolo[3,2-b]pyridine. To a solution of 2-chloro-5-hydrazinopyridine (71.3 mg, 0.5 mmol) in 4% w/w aqueous H₂SO₄ (5 mL) in a microwave tube was added (2,2-dimethoxyethyl)benzene (87.3 mg, 0.525 mmol). The reaction vessel was sealed with a silicon septum and stirred at 23° C. for 1 min then irradiated in microwave at 160° C. for 5 min. After the mixture was cooled to 23° C., it was slowly poured into 40% w/w KOH solution (5 mL). The mixture was extracted with EtOAc and the combined organic layer was dried over anhydrous MgSO₄, filtered and concentrated under reduced pressure. The resulting crude residue was purified by flash column chromatography (n-Hexane:EtOAc=3:2) to obtain the desired product (71.3 mg, 0.312 mmol, 62%) as a light yellow solid. ¹H NMR (400 MHz, CDCl₃) δ 8.96 (br s, 1H), 7.99 (d, J=7.2 Hz, 2H), 7.59 (s, 1H), 7.57 (d, J=8.8 Hz, 1H), 7.39 (t, J=7.6 Hz, 2H), 7.23 (dd, J=7.6, 7.2 Hz, 1H), 7.12 (d, J=8.9 Hz, 1H). The spectroscopic data match the literature data. [Ref: Eur. J. Org. Chem. 2013, 3328-3336.

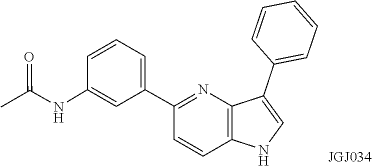 [SMILES: CC(=O)Nc1cccc(-c2ccc3[nH]cc(-c4ccccc4)c3n2)c1]

N-(3-(3-Phenyl-1H-pyrrolo[3,2-b]pyridin-5-yl)phenyl)acetamide, JGJ034. The reaction of 5-chloro-3-phenyl-1H-pyrrolo[3,2-b]pyridine (40 mg, 0.175 mmol), 3-aminophenylboronic acid (29.8 mg, 0.192 mmol), K₂CO₃ (36.3 mg, 0.262 mmol) and Pd(PPh₃)₄ (20.2 mg, 0.018 mmol) in 1,4-dioxane/water (5:1 v/v, 0.5 mL) afforded 3-(3-phenyl-1H-pyrrolo[3,2-b]pyridin-5-yl)aniline (18.8 mg, 0.066 mmol, 38%) as a white solid using the same procedure as described for JGJ002. Then the acetylation using the same procedure as described for JGJ004 gave the desired product JGJ034 (13.5 mg, 0.041 mmol, 63%) as an ivory solid. ¹H NMR (400 MHz, CD₃OD) δ 8.29 (s, 1H), 8.24 (d, J=7.2 Hz, 2H), 7.88 (s, 1H), 7.83 (d, J=7.6 Hz, 1H), 7.82 (d, J=8.8 Hz, 1H), 7.64 (d, J=8.4 Hz, 1H), 7.62 (d, J=7.6 Hz, 1H), 7.39-7.44 (m, 3H), 7.21 (dd, J=7.6, 7.2 Hz, 1H), 2.17 (s, 3H); ¹³C NMR (100 MHz, CD₃OD) δ 170.3, 150.1, 143.3, 141.1, 138.7, 134.5, 129.3, 128.5, 127.9, 126.3, 126.2, 125.1, 122.4, 119.3 (two peaks), 118.3, 115.7, 114.0, 22.4.

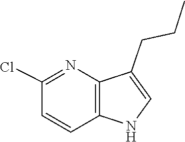

5-Chloro-3-propyl-1H-pyrrolo[3,2-b]pyridine. The reaction of 2-chloro-5-hydrazinopyridine (71.8 mg, 0.5 mmol) and valeraldehyde (45.1 mg, 0.524 mmol) in 4% w/w aq. H₂SO₄ (5 mL) afforded the desired product (56.7 mg, 0.291 mmol, 58%) as a pale yellow solid using the same procedure as described for 5-chloro-3-phenyl-1H-pyrrolo[3,2-b]pyridine. ¹H NMR (400 MHz, CDCl₃) δ 8.01 (br s, 1H), 7.61 (d, J=8.0 Hz, 1H), 7.26 (s, 1H), 7.08 (d, J=8.0 Hz, 1H), 2.77 (t, J=7.6 Hz, 2H), 1.73 (m, 2H), 0.94 (t, J=7.2 Hz, 3H); ¹³C NMR (100 MHz, CDCl₃) δ 145.0, 143.4, 127.8, 126.3, 120.9, 117.2, 116.6, 26.8, 23.0, 14.0.

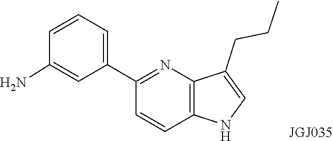 [SMILES: CCCc1c[nH]c2ccc(-c3cccc(N)c3)nc12]

3-(3-Propyl-1H-pyrrolo[3,2-b]pyridin-5-yl)aniline, JGJ035. The reaction of 5-chloro-3-propyl-1H-pyrrolo[3,2-b]pyridine (40 mg, 0.206 mmol), 3-aminophenylboronic acid (31 mg, 0.226 mmol), K₂CO₃ (42.6 mg, 0.308 mmol) and Pd(PPh₃)₄ (23.8 mg, 0.021 mmol) in 1,4-dioxane/water (5:1 v/v, 0.5 mL) afforded the desired product JGJ035 (42.5 mg, 0.169 mmol, 82%) as a white solid using the same procedure as described for JGJ002. ¹H NMR (400 MHz, CD₃OD) δ 7.72 (d, J=8.4 Hz, 1H), 7.44 (d, J=8.8 Hz, 1H), 7.34 (dd, J=2.0, 1.6 Hz, 1H), 7.30 (s, 1H), 7.24 (ddd, J=7.6, 1.6, 1.2 Hz, 1H), 7.19 (t, J=7.6 Hz, 1H), 6.76 (ddd, J=7.6, 2.0, 1.2 Hz, 1H), 2.85 (t, J=7.6 Hz, 2H), 1.79 (m, 2H), 1.02 (t, J=7.2 Hz, 3H); ¹³C NMR (100 MHz, CD₃OD) δ 152.4, 128.8, 146.2, 143.4, 130.2, 130.1, 127.6, 120.3, 118.8, 117.4, 116.3, 115.8, 27.1, 24.6, 14.5. (one low-field carbon not observed)

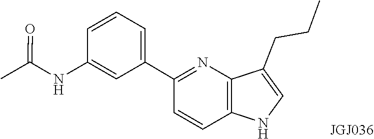 [SMILES: CCCc1c[nH]c2ccc(-c3cccc(NC(C)=O)c3)nc12]

N-(3-(3-Propyl-1H-pyrrolo[3,2-b]pyridin-5-yl)phenyl)acetamide, JGJ036. The reaction of 3-(3-propyl-1H-pyrrolo[3,2-b]pyridin-5-yl)aniline (JGJ035, 34.5 mg, 0.137 mmol), triethylamine (20.8 mg, 0.206 mmol) and acetyl chloride (16.2 mg, 0.206 mmol) in dichloromethane (3 mL) afforded the desired product JGJ036 (28.8 mg, 0.098 mmol, 72%) as an ivory solid using the same procedure as described for JGJ004. ¹H NMR (400 MHz, CD₃OD) δ 8.11 (dd, J=2.0, 1.6 Hz, 1H), 7.75 (d, J=8.4 Hz, 1H), 7.64-7.67 (m, 2H), 7.49 (d, J=8.8 Hz, 1H), 7.39 (t, J=8.0 Hz, 1H), 7.32 (s, 1H), 2.85 (t, J=7.2 Hz, 2H), 2.15 (s, 3H), 1.80 (m, 2H), 1.01 (t, J=7.2 Hz, 3H); ¹³C NMR (100 MHz, CD₃OD) δ 171.8, 151.4, 146.4, 143.1, 140.1, 130.3, 129.9, 127.9, 124.2, 120.6, 120.4, 120.2, 117.4, 115.7, 27.1, 24.5, 23.9, 14.5.

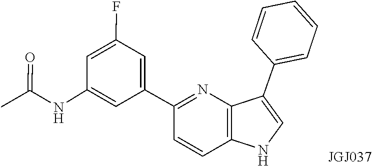 [SMILES: CC(=O)Nc1cc(F)cc(-c2ccc3[nH]cc(-c4ccccc4)c3n2)c1]

N-(3-Fluoro-5-(3-phenyl-1H-pyrrolo[3,2-b]pyridin-5-yl)phenyl)acetamide, JGJ037. The reaction of 5-chloro-3-phenyl-1H-pyrrolo[3,2-b]pyridine (19.4 mg, 0.085 mmol), 3-fluoro-5-aminophenylboronic acid (14.5 mg, 0.093 mmol), K₂CO₃ (17.6 mg, 0.127 mmol) and Pd(PPh₃)₄ (9.8 mg, 0.009 mmol) in 1,4-dioxane/water (5:1 v/v, 0.3 mL) afforded 3-fluoro-5-(3-phenyl-1H-pyrrolo[3,2-b]pyridin-5-yl)aniline (18.1 mg, 0.060 mmol, 70%) as an ivory solid using the same procedure as described for JGJ002. Then the acetylation using the same procedure as described for JGJ004 gave the desired product JGJ037 (13.8 mg, 0.040 mmol, 67%) as an ivory solid. ¹H NMR (400 MHz, CD₃OD) δ 8.25 (m, 2H), 7.98 (t, J=1.6 Hz, 1H), 7.88 (s, 1H), 7.79 (d, J=8.4 Hz, 1H), 7.61 (d, J=8.8 Hz, 1H), 7.58 (m, 2H), 7.43 (t, J=7.6 Hz, 2H), 7.21 (td, J=7.6, 1.2 Hz, 1H), 2.15 (s, 3H); ¹³C NMR (100 MHz, CD₃OD) δ 171.9, 164.6 (d, J=239.6 Hz), 150.2 (d, J=2.9 Hz), 145.1, 144.7 (d, J=8.9 Hz), 141.7 (d, J=11.5 Hz), 136.0, 130.9, 129.4, 127.8, 127.6, 126.6, 120.5, 117.3, 115.3, 114.7 (d, J=3.2 Hz), 109.8 (d, J=23.1 Hz), 107.3 (d, J=27.0 Hz), 24.0.

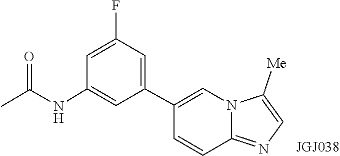 [SMILES: CC(=O)Nc1cc(F)cc(-c2ccc3ncc(C)n3c2)c1]

N-(3-Fluoro-5-(3-methylimidazo[1,2-a]pyridin-6-yl)phenyl)acetamide, JGJ038. The reaction of 6-bromo-3-methylimidazo[1,2-a]pyridine (23.4 mg, 0.111 mmol), 3-fluoro-5-aminophenyl-boronic acid (18.9 mg, 0.122 mmol), K₂CO₃ (23.0 mg, 0.166 mmol) and Pd(PPh₃)₄ (12.8 mg, 0.011 mmol) in 1,4-dioxane/water (5:1 v/v, 0.3 mL) afforded 3-fluoro-5-(3-methylimidazo[1,2-a]pyridin-6-yl)aniline (13.2 mg, 0.055 mmol, 49%) as a pale yellow solid using the same procedure as described for JGJ002. Then the acetylation using the same procedure as described for JGJ004 gave the desired product JGJ038 (8.3 mg, 0.029 mmol, 64%) as an ivory solid. ¹H NMR (400 MHz, CD₃OD) δ 8.41 (s, 1H), 7.56-7.63 (m, 3H), 7.52 (dt, J=10.8, 2.0 Hz, 1H), 7.40 (s, 1H), 7.23 (dt, J=9.6, 2.0 Hz, 1H), 2.57 (s, 3H), 2.17 (s, 3H);

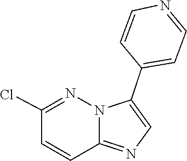 [SMILES: Clc1ccc2ncc(-c3ccncc3)n2n1]

6-Chloro-3-(pyridin-4-yl)imidazo[1,2-b]pyridazine. The reaction of 6-chloro-3-iodoimidazo[1,2-b]pyridazine (90.5 mg, 0.324 mmol), 4-pyridineboronic acid (43.8 mg, 0.356 mmol), K₂CO₃ (67.1 mg, 0.486 mmol) and Pd(PPh₃)₄ (37.4 mg, 0.032 mmol) in 1,4-dioxane/water (5:1 v/v, 0.7 mL) at 100° C. afforded the desired product (15.3 mg, 0.066 mmol, 20%) as a pale yellow solid using the same procedure as described for 6-chloro-3-phenylimidazo[1,2-b]pyridazine. ¹H NMR (400 MHz, CDCl₃) δ 8.72 (d, J=5.2 Hz, 2H), 8.23 (s, 1H), 7.98-8.02 (m, 3H), 7.18 (d, J=9.2 Hz, 1H); ¹³C NMR (100 MHz, CDCl₃) δ 150.2, 147.3, 139.8, 135.2, 134.9, 127.5, 126.1, 119.9, 119.4.

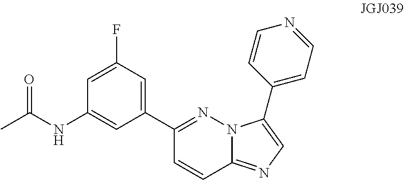 [SMILES: CC(=O)Nc1cc(F)cc(-c2ccc3ncc(-c4ccncc4)n3n2)c1]

N-(3-Fluoro-5-(3-(pyridin-4-yl)imidazo[1,2-b]pyridazin-6-yl)phenyl)acetamide, JGJ039. The reaction of 6-chloro-3-(pyridin-4-yl)imidazo[1,2-b]pyridazine (15.3 mg, 0.066 mmol), 3-fluoro-5-aminophenylboronic acid (11.3 mg, 0.073 mmol), K₂CO₃ (13.7 mg, 0.100 mmol) and Pd(PPh₃)₄ (7.7 mg, 0.007 mmol) in 1,4-dioxane/water (5:1 v/v, 0.3 mL) afforded 3-fluoro-5-(3-(pyridin-4-yl)imidazo[1,2-b]pyridazin-6-yl)aniline (10.7 mg, 0.035 mmol, 53%) as a light yellow solid using the same procedure as described for JGJ002. Then the acetylation using the same procedure as described for JGJ004 gave the desired product JGJ039 (3.8 mg, 0.011 mmol, 31%) as a pale yellow solid. ¹H NMR (400 MHz, CD₃OD) δ 8.69 (s, 2H), 8.46 (s, 1H), 8.36 (d, J=5.2 Hz, 2H), 8.20-8.23 (m, 2H), 7.88 (d, J=9.2 Hz, 1H), 7.64 (dt, J=10.8, 1.6 Hz, 1H), 7.55 (dt, 9.6, 1.6 Hz, 1H), 2.20 (s, 3H).

### REFERENCES

1. Dohner H, Weisdorf D J, Bloomfield C D. Acute Myeloid Leukemia. The New England journal of medicine. 2015; 373:1136-52.
2. Jordan C T. Unique molecular and cellular features of acute myelogenous leukemia stem cells. Leukemia. 2002; 16:559-62.
3. Dick J E. Acute myeloid leukemia stem cells. Annals of the New York Academy of Sciences. 2005; 1044:1-5.
4. Rosen J M, Jordan C T. The increasing complexity of the cancer stem cell paradigm. Science (New York, N.Y.). 2009; 324:1670-3.
5. Ambros V. microRNAs: tiny regulators with great potential. Cell. 2001; 107:823-6.
6. Bartel D P. MicroRNAs: genomics, biogenesis, mechanism, and function. Cell. 2004; 116:281-97.
7. Ambros V. The functions of animal microRNAs. Nature. 2004; 431:350-5.
8. Calin G A, Croce C M. MicroRNA signatures in human cancers. Nature reviews Cancer. 2006; 6:857-66.
9. Calin G A, Croce C M. MicroRNA-cancer connection: the beginning of a new tale. Cancer research. 2006; 66:7390-4.
10. Jongen-Lavrencic M, Sun S M, Dijkstra M K, Valk P J M, Lowenberg B. MicroRNA expression profiling in relation to the genetic heterogeneity of acute myeloid leukemia. Blood. 2008; 111:5078-85.
11. Garzon R, Volinia S, Liu C G, Fernandez-Cymering C, Palumbo T, Pichiorri F, Fabbri M, Coombes K, Alder H, Nakamura T, Flomenberg N, Marcucci G, Calin G A, Kornblau S M, Kantarjian H, Bloomfield C D, Andreeff M, Croce C M. MicroRNA signatures associated with cytogenetics and prognosis in acute myeloid leukemia. Blood. 2008; 111(6):3183-9. Epub 2008/01/12. doi: 10.1182/blood-2007-07-098749. PubMed PMID: 18187662; PMCID: PMC2265455.
12. Chen L, Sun Y, Wang J, Jiang H, Muntean A G. Differential regulation of the c-Myc/Lin28 axis discriminates subclasses of rearranged MLL leukemia. Oncotarget. 2016; 7(18):25208-23. Epub 2016/03/24. doi: 10.18632/oncotarget.8199. PubMed PMID: 27007052; PMCID: PMC5041898.
13. Nair V S, Maeda L S, Ioannidis J P A. Clinical outcome prediction by microRNAs in human cancer: a systematic review. Journal of the National Cancer Institute. 2012; 104:528-40.
14. Dixon-McIver A, East P, Mein C A, Cazier J-B, Molloy G, Chaplin T, Andrew Lister T, Young B D, Debernardi S. Distinctive patterns of microRNA expression associated with karyotype in acute myeloid leukaemia. PloS one. 2008; 3:e2141.
15. Johnson C D, Esquela-Kerscher A, Stefani G, Byrom M, Kelnar K, Ovcharenko D, Wilson M, Wang X, Shelton J, Shingara J, Chin L, Brown D, Slack F J. The let-7 microRNA represses cell proliferation pathways in human cells. Cancer Res. 2007; 67(16):7713-22. Epub 2007/08/19. doi: 10.1158/0008-5472.CAN-07-1083. PubMed PMID: 17699775.
16. Roush S, Slack F J. The let-7 family of microRNAs. Trends in cell biology. 2008; 18:505-16.
17. Viswanathan S R, Daley G Q, Gregory R I. Selective blockade of microRNA processing by Lin28. Science (New York, N.Y.). 2008; 320:97-100.
18. Newman M A, Thomson J M, Hammond S M. Lin-28 interaction with the Let-7 precursor loop mediates regulated microRNA processing. RNA (New York, N.Y.). 2008; 14:1539-49.
19. Heo I, Joo C, Cho J, Ha M, Han J, Kim V N. Lin28 mediates the terminal uridylation of let-7 precursor MicroRNA. Molecular cell. 2008; 32:276-84.
20. Piskounova E, Viswanathan S R, Janas M, LaPierre R J, Daley G Q, Sliz P, Gregory R I. Determinants of microRNA processing inhibition by the developmentally regulated RNA-binding protein Lin28. The Journal of biological chemistry. 2008; 283:21310-4.
21. Rybak A, Fuchs H, Smirnova L, Brandt C, Pohl E E, Nitsch R, Wulczyn F G. A feedback loop comprising lin-28 and let-7 controls pre-let-7 maturation during neural stem-cell commitment. Nature cell biology. 2008; 10:987-93.
22. Viswanathan S R, Powers J T, Einhorn W, Hoshida Y, Ng T L, Toffanin S, O'Sullivan M, Lu J, Phillips L A, Lockhart V L, Shah S P, Tanwar P S, Mermel C H, Beroukhim R, Azam M, Teixeira J, Meyerson M, Hughes T P, Llovet J M, Radich J, Mullighan C G, Golub T R, Sorensen P H, Daley G Q. Lin28 promotes transformation and is associated with advanced human malignancies. Nature genetics. 2009; 41:843-8.
23. Iliopoulos D, Hirsch H A, Struhl K. An epigenetic switch involving NF-kappaB, Lin28, Let-7 MicroRNA, and IL6 links inflammation to cell transformation. Cell. 2009; 139:693-706.
24. Albino D, Civenni G, Dallavalle C, Roos M, Jahns H, Curti L, Rossi S, Pinton S, D'Ambrosio G, Sessa F, Hall J, Catapano C V, Carbone G M. Activation of the Lin28/let-7 Axis by Loss of ESE3/EHF Promotes a Tumorigenic and Stem-like Phenotype in Prostate Cancer. Cancer research. 2016; 76:3629-43.
25. King C E, Cuatrecasas M, Castells A, Sepulveda A R, Lee J-S, Rustgi A K. LIN28B promotes colon cancer progression and metastasis. Cancer research. 2011; 71:4260-8.
26. Zhang W C, Shyh-Chang N, Yang H, Rai A, Umashankar S, Ma S, Soh B S, Sun L L, Tai B C, Nga M E, Bhakoo K K, Jayapal S R, Nichane M, Yu Q, Ahmed D A, Tan C, Sing W P, Tam J, Thirugananam A, Noghabi M S, Pang Y H, Ang H S, Mitchell W, Robson P, Kaldis P, Soo R A, Swarup S, Lim E H, Lim B. Glycine decarboxylase activity drives non-small cell lung cancer tumor-initiating cells and tumorigenesis. Cell. 2012; 148:259-72.
27. Kong D, Banerjee S, Ahmad A, Li Y, Wang Z, Sethi S, Sarkar F H. Epithelial to mesenchymal transition is mechanistically linked with stem cell signatures in prostate cancer cells. PloS one. 2010; 5:e12445.
28. Nam Y, Chen C, Gregory R I, Chou J J, Sliz P. Molecular basis for interaction of let-7 microRNAs with Lin28. Cell. 2011; 147(5):1080-91. Epub 2011/11/15. doi: 10.1016/j.cell.2011.10.020. PubMed PMID: 22078496; PMCID: PMC3277843.
29. Loughlin F E, Gebert L F, Towbin H, Brunschweiger A, Hall J, Allain F H. Structural basis of pre-let-7 miRNA recognition by the zinc knuckles of pluripotency factor Lin28. Nat Struct Mol Biol. 2011; 19(1):84-9. Epub 2011/12/14. doi: 10.1038/nsmb.2202. PubMed PMID: 22157959.
30. Wang L, Nam Y, Lee A K, Yu C, Roth K, Chen C, Ransey E M, Sliz P. LIN28 Zinc Knuckle Domain Is Required and Sufficient to Induce let-7 Oligouridylation. Cell Rep. 2017; 18(11):2664-75. Epub 2017/03/16. doi: 10.1016/j.celrep.2017.02.044. PubMed PMID: 28297670.
31. Hagan J P, Piskounova E, Gregory R I. Lin28 recruits the TUTase Zcchc11 to inhibit let-7 maturation in mouse embryonic stem cells. Nat Struct Mol Biol. 2009; 16(10):1021-5. Epub 2009/08/29. doi: 10.1038/nsmb.1676. PubMed PMID: 19713958; PMCID: PMC2758923.
32. Yan B X, Ma J X, Zhang J, Guo Y, Riedel H, Mueller M D, Remick S C, Yu J J. PSP94 contributes to chemoresistance and its peptide derivative PCK3145 represses tumor growth in ovarian cancer. Oncogene. 2014; 33(45):5288-94. Epub 2013/11/05. doi: 10.1038/onc.2013.466. PubMed PMID: 24186202.
33. Wang T, Han P, He Y, Zhao C, Wang G, Yang W, Shan M, Zhu Y, Yang C, Weng M, Wu D, Gao L, Jin X, Wei Y, Cui B, Shen G, Li X. Lin28A enhances chemosensitivity of colon cancer cells to 5-FU by promoting apoptosis in a let-7 independent manner. Tumour Biol. 2016; 37(6):7657-65. Epub 2015/12/22. doi: 10.1007/s13277-015-4559-8. PubMed PMID: 26687759.
34. Teng R, Hu Y, Zhou J, Seifer B, Chen Y, Shen J, Wang L. Overexpression of Lin28 Decreases the Chemosensitivity of Gastric Cancer Cells to Oxaliplatin, Paclitaxel, Doxorubicin, and Fluorouracil in Part via microRNA-107. PLoS One. 2015; 10(12):e0143716. Epub 2015/12/05. doi: 10.1371/journal.pone.0143716. PubMed PMID: 26636340; PMCID: PMC4670127.
35. Chaudhry M A, Sachdeva H, Omaruddin R A. Radiation-induced micro-RNA modulation in glioblastoma cells differing in DNA-repair pathways. DNA Cell Biol. 2010; 29(9):553-61. Epub 2010/04/13. doi: 10.1089/dna.2009.0978. PubMed PMID: 20380575.
36. Yang X, Cai H, Liang Y, Chen L, Wang X, Si R, Qu K, Jiang Z, Ma B, Miao C, Li J, Wang B, Gao P. Inhibition of c-Myc by let-7b mimic reverses mutidrug resistance in gastric cancer cells. Oncol Rep. 2015; 33(4):1723-30. Epub 2015/01/31. doi: 10.3892/or.2015.3757. PubMed PMID: 25633261.
37. Boyerinas B, Park S M, Murmann A E, Gwin K, Montag A G, Zillhardt M, Hua Y J, Lengyel E, Peter M E. Let-7 modulates acquired resistance of ovarian cancer to Taxanes via IMP-1-mediated stabilization of multidrug resistance 1. Int J Cancer. 2012; 130(8):1787-97. Epub 2011/05/28. doi: 10.1002/ijc.26190. PubMed PMID: 21618519; PMCID: PMC3230767.
38. Zhou J, Chan Z-L, Bi C, Lu X, Chong P S Y, Chooi J-Y, Cheong L-L, Liu S-C, Ching Y Q, Zhou Y, Osato M, Tan T Z, Ng C H, Ng S-B, Wang S, Zeng Q, Chng W-J. LIN28B Activation by PRL-3 Promotes Leukemogenesis and a Stem Cell-like Transcriptional Program in AML. Molecular cancer research: MCR. 2017; 15:294-303.
39. Chen Y, Jacamo R, Konopleva M, Garzon R, Croce C, Andreeff M. CXCR4 downregulation of let-7a drives chemoresistance in acute myeloid leukemia. J Clin Invest. 2013; 123(6):2395-407. Epub 2013/05/17. doi: 10.1172/JC166553. PubMed PMID: 23676502; PMCID: PMC3668829.
40. Campos M, Llorens C, Sempere J M, Futami R, Rodriguez I, Carrasco P, Capilla R, Latorre A, Coque T M, Moya A, Baquero F. A membrane computing simulator of trans-hierarchical antibiotic resistance evolution dynamics in nested ecological compartments (ARES). Biol Direct. 2015; 10:41. Epub 2015/08/06. doi: 10.1186/s13062-015-0070-9. PubMed PMID: 26243297; PMCID: PMC4526193.
41. Mehta S V, Shukla S N, Vora H H. Overexpression of Bcl2 protein predicts chemoresistance in acute myeloid leukemia: its correlation with FLT3. Neoplasma. 2013; 60(6):666-75. Epub 2013/08/03. doi: 10.4149/neo_2013_085. PubMed PMID: 23906301.
42. Fennell D A, Corbo M V, Dean N M, Monia B P, Cotter F E. In vivo suppression of Bcl-XL expression facilitates chemotherapy-induced leukaemia cell death in a SCID/NOD-Hu model. Br J Haematol. 2001; 112(3):706-13. Epub 2001/03/22. PubMed PMID: 11260076.
43. Lagadinou E D, Sach A, Callahan K, Rossi R M, Neering S J, Minhajuddin M, Ashton J M, Pei S, Grose V, O'Dwyer K M, Liesveld J L, Brookes P S, Becker M W, Jordan C T. BCL-2 inhibition targets oxidative phosphorylation and selectively eradicates quiescent human leukemia stem cells. Cell Stem Cell. 2013; 12(3):329-41. Epub 2013/01/22. doi: 10.1016/j.stem.2012.12.013. PubMed PMID: 23333149; PMCID: PMC3595363.
44. Agarwal V, Bell G W, Nam J W, Bartel D P. Predicting effective microRNA target sites in mammalian mRNAs. Elife. 2015; 4. Epub 2015/08/13. doi: 10.7554/eLife.05005. PubMed PMID: 26267216; PMCID: PMC4532895.
45. Guzman M L, Neering S J, Upchurch D, Grimes B, Howard D S, Rizzieri D A, Luger S M, Jordan C T. Nuclear factor-kappaB is constitutively activated in primitive human acute myelogenous leukemia cells. Blood. 2001; 98(8):2301-7. Epub 2001/10/06. PubMed PMID: 11588023.
46. Kagoya Y, Yoshimi A, Kataoka K, Nakagawa M, Kumano K, Arai S, Kobayashi H, Saito T, Iwakura Y, Kurokawa M. Positive feedback between NF-kappaB and TNF-alpha promotes leukemia-initiating cell capacity. J Clin Invest. 2014; 124(2):528-42. Epub 2014/01/03. doi: 10.1172/JC168101. PubMed PMID: 24382349; PMCID: PMC3904603.
47. Shinoda G, Shyh-Chang N, Soysa TYd, Zhu H, Seligson M T, Shah S P, Abo-Sido N, Yabuuchi A, Hagan J P, Gregory R I, Asara J M, Cantley L C, Moss E G, Daley G Q. Fetal deficiency of lin28 programs life-long aberrations in growth and glucose metabolism. Stem cells (Dayton, Ohio). 2013; 31:1563-73.
48. Yuan J, Nguyen C K, Liu X, Kanellopoulou C, Muljo S A. Lin28b reprograms adult bone marrow hematopoietic progenitors to mediate fetal-like lymphopoiesis. Science. 2012; 335(6073):1195-200. Epub 2012/02/22. doi: 10.1126/science.1216557. PubMed PMID: 22345399; PMCID: PMC3471381.
49. Copley M R, Babovic S, Benz C, Knapp DJHF, Beer P A, Kent D G, Wohrer S, Treloar D Q, Day C, Rowe K, Mader H, Kuchenbauer F, Humphries R K, Eaves C J. The Lin28b-let-7-Hmga2 axis determines the higher self-renewal potential of fetal haematopoietic stem cells. Nature cell biology. 2013; 15:916-25.
50. Rowe R G, Wang L D, Coma S, Han A, Mathieu R, Pearson D S, Ross S, Sousa P, Nguyen P T, Rodriguez A, Wagers A J, Daley G Q. Developmental regulation of myeloerythroid progenitor function by the Lin28b-let-7-Hmga2 axis. J Exp Med. 2016; 213(8):1497-512. Epub 2016/07/13. doi: 10.1084/jem.20151912. PubMed PMID: 27401346; PMCID: PMC4986532.
51. Roos M, Pradere U, Ngondo R P, Behera A, Allegrini S, Civenni G, Zagalak J A, Marchand J-R, Menzi M, Towbin H, Scheuermann J, Neri D, Caflisch A, Catapano C V, Ciaudo C, Hall J. A Small-Molecule Inhibitor of Lin28. ACS chemical biology. 2016; 11:2773-81.
52. Lim D, Byun W G, Koo J Y, Park H, Park S B. Discovery of a Small-Molecule Inhibitor of Protein-MicroRNA Interaction Using Binding Assay with a Site-Specifically Labeled Lin28. J Am Chem Soc. 2016. Epub 2016/09/27. doi: 10.1021/jacs.6b06965. PubMed PMID: 27668966.
53. Wang L, Rowe R G, Jaimes A, Yu C, Nam Y, Pearson D S, Zhang J, Xie X, Marion W, Heffron G J, Daley G Q, Sliz P. Small-Molecule Inhibitors Disrupt let-7 Oligouridylation and Release the Selective Blockade of let-7 Processing by LIN28. Cell Rep. 2018; 23(10):3091-101. Epub 2018/06/07. doi: 10.1016/j.celrep.2018.04.116. PubMed PMID: 29874593.
54. Balzeau J, Menezes M R, Cao S, Hagan J P. The LIN28/let-7 Pathway in Cancer. Front Genet. 2017; 8:31. Epub 2017/04/13. doi: 10.3389/fgene.2017.00031. PubMed PMID: 28400788; PMCID: PMC5368188.
55. Bagger F O, Kinalis S, Rapin N. BloodSpot: a database of healthy and malignant haematopoiesis updated with purified and single cell mRNA sequencing profiles. Nucleic Acids Res. 2019; 47(D1):D881-D5. Epub 2018/11/06. doi: 10.1093/nar/gky1076. PubMed PMID: 30395307; PMCID: PMC6323996.
56. Jiang X, Huang H, Li Z, Li Y, Wang X, Gurbuxani S, Chen P, He C, You D, Zhang S, Wang J, Arnovitz S, Elkahloun A, Price C, Hong G-M, Ren H, Kunjamma R B, Neilly M B, Matthews J M, Xu M, Larson R A, Le Beau M M, Slany R K, Liu P P, Lu J, Zhang J, He C, Chen J. Blockade of miR-150 maturation by MLL-fusion/MYC/LIN-28 is required for MLL-associated leukemia. Cancer cell. 2012; 22:524-35.
57. Stavropoulou V, Kaspar S, Brault L, Sanders M A, Juge S, Morettini S, Tzankov A, Iacovino M, Lau I-J, Milne T A, Royo H, Kyba M, Valk P J M, Peters A H F M, Schwaller J. MLL-AF9 Expression in Hematopoietic Stem Cells Drives a Highly Invasive AML Expressing EMT-Related Genes Linked to Poor Outcome. Cancer cell. 2016; 30:43-58.
58. Pang M, Wu G, Hou X, Hou N, Liang L, Jia G, Shuai P, Luo B, Wang K, Li G. LIN28B promotes colon cancer migration and recurrence. PLoS One. 2014; 9(10):e109169. Epub 2014/11/02. doi: 10.1371/journal.pone.0109169. PubMed PMID: 25360631; PMCID: PMC4215835.
59. Dai Y, Guzman M L, Chen S, Wang L, Yeung S K, Pei X Y, Dent P, Jordan C T, Grant S. The NF (Nuclear factor)-kappaB inhibitor parthenolide interacts with histone deacetylase inhibitors to induce MKK7/JNK1-dependent apoptosis in human acute myeloid leukaemia cells. Br J Haematol. 2010; 151(1):70-83. Epub 2010/08/13. doi: 10.1111/j.1365-2141.2010.08319.x. PubMed PMID: 20701602; PMCID: PMC2950247.
60. Guzman M L, Rossi R M, Karnischky L, Li X, Peterson D R, Howard D S, Jordan C T. The sesquiterpene lactone parthenolide induces apoptosis of human acute myelogenous leukemia stem and progenitor cells. Blood. 2005; 105(11):4163-9. Epub 2005/02/03. doi: 10.1182/blood-2004-10-4135. PubMed PMID: 15687234; PMCID: PMC1895029.
61. Gal H, Amariglio N, Trakhtenbrot L, Jacob-Hirsh J, Margalit O, Avigdor A, Nagler A, Tavor S, Ein-Dor L, Lapidot T, Domany E, Rechavi G, Givol D. Gene expression profiles of AML derived stem cells; similarity to hematopoietic stem cells. Leukemia. 2006; 20(12):2147-54. Epub 2006/10/14. doi: 10.1038/sj.leu.2404401. PubMed PMID: 17039238.
62. Yagi T, Morimoto A, Eguchi M, Hibi S, Sako M, Ishii E, Mizutani S, Imashuku S, Ohki M, Ichikawa H. Identification of a gene expression signature associated with pediatric AML prognosis. Blood. 2003; 102(5):1849-56. Epub 2003/05/10. doi: 10.1182/blood-2003-02-0578. PubMed PMID: 12738660.
63. Santos, R. et al. A comprehensive map of molecular drug targets. Nat Rev Drug Discov 16, 19-34, doi:10.1038/nrd.2016.230 (2017).
64. Barouch-Bentov, R. & Sauer, K. Mechanisms of drug resistance in kinases. Expert Opin Investig Drugs 20, 153-208, doi:10.1517/13543784.2011.546344 (2011).
65. Warner, K. D., Hajdin, C. E. & Weeks, K. M. Principles for targeting RNA with drug-like small molecules. Nat Rev Drug Discov 17, 547-558, doi:10.1038/nrd.2018.93 (2018).
66. Connelly, C. M., Moon, M. H. & Schneekloth, J. S., Jr. The Emerging Role of RNA as a Therapeutic Target for Small Molecules. Cell Chem Biol 23, 1077-1090, doi:10.1016/j.chembiol.2016.05.021 (2016).
67. Hentze, M. W., Castello, A., Schwarzl, T. & Preiss, T. A brave new world of RNA-binding proteins. Nat Rev Mol Cell Biol 19, 327-341, doi:10.1038/nrm.2017.130 (2018).
68. Elcheva, I. A. & Spiegelman, V. S. Targeting RNA-binding proteins in acute and chronic leukemia. Leukemia, doi:10.1038/s41375-020-01066-4 (2020).
69. Polesskaya, A. et al. Lin-28 binds IGF-2 mRNA and participates in skeletal myogenesis by increasing translation efficiency. Genes Dev 21, 1125-1138, doi:10.1101/gad.415007 (2007).
70. Hafner, M. et al. Transcriptome-wide identification of RNA-binding protein and microRNA target sites by PAR-CLIP. Cell 141, 129-141, doi:10.1016/j.cell.2010.03.009 (2010).
71. Liberzon, A. et al. The Molecular Signatures Database (MSigDB) hallmark gene set collection. Cell Syst 1, 417-425, doi:10.1016/j.cels.2015.12.004 (2015).
72. Keskin, T. et al. LIN28B Underlies the Pathogenesis of a Subclass of Ewing Sarcoma. Cell Rep 31, 107539, doi:10.1016/j.celrep.2020.107539 (2020).
73. Chen, C. et al. Targeting LIN28B reprograms tumor glucose metabolism and acidic microenvironment to suppress cancer stemness and metastasis. Oncogene 38, 4527-4539, doi:10.1038/s41388-019-0735-4 (2019).
74. Zuber, J. et al. Mouse models of human AML accurately predict chemotherapy response. Genes Dev 23, 877-889, doi:10.1101/gad.1771409 (2009).
75. Huber, K. V. M. et al. Proteome-wide drug and metabolite interaction mapping by thermal-stability profiling. Nature methods 12, 1055-1057 (2015).
76. Kwon, S. C. et al. The RNA-binding protein repertoire of embryonic stem cells. Nat Struct Mol Biol 20, 1122-1130, doi:10.1038/nsmb.2638 (2013).
77. Liu, Z., Scannell, D. R., Eisen, M. B. & Tjian, R. Control of embryonic stem cell lineage commitment by core promoter factor, TAF3. Cell 146, 720-731, doi:10.1016/j.cell.2011.08.005 (2011).
78. Liu, S. et al. Binding of the human Prp31 Nop domain to a composite RNA-protein platform in U4 snRNP. Science 316, 115-120, doi:10.1126/science.1137924 (2007).
79. Adler, A. S. et al. An integrative analysis of colon cancer identifies an essential function for PRPF6 in tumor growth. Genes Dev 28, 1068-1084, doi:10.1101/gad.237206.113 (2014).
80. Chan, S. et al. Basal-A Triple-Negative Breast Cancer Cells Selectively Rely on RNA Splicing for Survival. Mol Cancer Ther 16, 2849-2861, doi:10.1158/1535-7163.MCT-17-0461 (2017).
81. Dvinge, H., Guenthoer, J., Porter, P. L. & Bradley, R. K. RNA components of the spliceosome regulate tissue- and cancer-specific alternative splicing. Genome Res 29, 1591-1604, doi:10.1101/gr.246678.118 (2019).
82. Park, S. et al. PRPF4 is a novel therapeutic target for the treatment of breast cancer by influencing growth, migration, invasion, and apoptosis of breast cancer cells via p38 MAPK signaling pathway. Mol Cell Probes 47, 101440, doi:10.1016/j.mcp.2019.101440 (2019).
83. Song, H. et al. Splicing factor PRPF6 upregulates oncogenic androgen receptor signaling pathway in hepatocellular carcinoma. Cancer Sci, doi:10.1111/cas.14595 (2020).
84. Wang, E. T. et al. Alternative isoform regulation in human tissue transcriptomes. Nature 456, 470-476, doi:10.1038/nature07509 (2008).
85. Islam, S. U., Shehzad, A., Sonn, J. K. & Lee, Y. S. PRPF overexpression induces drug resistance through actin cytoskeleton rearrangement and epithelial-mesenchymal transition. Oncotarget 8, 56659-56671, doi:10.18632/oncotarget.17855 (2017).
86. Wang, E. & Aifantis, I. RNA Splicing and Cancer. Trends Cancer 6, 631-644, doi:10.1016/j.trecan.2020.04.011 (2020).
87. Berg, K. C. G. et al. Multi-omics of 34 colorectal cancer cell lines—a resource for biomedical studies. Mol Cancer 16, 116, doi:10.1186/s12943-017-0691-y (2017).
88. Augenlicht, L. H. et al. Low-level c-myc amplification in human colonic carcinoma cell lines and tumors: a frequent, p53-independent mutation associated with improved outcome in a randomized multi-institutional trial. Cancer Res 57, 1769-1775 (1997).
89. Cinkornpumin, J. et al. A small molecule screen to identify regulators of let-7 targets. Sci Rep 7, 15973, doi:10.1038/s41598-017-16258-9 (2017)

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of formula (I): 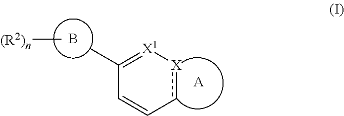
or a pharmaceutically acceptable salt thereof, wherein: 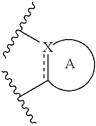
is selected from 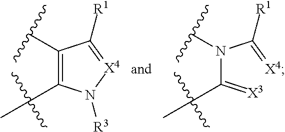
Ring B is selected from phenyl and a 5- to 6-membered heteroaryl ring having 1-3 heteroatoms independently selected from nitrogen, oxygen, and sulfur;
X is selected from N and C;
each of X¹, X³ and X⁴ is independently selected from N and C—R^{x};
R¹ is hydrogen or an optionally substituted group selected from C₁₋₆ aliphatic, phenyl, and a 5- to 6-membered heteroaryl ring having 1-3 heteroatoms independently selected from nitrogen, oxygen, and sulfur;
R² is selected from hydrogen, halogen, NO₂, N(R)₂, OR, N(R)C(O)R, CO₂R, C(O)N(R)₂, and optionally substituted C₁₋₆ aliphatic;
R³ is selected from hydrogen and an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, and a 5- to 6-membered heteroaryl ring having 1-3 heteroatoms independently selected from nitrogen, oxygen, and sulfur;
each R^{x} is independently selected from hydrogen, halogen, or optionally substituted C₁₋₆ aliphatic;
each R is independently selected from hydrogen and an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, and a 5- to 6-membered heteroaryl ring having 1-3 heteroatoms independently selected from nitrogen, oxygen, and sulfur; and
n is 0-3.

2. The compound according to claim 1, wherein the compound is of formula (I-a): 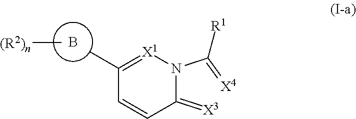
or a pharmaceutically acceptable salt thereof.

3. The compound according to claim 1, wherein the compound is of formula (I-b): 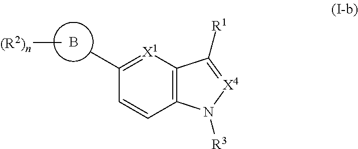
or a pharmaceutically acceptable salt thereof.

4. The compound according to claim 2, wherein the compound is of formula (I-a-i) or formula (I-a-ii): 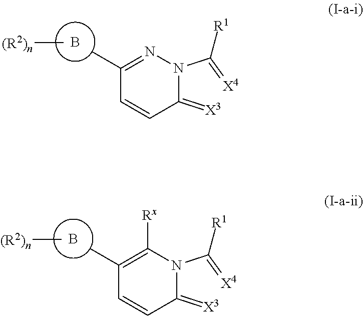
or a pharmaceutically acceptable salt thereof.

5. The compound according to claim 3, wherein the compound is of formula (I-b-i) or formula (I-b-ii): 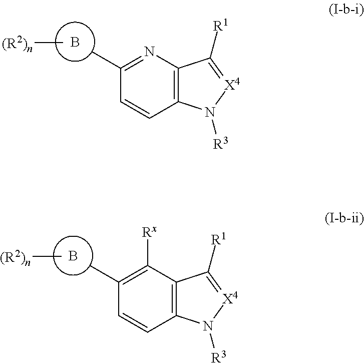
or a pharmaceutically acceptable salt thereof.

6. The compound according to any one of claims 1-5, wherein the compound is not

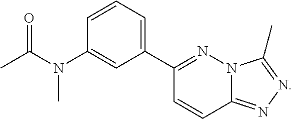 [SMILES: CC(=O)N(C)c1cccc(-c2ccc3nnc(C)n3n2)c1]

7. The compound according to any one of claims 1-6, wherein Ring B is a 5- to 6-membered heteroaryl ring having 1-3 heteroatoms independently selected from nitrogen, oxygen, and sulfur.

8. The compound according to any one of claims 1-6, wherein Ring B is pyridyl.

9. The compound according to any one of claims 1-6, wherein the compound is selected from a compound of formulae (I-a-iii), (I-a-iv), (I-a-v), (I-b-iii), (I-b-iv), and (I-b-v):

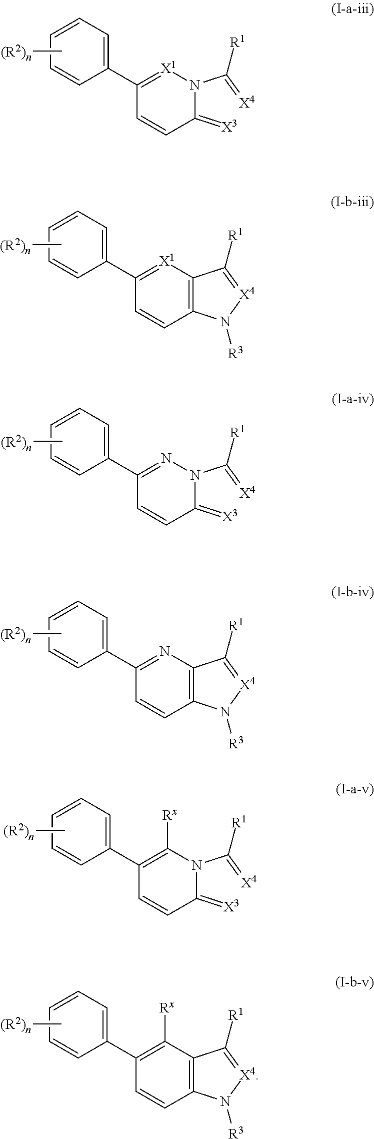

10. The compound according to any one of claims 1, 2, 4, and 7-9, wherein X³ is N.

11. The compound according to any one of claims 1-10, wherein R¹ is hydrogen.

12. The compound according to any one of claims 1-10, wherein R¹ is an optionally substituted group selected from C₁₋₆ aliphatic, phenyl, and a 5- to 6-membered heteroaryl ring having 1-3 heteroatoms independently selected from nitrogen, oxygen, and sulfur.

13. The compound according to claim 12, wherein R¹ is C₁₋₆ aliphatic.

14. The compound according to claim 13, wherein R¹ is methyl.

15. The compound according to claim 14, wherein R¹ is propyl.

16. The compound according to claim 12, wherein R¹ is phenyl.

17. The compound according to claim 12, wherein R¹ is a 5- to 6-membered heteroaryl ring having 1-3 heteroatoms independently selected from oxygen, nitrogen, and sulfur.

18. The compound according to claim 17, wherein R¹ is a 5-membered heteroaryl ring having 1-3 heteroatoms independently selected from nitrogen, oxygen, and sulfur.

19. The compound according to claim 17, wherein R¹ is a 6-membered heteroaryl ring having 1-3 nitrogen atoms.

20. The compound according to claim 19, wherein R¹ is a 6-membered heteroaryl ring having 1-2 nitrogen atoms.

21. The compound according to claim 20, wherein R¹ is selected from

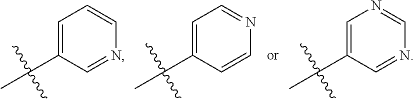 [SMILES: *c1cccnc1 ; *c1ccncc1 ; *c1cncnc1]

22. The compound according to any one of claims 1-21, wherein R^{x} is hydrogen.

23. The compound according to any one of claims 1-21, wherein R^{x} is halogen or optionally substituted C₁₋₆ aliphatic.

24. The compound according to claim 22, wherein R^{x} is optionally substituted C₁₋₆ aliphatic.

25. The compound according to claim 23, wherein R^{x} is C₁₋₆ aliphatic.

26. The compound according to claim 25, wherein R^{x} is methyl.

27. The compound according to any one of claims 1-26, wherein R² is selected from halogen, NO₂, N(R)₂, OR, N(R)C(O)R, CO₂R, C(O)N(R)₂, and optionally substituted C₁₋₆ aliphatic.

28. The compound according to claim 27, wherein R² is halogen.

29. The compound according to claim 28, wherein R² is fluoro.

30. The compound according to claim 27, wherein R² is NO₂.

31. The compound according to claim 27, wherein R² is OR.

32. The compound according to claim 31, wherein R² is OCH₃.

33. The compound according to claim 27, wherein R² is N(R)₂.

34. The compound according to claim 33, wherein R² is NH₂.

35. The compound according to claim 27, wherein R² is N(R)C(O)R.

36. The compound according to claim 35, wherein R² is selected from NHC(O)CH₃ and N(CH₃)C(O)CH₃.

37. The compound according to claim 27, wherein R² is CO₂R.

38. The compound according to claim 37, wherein R² is CO₂H.

39. The compound according to claim 27, wherein R² is C(O)N(R)₂.

40. The compound according to claim 39, wherein R² is C(O)NHCH₃.

41. The compound according to claim 27, wherein R² is optionally substituted C₁₋₆ aliphatic.

42. The compound according to claim 41, wherein R² is CF₃.

43. The compound according to any one of claims 1-42, wherein R is hydrogen.

44. The compound according to any one of claims 1-42, wherein R is an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, a 5- to 6-membered heteroaryl ring having 1-3 heteroatoms independently selected from nitrogen, oxygen, and sulfur.

45. The compound according to claim 42, wherein R is optionally substituted C₁₋₆ aliphatic.

46. The compound according to claim 45, wherein R is C₁₋₆ aliphatic.

47. The compound according to claim 46, wherein R is methyl.

48. The compound according to any one of claims 1-47, wherein R³ is hydrogen.

49. The compound according to any one of claims 1-47, wherein R³ is an optionally substituted group selected from C₁₋₆ aliphatic, a 3- to 7-membered monocyclic carbocyclic ring, a 3- to 7-membered monocyclic heterocyclic ring having 1-3 heteroatoms independently selected from nitrogen, oxygen, and sulfur, phenyl, a 5- to 6-membered heteroaryl ring having 1-3 heteroatoms independently selected from nitrogen, oxygen, and sulfur.

50. The compound according to claim 49, wherein R³ is optionally substituted C₁₋₆ aliphatic.

51. The compound according to claim 49, wherein R³ is C₁₋₆ aliphatic.

52. The compound according to claim 51, wherein R³ is methyl.

53. The compound according to any one of claims 1-52, wherein n is 0.

54. The compound according to any one of claims 1-52, wherein n is 1.

55. The compound according to any one of claims 1-52, wherein n is 2.

56. The compound according to any one of claims 1-55, wherein the compound is: 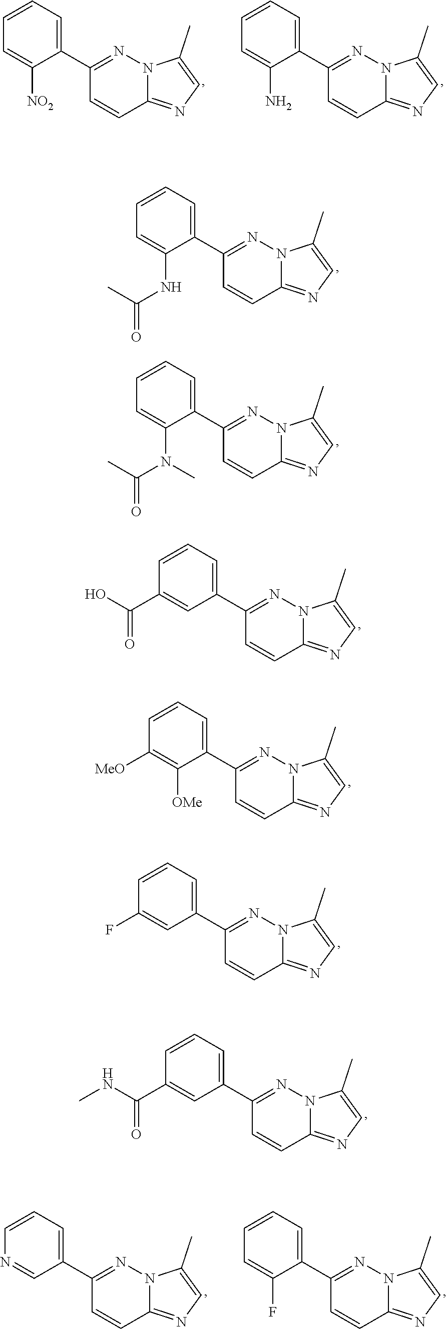 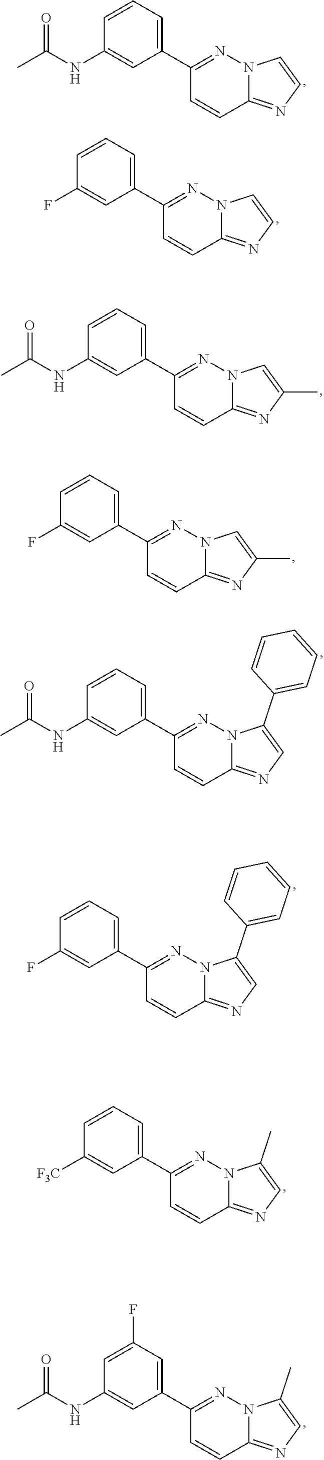 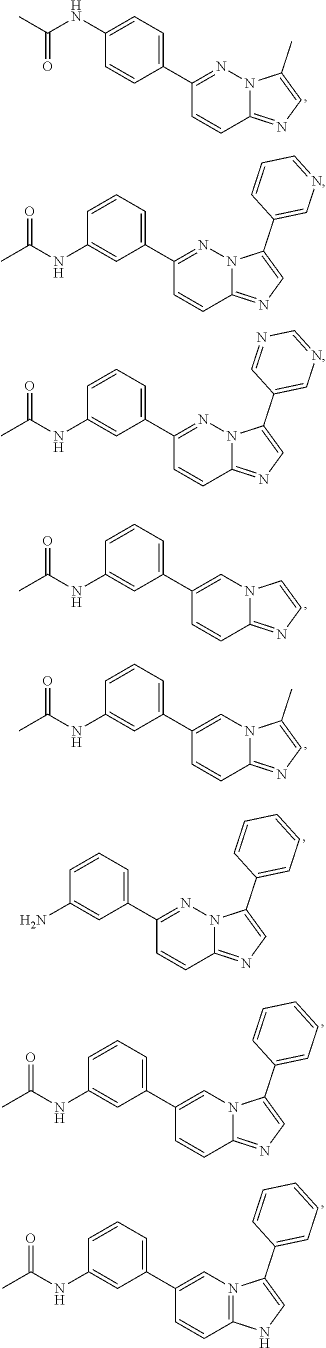 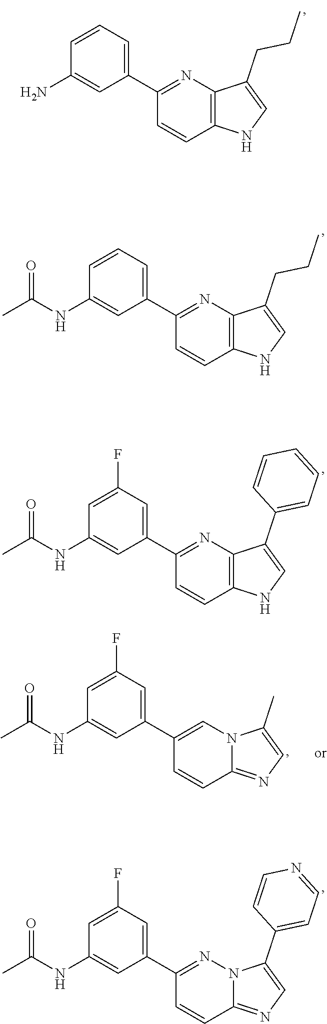
or a pharmaceutically acceptable salt thereof.

57. The compound according to claim 1 or a pharmaceutically acceptable salt thereof, wherein the compound is of formula (II): 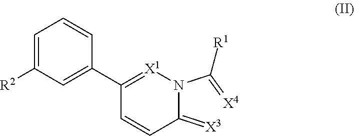
wherein:
R¹ is C₁₋₆ alkyl or C₃₋₆ cycloalkyl;
R² is H, amino, nitro, or acylamino; and
X¹, X³, and X⁴ are each independently N or CH.

58. The compound according to claim 57, wherein at least one of X¹, X³, and X⁴ is N.

59. The compound according to claim 57 or 58, wherein at least two of X¹, X³, and X⁴ are N.

60. The compound according to any one of claims 57-59, wherein each of X¹, X³, and X⁴ is N.

61. The compound of any one of claims 57-60, wherein the compound is not

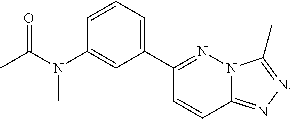 [SMILES: CC(=O)N(C)c1cccc(-c2ccc3nnc(C)n3n2)c1]

62. The compound according to any one of claims 57-61, wherein R¹ is unsubstituted C₁₋₆ alkyl.

63. The compound according to any one of claims 57-61, wherein R¹ is methyl optionally substituted with halogen.

64. The compound according to any one of claims 57-61, wherein R¹ is unsubstituted methyl.

65. The compound according to any one of claims 57-61, wherein R¹ is C₂₋₆ alkyl or C₃₋₆ cycloalkyl.

66. The compound according to any one of claims 57-65, wherein the compound is: 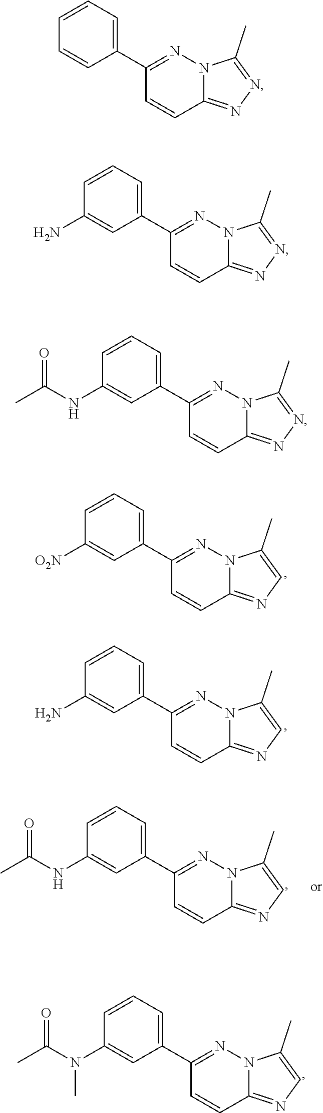
or a pharmaceutically acceptable salt thereof.

67. The compound according to claim 66, wherein the compound is JGJ002, JGJ003, JGJ004, JGJ005, JGJ007, or JGJ008, or a pharmaceutically acceptable salt thereof.

68. The compound according to any one of claims 57-67, wherein:
R² is H, amino, nitro, or —N(R⁵)C(O)R⁶,
R⁵ is H or C₁₋₅ alkyl, and
R⁶ is C₁₋₆ alkyl.

69. The compound according to claim 68, wherein each occurrence of R⁵ is H or CH₃.

70. The compound according to claim 68, wherein R² is —N(R⁴)C(O)R⁵; R⁵ is H; and R⁶ is C₁₋₆ alkyl.

71. The compound according to any one of claims 68-70, wherein X¹ and X³ are each N; and X⁴ is CH.

72. The compound according to claim 68, wherein R² is H, amino, or nitro.

73. The compound according to any one of claims 57-68, wherein R² is NO₂ or —N(R⁵)C(O)R⁶.

74. The compound according to any one of claims 57-73, wherein the compound is 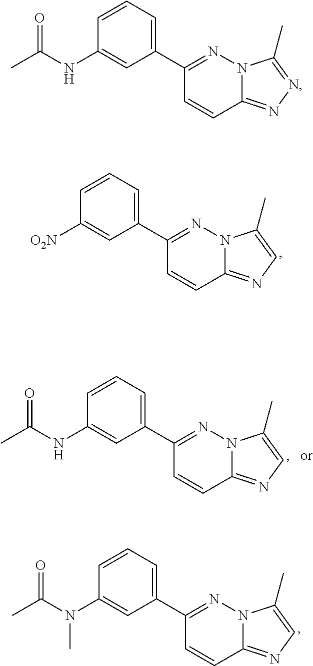 [SMILES: CC(=O)Nc1cccc(-c2ccc3nnc(C)n3n2)c1 ; Cc1cnc2ccc(-c3cccc([N+](=O)[O-])c3)nn12 ; CC(=O)Nc1cccc(-c2ccc3ncc(C)n3n2)c1 ; CC(=O)N(C)c1cccc(-c2ccc3ncc(C)n3n2)c1]
or a pharmaceutically acceptable salt thereof.

75. The compound according to any one of claims 57-74, wherein X¹ and X³ are each N, and X⁴ is CH.

76. The compound according to claim 75, wherein R² is —N(R⁵)C(O)R⁶.

77. The compound according to claim 75, wherein the compound is: 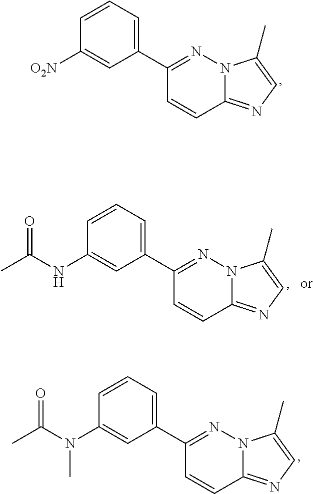 [SMILES: Cc1cnc2ccc(-c3cccc([N+](=O)[O-])c3)nn12 ; CC(=O)Nc1cccc(-c2ccc3ncc(C)n3n2)c1 ; CC(=O)N(C)c1cccc(-c2ccc3ncc(C)n3n2)c1]
or a pharmaceutically acceptable salt thereof.

78. The compound according to claim 77, wherein the compound is JGJ007 or JGJ088, or a pharmaceutically acceptable salt thereof.

79. A pharmaceutical composition comprising a compound of any one of claims 1-56 and a pharmaceutically acceptable excipient.

80. A pharmaceutical composition comprising a compound of any one of claims 57-78 and a pharmaceutically acceptable excipient.

81. A method of inhibiting Lin28 in cells, comprising contacting a cell comprising Lin28 with a compound or composition of any one of claims 57-78.

82. The method of claim 81, wherein the cells are cancer cells, e.g., acute myelogenous leukemia (AML) cells.

83. A method of treating cancer, comprising administering to a subject in need thereof a compound or composition of any one of claims 57-78.

84. The method of claim 83, wherein the subject has a cancer, e.g., acute myelogenous leukemia.

85. A method of treating a cancer, comprising administering to a subject suffering from a cancer or displaying a symptom of a cancer, a compound according to any one of claims 1-78, or a pharmaceutical composition according to claim 79 or 80.

86. The method according to claim 85, wherein the treating is or comprises ameliorating one or more symptoms of the cancer.

87. The method according to claim 85 or 86, wherein the cancer is a hematological cancer.

88. The method according to claim 87, wherein the hematological cancer is acute myelogenous leukemia.

89. The method according to any one of claims 85-88, wherein the compound or pharmaceutical composition is administered in an amount or according to a dosing regimen that has been determined to achieve inhibition of and/or reduced proliferation of a cancer cell.

90. The method according to claim 89, wherein the cancer cell comprises a cancer stem cell.

91. The method according to claim 90, wherein the cancer stem cell comprises a leukemic stem cell (LSC).

92. A method of modulating splicing, the method comprising contacting a splicing-competent system with a compound according to any one of claims 1-78.

93. A method comprising:
contacting a splicing-competent system with a compound of any one of claims 1-78; and
assessing in the system:
(i) presence or level of a splicing product (e.g., a spliced transcript);
(ii) expression or localization of an RNA; and/or
(iii) expression or folding of a polypeptide.

94. A method of modulating splicing in a splicing-competent system by contacting the system with a compound of any one of claims 1-78, so that one or more of the following is observed:
(i) reduced splicing of an RNA;
(ii) altered expression or localization of an RNA; and/or
(iii) altered expression or folding of a polypeptide.

95. A method comprising contacting a splicing-competent system with a compound of any one of claims 1-78, wherein the compound is characterized in that when contacted with a cancer cell it reduces proliferation of the cancer cell relative to that observed in its absence.

96. The method according to any one of claims 92-95, wherein splicing is reduced when the compound is present as compared with when it is absent.

97. The method according to any one of claims 92-96, further comprising assessing splicing in the system as compared with a reference condition.

98. The method according to claim 97, wherein the reference condition is absence of the compound.

99. The method according to claim 97, wherein the reference condition is presence of a control compound.

100. The method according to claim 97, wherein the reference condition is a historical condition.

101. The method according to any one of claims 92-100, wherein the compound inhibits one or more attributes of a splicing machinery component and/or wherein the compound inhibits interaction between or among splicing machinery components.

102. The method according to any one of claims 92-101, wherein the compound binds directly to one or more splicing machinery components, or complexes thereof.

103. The method according to claim 101 or 102, wherein the splicing machinery component is an RNA component.

104. The method according to claim 101 or 102, wherein the splicing machinery component is a polypeptide component.

105. The method according to claim 101 or 102, wherein the splicing machinery component is selected from the group consisting of RNA components, polypeptide components, and complexes thereof or therebetween.

106. The method according to claim 103 or 105, wherein the RNA component is or comprises a small nuclear RNA (snRNA).

107. The method according to claim 106, wherein the snRNA is selected from the group consisting of: U1, U2, U4, U5, and U6.

108. The method according to claim 104 or 105, wherein the polypeptide component is or comprises an Sm polypeptide or an Lsm polypeptide.

109. The method according to any one of claims 104, 105, or 108, wherein the polypeptide component is selected from the group consisting of: Prp3, Prp31, Prp4, CypH, 15.5K, Prp8, Brr2, Snu114, Prp6, Prp28, 40K, Dib1, Snu66, Sad1, and 27K.

110. The method according to claim 109, wherein the splicing machinery component comprises a Prp31 polypeptide.

111. The method according to claim 105, wherein the splicing machinery component comprises a U4 snRNA, a U6 snRNA and a Prp31 polypeptide.

112. The method according to any one of claims 92-111, wherein the compound inhibits an interaction between: a U6 snRNA and a Prp31 polypeptide; or a U4 snRNA and a Prp31 polypeptide.

113. The method according to any one of claims 92-112, wherein the compound inhibits an activity of a Prp31 polypeptide.

114. The method according to any one of claims 92-113, wherein the contacting occurs in vitro, ex vivo or in vivo.

115. The method according to any one of claims 92-114, wherein the splicing-competent system is a cancer cell.

116. The method according to claim 115, wherein the cancer cell comprises a cancer stem cell.

117. The method according to claim 116, wherein the cancer stem cell comprises a leukemic stem cell (LSC).
